(19) United States
(12) Patent Application Publication
(10) Pub. No.: US 2013/0171172 A1
(43) Pub. Date: Jul. 4, 2013

(54) CALPAIN-3 INHIBITORS FOR TREATING MUSCULAR DYSTROPHIES AND CARDIOMYOPATHIES

(71) Applicants: Isabelle Richard, Corbeil-Essonnes (FR); Karine Charton, Villabe (FR)

(72) Inventors: Isabelle Richard, Corbeil-Essonnes (FR); Karine Charton, Villabe (FR)

(73) Assignees: Genethon, Evry (FR); Universite D'Evry Val D'Essonne, Evry (FR)

(21) Appl. No.: 13/807,089

(22) PCT Filed: Jun. 30, 2011
(86) PCT No.: PCT/EP2011/061042

§ 371 Date: Feb. 13, 2013

(30) Foreign Application Priority Data
Jul. 1, 2010 (EP) 1055333

(51) Int. Cl. A61K 39/395; A61K 38/02; A61K 31/7105

(52) U.S. Cl. CPC A61K 39/3955; A61K 31/7105; A61K 38/02
USPC 424/172.1; 514/44 A; 514/1.1; 514/44 R; 435/6.12; 436/501; 435/7.92

(57) ABSTRACT

A composition comprising a calpain-3 inhibitor for treating muscular dystrophies and cardiomyopathies, in particular tibial muscular dystrophy (TMD).

### BACKGROUND

The present disclosure relates to the treatment of diseases affecting the striated muscle (skeletal muscles and/or heart muscle), and in particular of tibial muscular dystrophy (TMD).

It advocates the identification and the use of calpain-3 inhibitors as a medicine for treating these diseases.

### DISCUSSION OF PRIOR ART

Neuromuscular diseases gather various pathologies which are generally associated with a temporary or permanent loss of muscular strength. Such a loss of strength most often comes along with a wasting of muscle tissue, also called amyotrophy.

Among such muscular diseases, myopathies form an important group corresponding to damages to the actual muscle fiber. Among these, progressive muscular dystrophies are characterized by a decrease of the muscular strength, generally along with a muscular atrophy as well as by anomalies visible in muscular biopsy, which reveal a modification of the tissue. This group especially comprises Duchenne muscular dystrophy (or DMD), Becker muscular dystrophy (or DMB), proximal limb-girdle muscular dystrophies or distal muscular dystrophies such as tibial muscular dystrophy (or TMD).

Tibial muscular dystrophy (TMD) has been highlighted for the first time in consanguine Finnish families (Udd et al., 1993).

Tibial muscular dystrophy is characterized by a muscular atrophy and weakness generally confined to the front compartment of the lower limbs, mainly the Tibialis Anterior (TA). The patients' calf has a prominence in its ventral part. The first symptoms often appear asymmetrically and tend to become bilateral along time (Udd et al., 1998). The first clinical symptoms appear when the patients are around 35 years of age (Udd et al., 1993). The disease develops slowly and most patients can still walk, without however being able to walk on their heels, and in the most severe cases, they need a walking aid.

No heart impairment or face impairment has been observed. The CPK rate remains normal or moderately high (Udd et al., 1998). However, a strong heterogeneity has been pointed out on a panel of TMD patients (forming close to 9% of the total TMDs), with variable disorders capable of affecting other muscles such as the Quadriceps and the Soleus, but also sometimes distal and proximal impairments that may even imply the arm muscles (Udd et al., 2005). Magnetic resonance imaging (MRI) carried out on different patients shows that the muscle is replaced with connective and adipose tissue in the TA and, according to the affection, in the Quadriceps and the Soleus.

The patients' muscular biopsies reveal myopathic-dystrophic changes of variable severity comprising a modification of the size of muscular fibers (often, the presence of larger fibers), internalized nuclei, and a few basophile-positive or necrosed fibers (Udd et al., 1992); (Partanen et al., 1994). Further, apoptotic nuclei have been found on biopsies of TMD patients' Tibialis (Haravuori et al., 2001). Biopsies visualized in electron microscopy show, variably according to patients, the presence of a few vacuoles containing cellular debris while the sarcomere structure is preserved (Udd et al., 1993).

In 2002, Isabelle Richard and Bjarne Udd's teams have highlighted the most frequent mutation in Finland, in the gene encoding the largest protein of living beings: titin (Hackman et al., 2002).

Titin (OMIM #18840, also called connectin) is a giant protein encoded by a gene having an approximate 294-kb size located on chromosome 2, region 2q31, and formed of 363 exons coding for 381,388 amino acids. The molecular mass of titin ranges from 3,000 to 3,700 kDa (Bang et al., 2001).

Titin extends from the Z disc to the M line of the sarcomere (Furst et al., 1988; Wang et al., 1979) which corresponds to a half-sarcomere. An essential protein for the organization and the integrity of sarcomeres, it forms the third largest network of filaments in the striated muscle, amounting for some 10% of the myofibrillar mass after those of actin and of myosin, and is the only continuous system along the entire sarcomere length. The N-terminals parts of two titin molecules belonging to two adjacent sarcomeres are anchored in the Z band where they overlap in antiparallel (Gregorio et al., 1998). The C-terminal region takes part in the structure of the M-line of the sarcomere, where two titin molecules of adjacent sarcomeres superpose (Obermann et al., 1997); (Furst et al., 1999). Last, the central titin portion (the I band) forms the elastic part of the molecule. Due to its size and to its many protein interactions, titin plays an important part in the assembly of sarcomeres, the maintaining of contractile elements during the contraction, the integration of thick filaments in the sarcomere, controls the integrity, and provides the mechanical stability of the sarcomere. Further, during muscular work, the deployment of titin generates a force capable opposing to the sarcomere stretching tension: during the contraction of a striated muscle, the active tension results from the action of the thin actin filaments on the thick myosin filaments and the passive tension results from the extension of titin. Last, it plays an important part in signaling pathways (Trinick, 1994) (Labeit and Kolmerer, 1995) (Gregorio et al., 1999) (Machado and Andrew; 2000) (Squire, 1997) (Clark et al., 2002) (Lange et al., 2005), especially since it has a great number of partners all along the sarcomere.

A mutation detected on Finnish patient, FINmaj (major in Finland), has been identified in the last exon of titin (Hackman et al., 2002). This mutation in the heterozygous state causes TMD. Mutation Final is the consequence of an 11 base pair deletion/insertion (AAGTAACATGG->TGAAAGAAAAA) causing the modification of 4 acid amino acids into 4 basic amino acids E33359_W33362delinsVKEK (EVTW->VKEK) in a highly conserved hydrophobic area of the m10 domain of titin (Hackman et al., 2002). Such a mutation seems to be the result of recombination of the DNA with a same sequence present in an intron located in the A band of titin between exons 246 (Fn3) and 247 (Ig) of the healthy gene, 87,204 base pairs upstream of the last exon, Mex6. In this mutation, tryptophan is muted into charged lysine. Now, such a tryptophan residue is generally highly conserved in all Ig repeats of titin and is known to be of crucial importance in the stabilizing of the hydrophobic nucleus to the three-dimensional structure of the Immunoglobulin-like domain (Improta et al., 1998).

Mutation FINmaj has been highlighted in a large number of Finnish patients and its prevalence would be greater than 10 cases per 100,000 in Finland (Hackman et al., 2002). Six other missense or nonsense mutations have been highlighted in the last two exons of titin in Belgian, French, Italian, and Spanish patients (Hackman et al., 2008; Hackman et al., 2002; Pollazzon et al., 2009; Van den Bergh et al., 2003), resulting in the same phenotypes as mutation FINmaj.

Interestingly, there would be a genotype-phenotype correlation, especially visible after the pointing out of a much more severe impairment in patients having a mutation causing the production of a truncated protein resulting from a missense mutation or from a frameshift mutation causing a premature stop (Hackman et al., 2008).

An epitope retrieval has shown that the C-terminal part of titin is 50% absent (Hackman et al., 2008).

Among the many partners of titin, it has been shown that calpain 3 would bind to the single encoded region by the penultimate exon of titin called Mex5 (encoding for the is7 domain) of titin (Kinbara et al., 1997).

Calpains are a family of non-lysosomal cystein proteases activated by calcium. This family currently comprises 11 members, including 2 ubiquitous proteins (calpain 1 and 2). The physiological functions of calpains still remain widely unknown. As regulating-type proteases, they probably regulate important cell functions.

Calpain 3 (also called p94) belongs to the family of calpains, which all are cystein proteases (Dear et al., 1999). Calpain 3 is coded by a gene formed of 24 exons distributed over a 35-kb region of chromosome 15. The 94 kDa protein comprises 821 amino acids and is comprised of four domains which are also present in the members of the calpain family. The first biochemical characterizations of calpain 3 have shown that this enzyme would autolyze in the form of two main fragments (Sorimachi et al., 1993). Such an autolysis, which takes place in the IS1 domain, forms the protease activation mechanism and is described as being sequential (Taveau et al., 2003). Thus, three autolysis sites (S1, S2, and S3) have been highlighted in IS1, resulting in the forming of a short fragment of 34 kDa and of shorter fragments between 55 and 60 kDa (Kinbara et al., 1998).

When the protein is integral, an a helix coded by sequence IS1 closes the catalytic cleft, thus preventing substrates and inhibitors from penetrating into it (Diaz et al., 2004). When it autolyzes, it releases the IS1 fragment (intramolecular cleavage) which then makes the catalytic cleft accessible. The protein, which has become active, can then cleave its substrates but also other calpain-3 molecules (intermolecular cleavage). In a muscle, calpain 3 is present in majority in non-autolyzed 94-kDa form (Kinbara et al., 1998; Sorimachi et al., 1990), which suggests that it is mainly present in inactive form and that an activation enabling to activate it is necessary.

The exact function of calpain 3 in the skeletal muscle is not known, but its deficiency is responsible for LGMD2A. Recessive mutations in the calpain-3 gene are the cause for LGMD2A (Limb Girdle Muscular Dystrophy 2A) (Richard et al., 1995), which is the most common form of limb-girdle dystrophies (it amounts to from 30 to 40% thereof).

There obviously is a persistent need for the development of new medical products enabling to treat the large spectrum of pathologies affecting the striated muscle, and especially muscular dystrophies.

Thus, and up to now, no specific treatment is known for TMD. Most patients require kinesitherapy to prevent the aggravation of contractures. An obvious approach is the provision of a gene coding the normal protein, but this strategy cannot be applied in the case of titin, due to the large size of the gene, which exceeds the capacities of currently-used transfer vectors.

### SUMMARY

The present invention provides new therapeutic approaches to treat certain forms of muscular dystrophies—and especially TMD—or cardiomyopathies, and provides the possibility of identifying new medicines intended for such pathologies.

Indeed, the present invention is based on the highlighting by the Applicant of the fact that a partial inhibition of calpain 3 enables to improve certain forms of muscular dystrophies, and especially TMD.

Thus, and according to a first aspect, the present invention relates to a composition comprising a calpain-3 inhibitor for treating diseases affecting the striated muscle, in particular muscular dystrophies and cardiomyopathies, and more specifically still those associated with an overexpression or an overactivation of calpain 3.

In other words, the present invention provides using a composition comprising a calpain-3 inhibitor to prepare a medicine for treating muscular dystrophies and cardio-myopathies.

Said composition may further contain any acceptable compound or excipient, especially pharmaceutically acceptable. It may further comprise other active principles intended to treat the same pathology or another pathology. According to a specific embodiment, the calpain-3 inhibitor(s) are the only active principles of the composition.

It may also be envisaged to associate, in the same composition, at least two calpain-3 inhibitors of different nature, for a simultaneous or sequential administration.

The administration may be intramuscular as well as intravenous, or even subcutaneous, intraperitoneal, or oral.

The present invention is thus based on the highlighting of the therapeutic value of calpain-3 inhibitors in the context of the present invention.

“Calpain-3 inhibitor” designates any molecule capable of decreasing or reducing the activity of calpain 3. Since calpain 3 potentially has many in vivo biological implications, advantage is given to a suppression which is never total.

Similarly, a specific calpain-3 inhibitor is advantageously used in the context of the present invention. “Specific” means that it exclusively or mainly inhibits calpain 3 but not other proteins biologically active in vivo. In particular, it appears to be very important to verify the specificity of the inhibitor regarding proteins close to calpain 3, that is, the other calpains, and especially ubiquitous calpains 1 and 2. The advantage of a specific inhibitor is that it generally provides a greater selectivity and efficiency. Clinically, this results in less possible side effects.

The reduction or inhibition of the activity of calpain 3 may result from 2 different modes of action of the inhibiting molecules:

According to a first embodiment, the molecule decreases the expression and/or the quantity of produced calpain 3, by especially acting on the transcription/translation of the calpain-3 gene. This for example is the mode of action of antisense oligonucleotides, of silencing RNAs, of short hairpin RNAs, and of ribozymes, which are preferred inhibitors in the sense of the present invention.

Such molecules can easily be identified by those skilled in the art, especially based on the calpain-3 gene sequence. A particularly relevant sequence for human therapy is that of the human calpain-3 gene, of sequence SEQ ID NO: 1.

Since experiments are previously made on mice, the murine calpain-3 gene sequence is also of interest and is illustrated in sequence SEQ ID NO: 2.

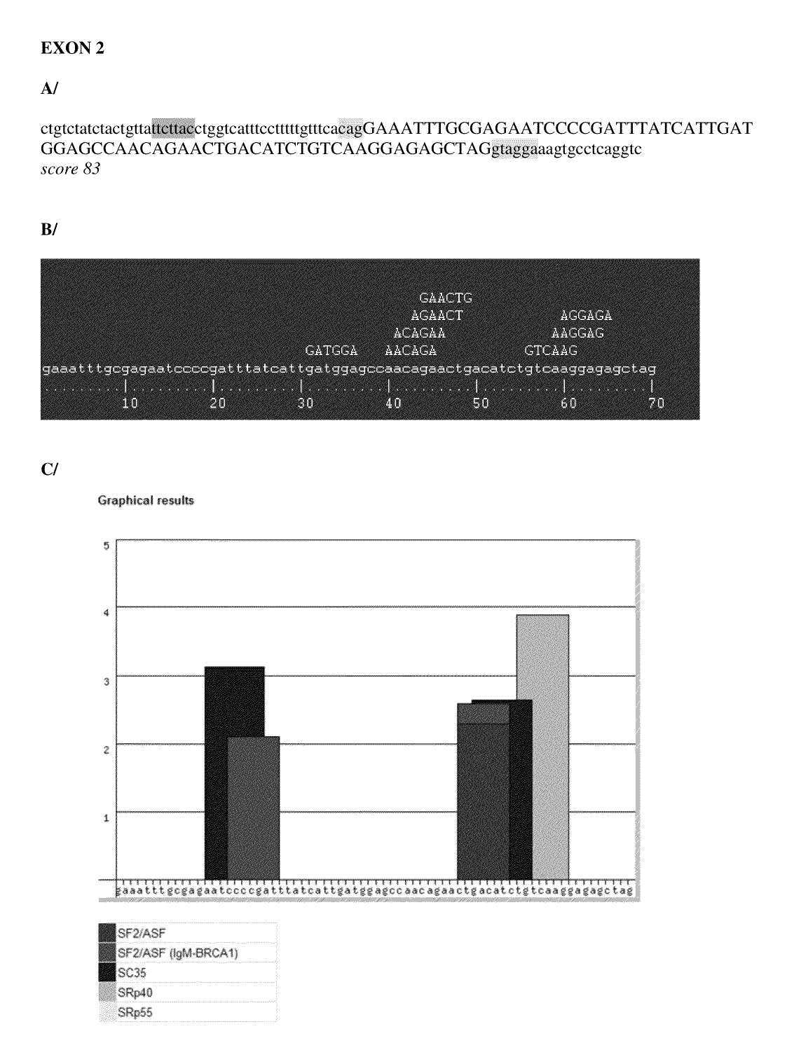

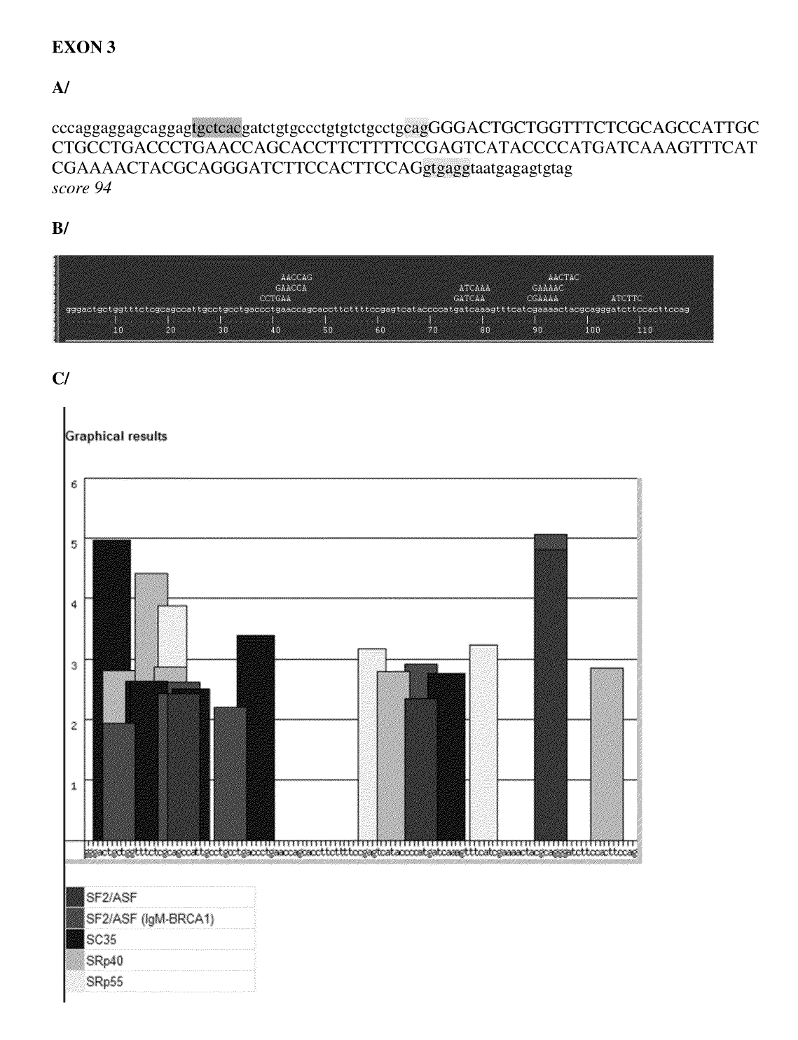

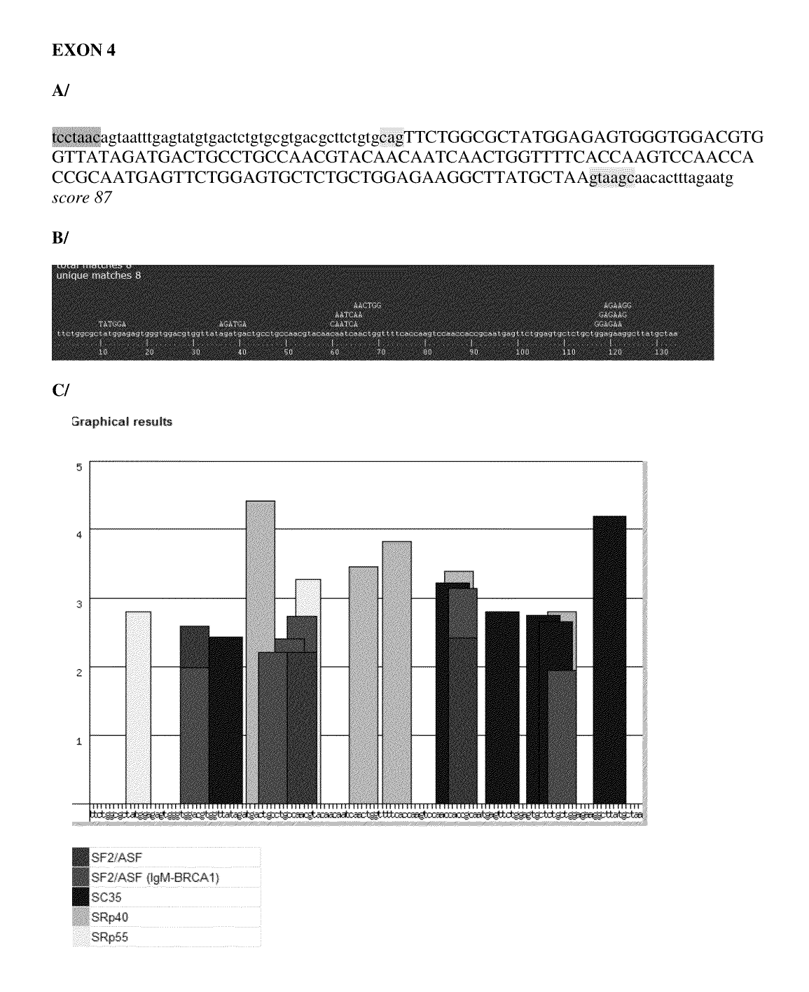

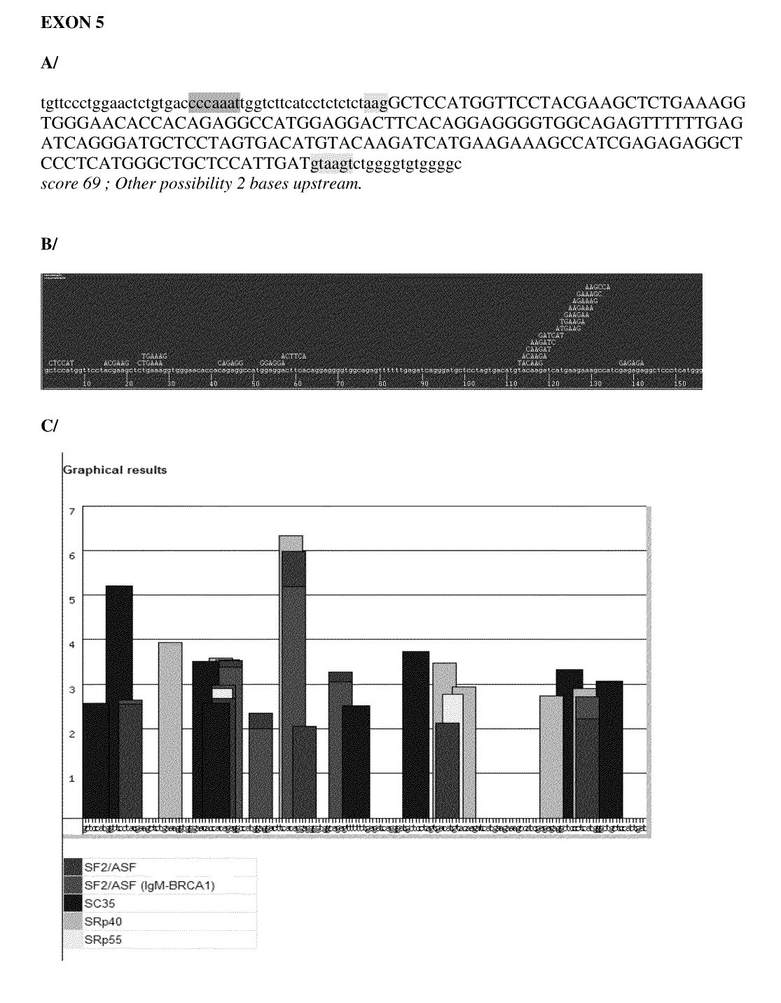

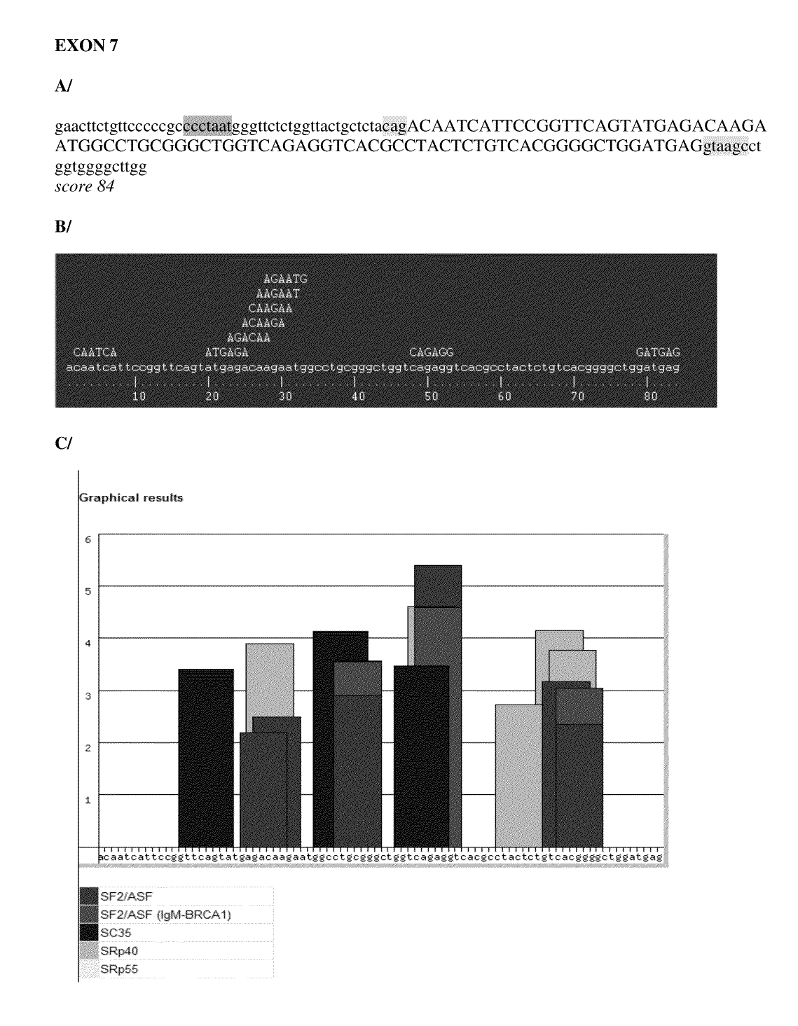

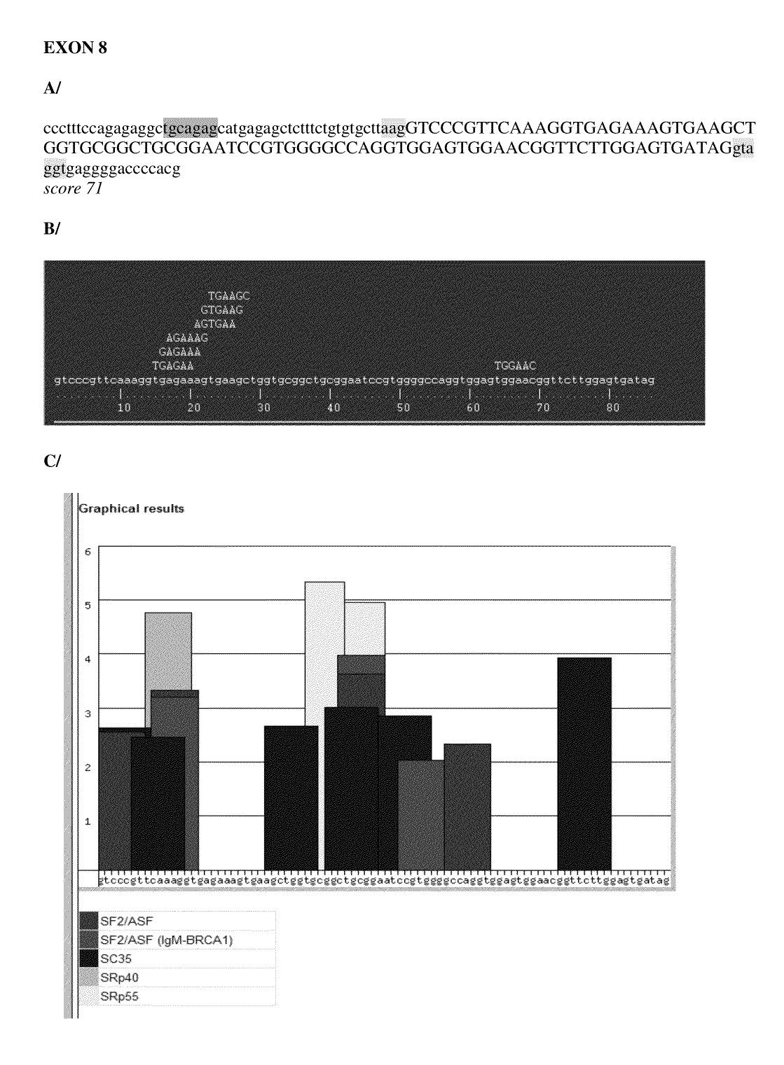

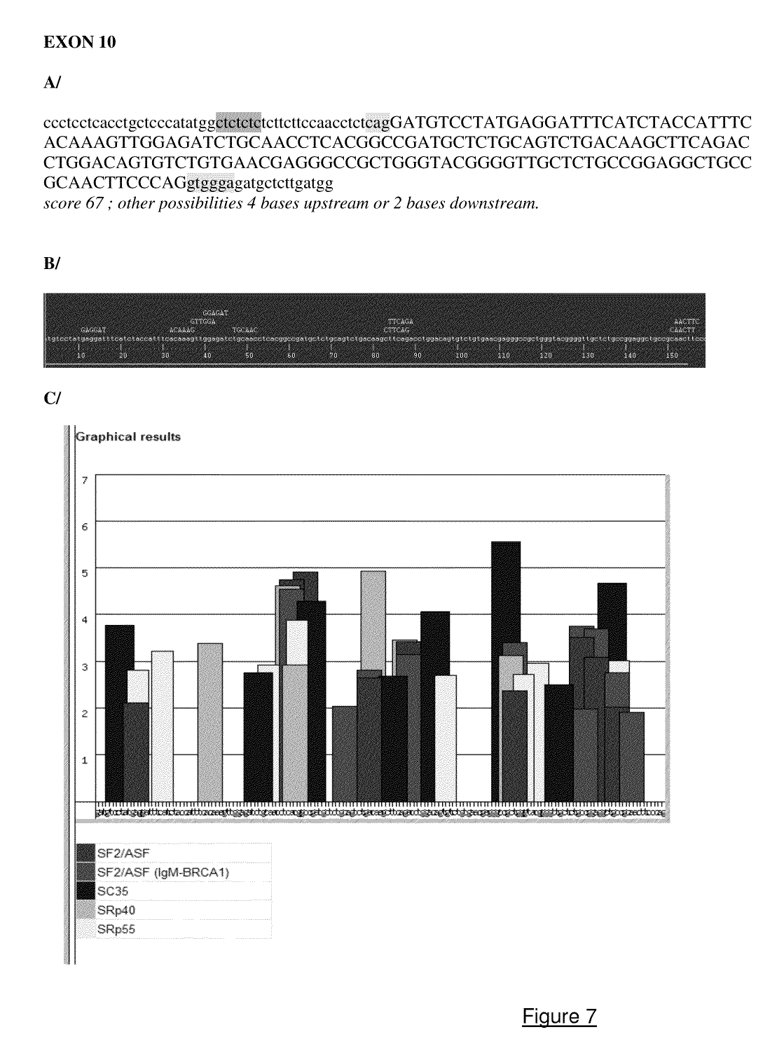

Antisense sequences capable of introducing exon skipping, to eliminate an exon critical for the proteolytic activity or inducing an interruption of the reading frame at the beginning of a sequence are preferred candidates. The targeted exons advantageously are exons 2, 3, 4, 5, 7, 8, and 10. At the level of the corresponding sequences, the important sequences for the splicing (branch point or BP, splicing donor and acceptor sites, splicing factor binding ESE (Exonic Sequence Enhancer) sites (SR proteins)) are preferred. They may easily be determined by those skilled in the art and are illustrated, in relation with the human gene of sequence SEQ ID NO: 1, in FIG. 7.

They may for example be the following antisense sequences:

| Targeted | | |
|---|---|---|
| exon | Name | Sequence |
| ex3 | ex3_BP | acagaucGuGAGCAcuccugc |
| | | (SEQ ID NO: 18) |
| | ex3_ESE1 | GGCuGCGAGAAACCAGCAGuCC |
| | | (SEQ ID N: 19) |
| | ex3_ESE2 | uCCCuGCGuaguuuuCGAuGAA |
| | | (SEQ ID NO: 20) |
| ex4 | ex4_BP | auuacuGuuAGGAaauguguc |
| | | (SEQ ID NO: 21) |
| | ex4_ESE1 | ACGuuGGCAGGCAGuCAuCuAuA |
| | | (SEQ ID NO: 22) |
| | ex4_ESE2 | uCCAGAACuCAuuGCGGuGGuu |
| | | (SEQ ID NO: 23) |
| ex5 | ex5_BP | agaccaAuuuGGGgucacaga |
| | | (SEQ ID NO: 24) |
| | ex5_ESE1 | CuGuGAAGuCCuCCAuGGCCuCu |
| | | (SEQ ID NO: 25) |
| | ex5_ESE2 | uGGCuuuCuuCAuGAuCuuGuA |
| | | (SEQ ID NO: 26) |

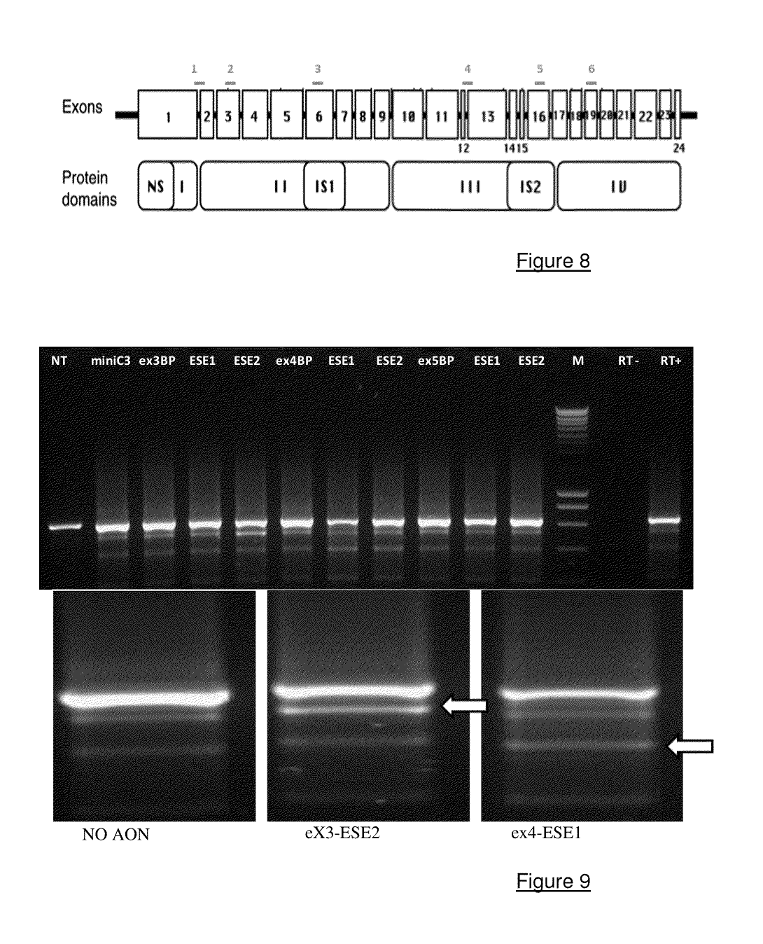

As a variation, an si (“small interference”) type interfering RNA also is a preferred inhibitor according to the present invention. As an example, to illustrate the present invention, the following oligonucleotides, having their position shown in FIG. 8, are capable of performing this function, both in mice and in men:

| | |
|---|---|
| TGGAAGAAGACCTCCGGAAA | (SEQ ID NO: 3) |
| CCCATGATCAAAGTTTCAT | (SEQ ID NO: 4) |
| AACCTCTCCTTCTGGTCTGAACA | (SEQ ID NO: 5) |
| CCAAAGAGATGCACGGGAA | (SEQ ID NO: 6) |
| GCAACAAGGAGCTGGGTGT | (SEQ ID NO: 7) |
| ACAAGGACCTGAAGACACA. | (SEQ ID NO: 8) |

It is within the abilities of those skilled in the art to verify whether the molecule tested as a calpain-3 inhibitor affects the state of expression and/or of production of calpain 3, especially by means of the following well-known techniques:
transcript expression state by Northern blot or PCR;
protein production state by antibody detection (Western blot or ELISA).

According to a second embodiment, calpain 3 is produced “normally” but its activity is affected by the considered inhibitor. In this case, the inhibitor typically is a calpain-3 antibody, a chemical molecule, a protein, or a peptide having the desired inhibiting activity.

The activity of calpain 3 may be measured by means of different tests, especially those described in documents WO 2003/002730, WO2007/039699, and Milic et al. (2007).

Specific calpain-3 antibodies are available and may be used in the context of the present invention. It may for example be the antibody described in Baghdiguian et al. (1999).

The proteins and peptides of interest are advantageously selected from among those having an in vivo inhibiting activity for calpain 3. Thus, Isabelle Richard's team has recently demonstrated that the myospryne protein (CMYA5, cardiomyopathy-associated 5) of sequence SEQ ID NO: 9 (human) or SEQ ID NO: 10 (murine), is capable of performing this function. An active fragment thereof, for example, having SEQ ID NO: 11 (human) or SEQ ID NO: 12 (murine), may also be used in the context of the present invention. The exogenous provision of such active derived proteins or peptides thus results in reducing in vivo the activity of calpain 3 and thus of improving the pathological states targeted by the present invention.

Last, the above-described activity tests allow a conventional pharmacological approach, that is, the identification of chemical molecules having the desired inhibiting activity. To achieve this, banks of available molecules may be used.

More generally, and according to a second aspect, the present invention relates to a method for identifying or screening medical products for the treatment of muscular dystrophies, advantageously of TMD, or of cardiomyopathies.

The inhibiting capacity of the tested compound on calpain 3 thus has to be assessed.

This method is advantageously implemented in vitro.

Here again, the inhibiting power may be assessed either by measuring the expression or the production of calpain 3, or by measuring its activity by means of the previously-mentioned techniques.

Such measurements are carried out in parallel in the presence and in the absence of the tested compound, and then compared.

In the case where a decrease of calpain 3 is observed in the presence of the tested compound, said compound becomes a candidate medicine for the treatment of the targeted pathologies.

The targeted pathologies thus are those associated with an overexpression or an overactivation of calpain 3. Advantageously, the present invention finds applications in the treatment of muscular dystrophies, affecting skeletal muscle. Pathologies of the striated muscles, and thus also those affecting the heart muscle, called cardiomyopathies, are more generally targeted.

Very clearly, for the first time, the present invention highlights the possibility of treating tibial muscular dystrophy (TMD) with this strategy.

More generally, the present invention reveals, for the first time, the correlation between muscular dystrophies and a possible overactivation of calpain 3. It should be reminded that to date, only limb-girdle dystrophies due to a deficient calpain 3 are identified in relation with calpain 3. The present invention highlights the existence of muscular dystrophies possibly due to an overexpression or overactivation of calpain 3, which may be called “dominant calpainopathies” and be treated by means of the provided strategy.

Similarly, the present invention aims at the treatment of cardiomyopathies possibly due to an overexpression or overactivation of calpain 3, or to an exogenous supply of calpain 3. Indeed, calpain 3 is expressed in the heart, although much more weakly than in the skeletal muscle (Taveau et al., 2002) and Isabelle Richard's team has shown that beyond a threshold, calpain 3 could bring about deleterious effects on the heart muscle.

The foregoing features and advantages of the present invention will be discussed in the following description of the following embodiments in connection with the accompanying drawings. They however are not limiting.

The present invention is further illustrated in relation with mouse TMD.

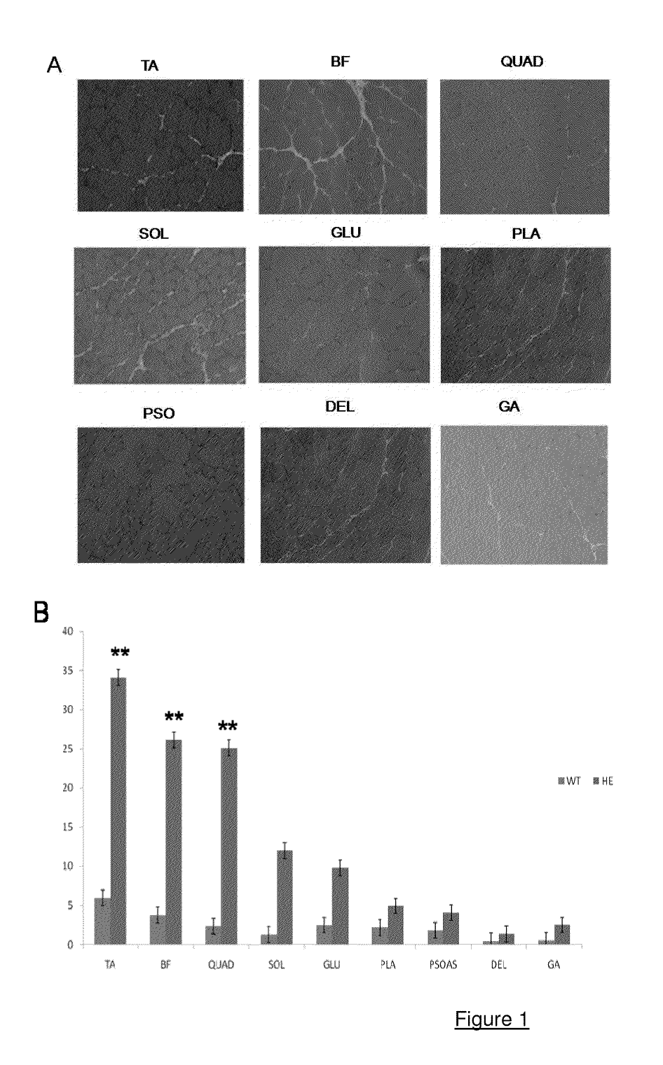

FIG. 1: A/Histology of muscles of mice heterozygous for the FINmaj mutation and WT mice. TA=Tibialis Anterior, QUA=Quadriceps, BF=Biceps Femoris, SOL: Soleus, GLU: Gluteus, PLA: Plantaire, PSO: Psoas, DEL: Deltoid, GA: Gastrocnemius). TA, QUAD, and BF damage in mice heterozygous for the FINmaj mutation at 9 months of age. B/Quantification of the centronucleation of the fibers of muscles TA, QUA, and BF of HE and WT mice.

FIG. 2: Western Blot performed on WT and HE mice muscle homogenates, hybridized with calpain-3 and actin antibodies. Quantification of the ratio of the quantity of calpain 3 in the muscles to the actin used as a normalizer in arbitrary units (UA) (n=4).

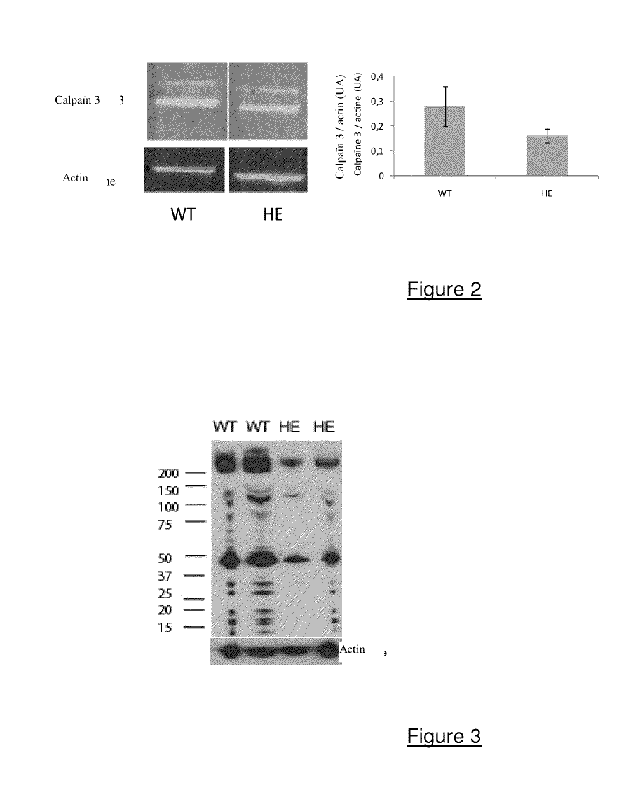

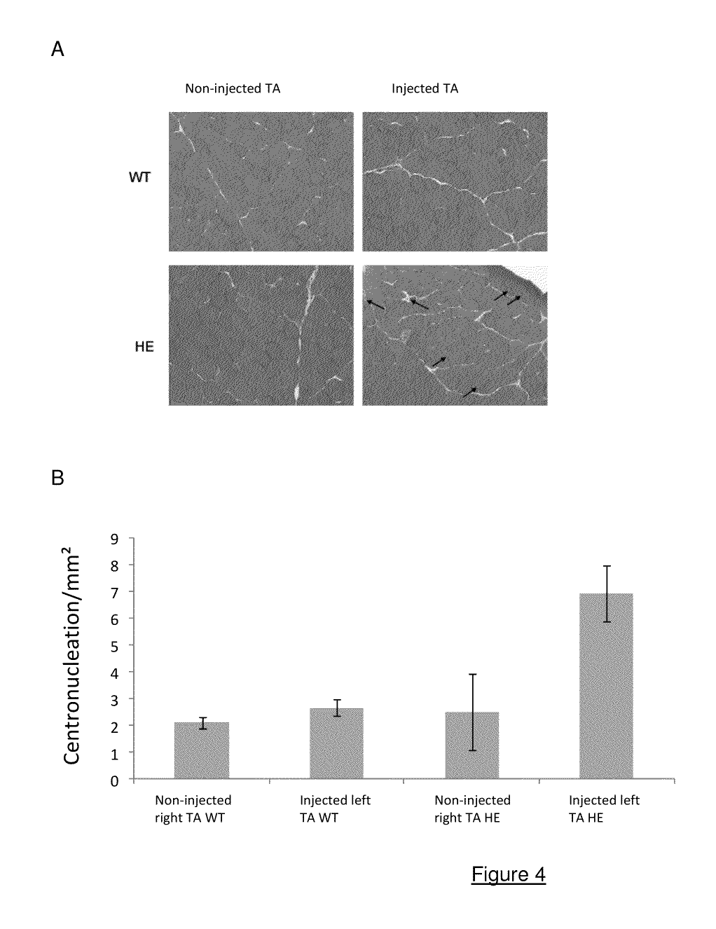

FIG. 3: Marking of titin degradation bands with an antibody directed against the terminal part of titin (coded by the last exon) after subcellular fractionation of the Psoas of the WT and HE mice for the FINmaj mutation.

FIG. 4: A/Histology of muscles heterozygous for the FINmaj mutation and WT after IM injection of the AAV encoding for human calpain 3. B/Quantification of the centronucleation of the TA fibers injected and non-injected with the C3 AAV of WT and HE mice.

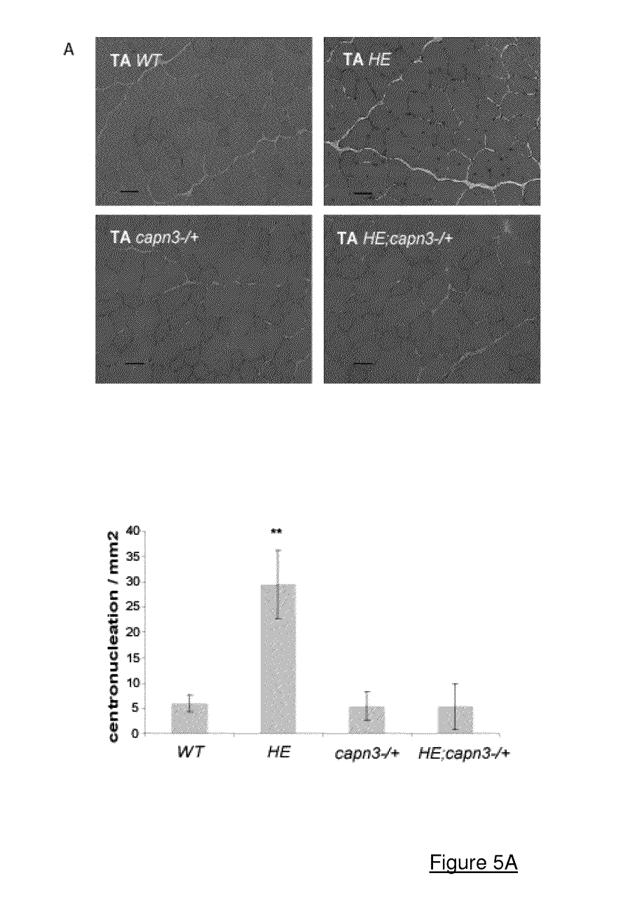

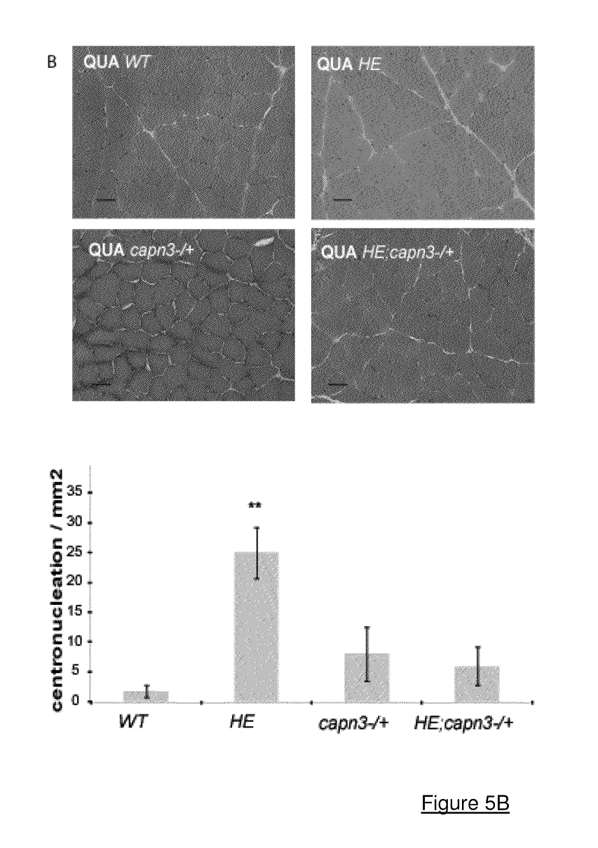

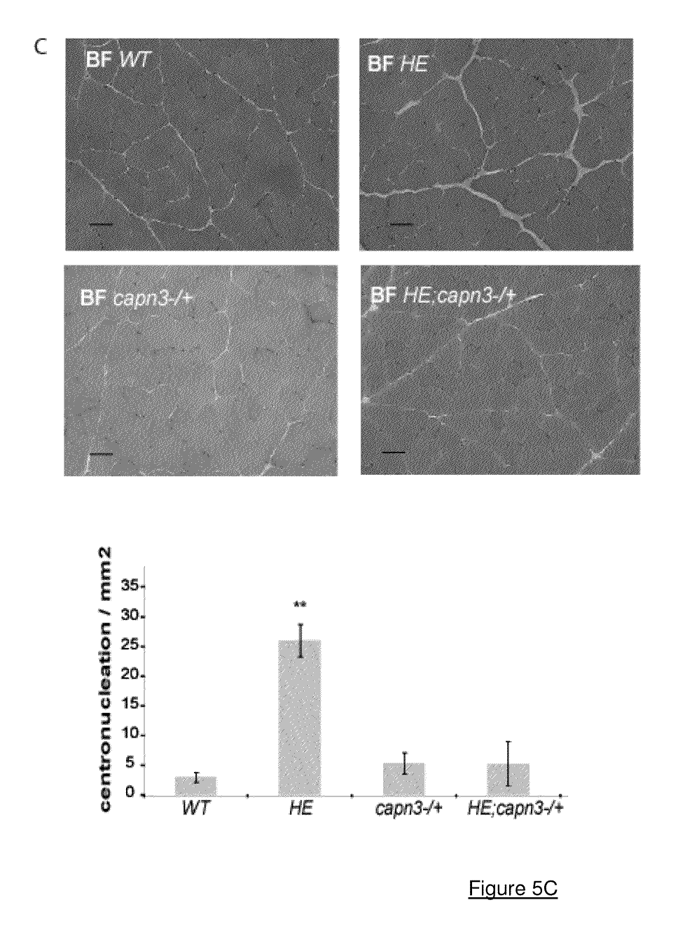

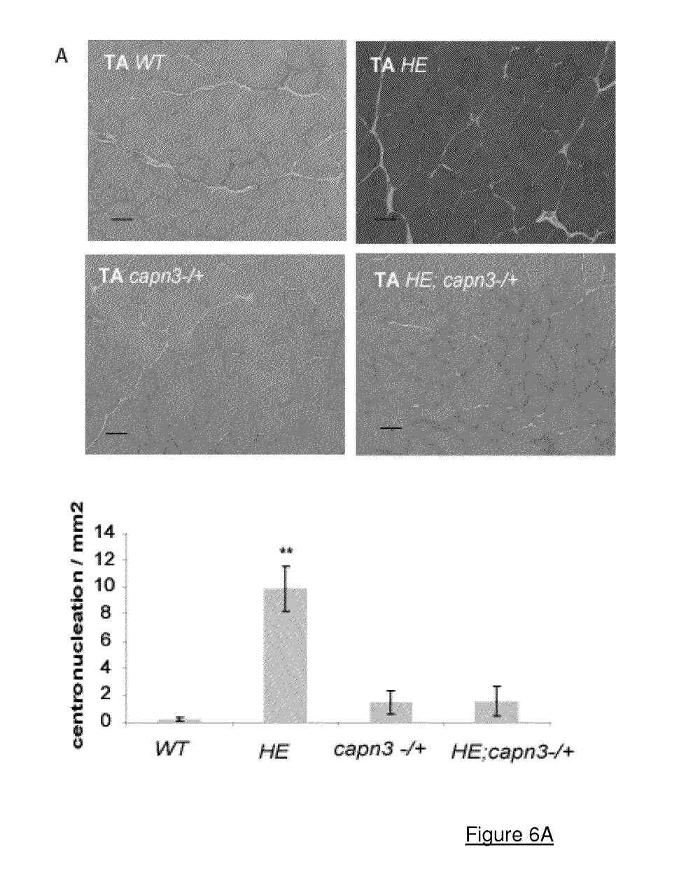

FIG. 5: Histology of the muscles of HE and WT, capn3+/− and HE/capn3+/− mice at 9 months of age and quantification of the centronucleation. A/TA B/QUAD C/BF. Bar=50 μm.

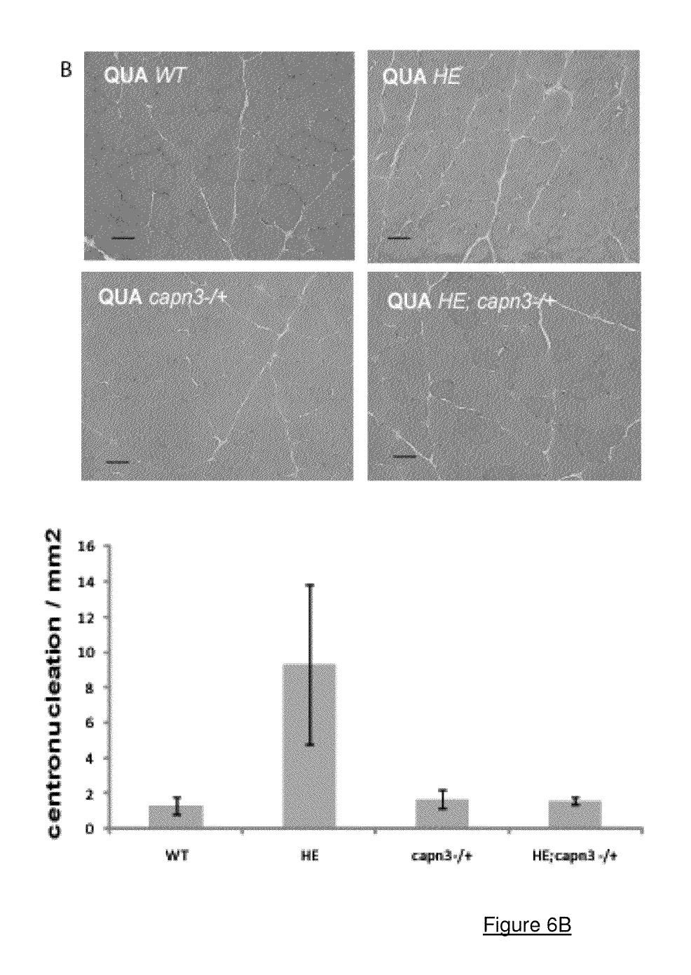

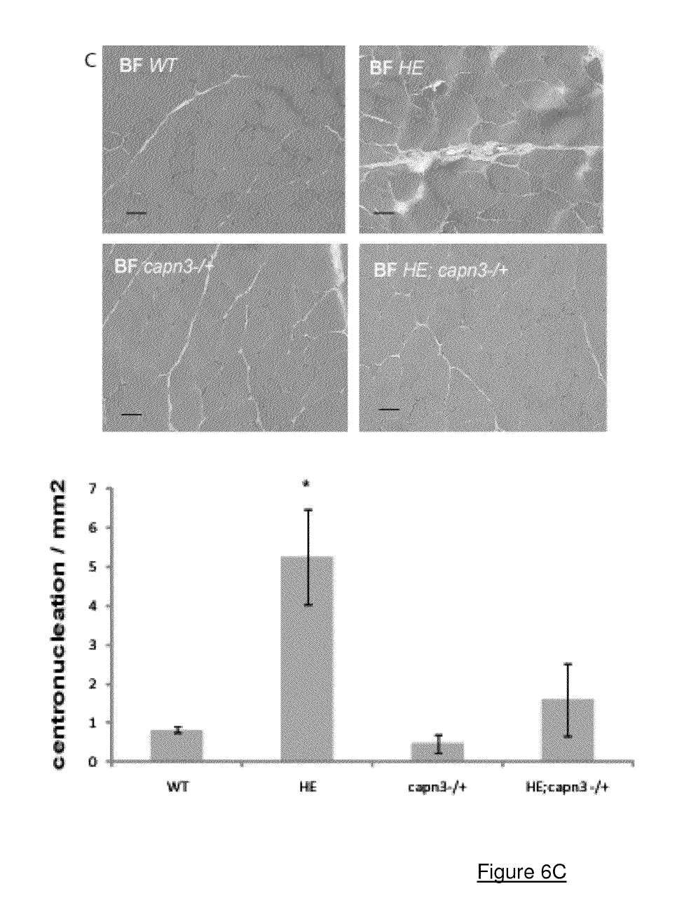

FIG. 6: Histology of the muscles of HE and WT, capn3+/− and HE/capn3+/− mice at 12 months of age and quantification of the centronucleation. A/TA B/QUAD C/BF. Bar=50 μm.

FIG. 7: Locating of potential targets for antisense sequences, located in the introns (lower case)/exons (capital letters) 2, 3, 4, 5, 7, 8, and 10, at:
(A) the branch sites (dark, with UMD score prediction) and the splicing donor and acceptor sites (light);
(B) the ESE sites predicted by “ESE rescue”;
(C) the ESE sites predicted by “ESE finder”.

FIG. 8: Position of oligonucleotides 1 to 6 (SEQ ID NOS: 3 to 8) capable of decreasing the expression of the calpain-3 messenger by RNA interference.

FIG. 9: RT-PCR on murine calpain 3 between exons 2 and 6 after 24 h transfection of the C3 minigene and of the AONs into a HER911 cell. The entire RNA transcribed and spliced from the minigene provides a 645 base pair band. The use of exon3-ESE2 and exon4-ESE1 AONs reveals the presence of a larger RNA band spliced by exon skipping. The arrows indicate the main isoforms after exon skipping.

FIG. 10: Test of the AONs combined on exon 3, 4, or 5 on cells 911 previously transfected with the calpain-3 minigene.

FIG. 11: Skipping of exons 3, 4, or 5 by the AONs alone or combined on endogenous calpain 3 in HER911 cells.

FIG. 12: RT-PCR of exon 2 to 5 (495 base pairs) performed on the RNAs of the TAGs (left TAs) injected with the AONs targeting exon 3; The TADs (right TAs) are the lateral controls of the experiment. In most TAGs, the visible quantity of calpain 3 is strongly decreased (arrow).

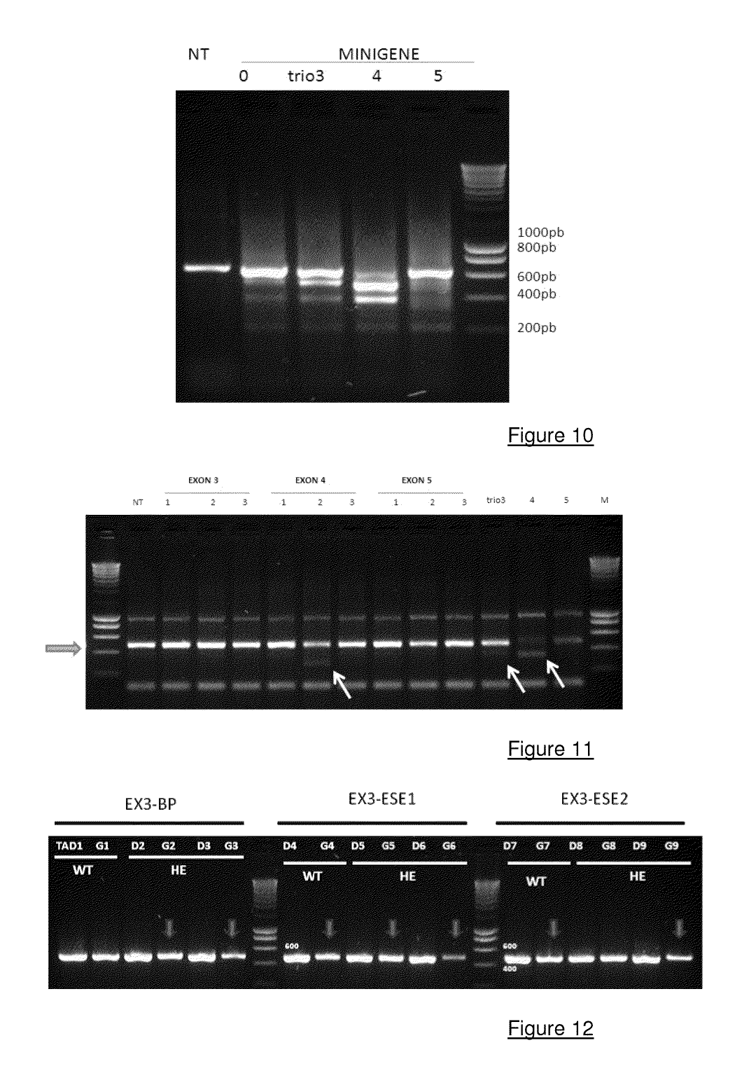

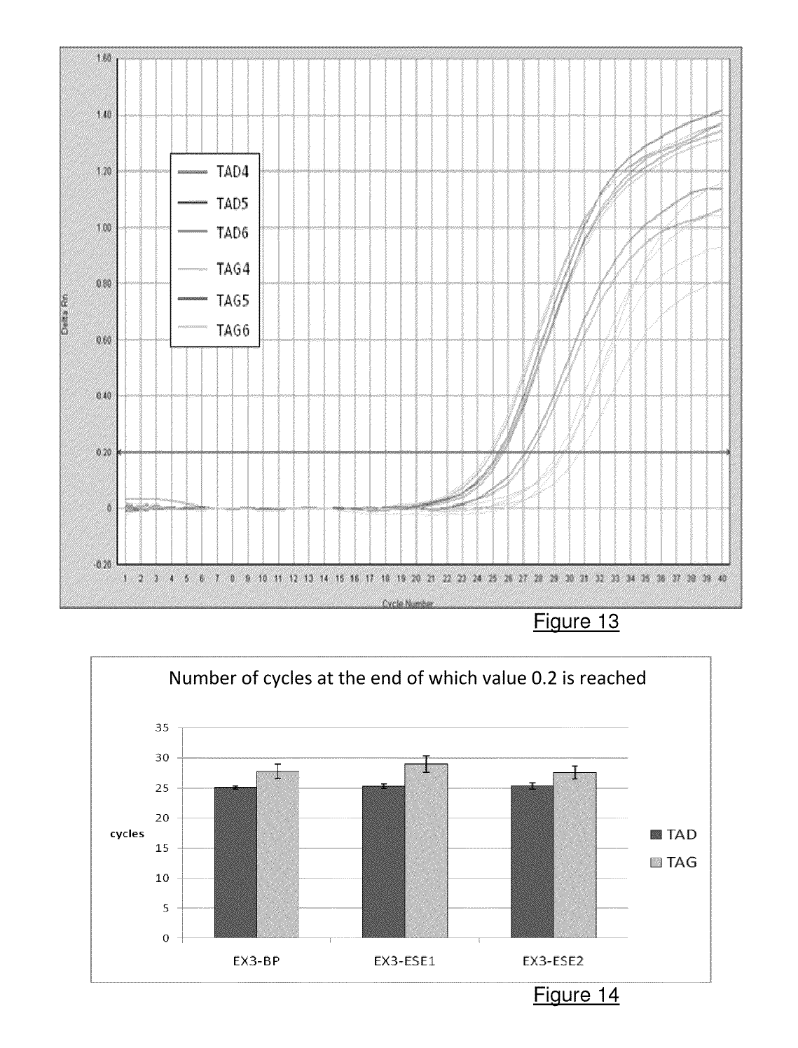

FIG. 13: Real-time RT-PCR of calpain 3 performed on the RNAs extracted from the TAGs injected with the AONs targeting exon 3; the TADs are the lateral controls of the experiment. Here, for clarity, only the mice injected with AON EX3-ESE1 are set forth. In all TAGs, the amplification curve of calpain 3 is offset rightwards. Each sample is presented in duplicate.

FIG. 14: Representation of the number of cycles after which the arbitrarily-selected 0.2 value is reached by RT-PCR.

### I) EQUIPMENT AND METHODS

### 1. Mice Generation and Genotyping

The targeting vector used for the generation of FINmaj knock-in mouse has been constructed at the “Clinique de la souris” Institute (ICS, France). A fragment of 2.2 kb containing exons Mex2 to Mex6 of titin has been amplified by PCR on 129S2/SvPas mouse genomic DNA with modified primers to introduce mutation GAAATAACATGG->GTGAAAGAAAAA into exon 6. The muted fragment has been subcloned in a vector containing a lox-neomycine resistance cassette. Two fragments of 2.8 kb and 3.6 kb (corresponding to homology arms 5′ and 3′, respectively) have been PCR-amplified on 129S2/SvPas mouse genomic DNA and directly subcloned upstream and downstream of the construction of the previous plasmide to generate the final construction. The plasmide sequence has been verified by restriction and all exons and exon-intron junctions have been sequenced.

The linearized construction has been electropored in embryonic stem (ES) cells of 129S2/SvPas mice and G418-resistant colonies have been isolated and amplified. The sequence of obtained clones has been validated after PCR amplification by using external primers, confirmed by Southern blot with external probes 5′ and 3′ and has enabled to identify a clone with a properly targeted allele. After caryogram, the ES transgenic clone has been injected into C57BL/6J blastocystes which have been reimplanted in foster mothers to generate chimera mice. The transmission to the germ line has been obtained after cross-breeding of the male chimerae with transgenic CMV-CRE females expressing the CRE recombinase under the CMV promoter, enabling the excision of the neo cassette. The Cre transgene has been eliminated by a first cross-breeding on a C57BL/6 background; the resulting heterozygous mice have then been back-crossed over 3 generations on a C57BL/6 background, and then cross-bred. All mice have been treated in accordance with the European Community Council directive of Nov. 24, 1986 (86/609/CEE).

The introduction of the mutation into the murine genome has been verified by sequencing of PCR fragments obtained by amplification of the tail DNA, isolated by using the REDExtract-N-PCR Amp™ tissue kit (Sigma), by means of the TTN 1180 primers located around the loxP site (SEQ ID NO: 13=GCTATCTGCACCTC AAAATCTGTGGGTTG) and TTN 1183 (SEQ ID NO: 14=GAACCCTGACCC TCTGGAAGAACATC). The resulting wild and muting type alleles correspond to PCR fragments of 414 and 502 base pairs, respectively.

The mouse model carrying both the FINmaj mutation and the muting calpain-3 allele has been obtained by cross-breeding of female mice with calpain-3 deficient male mice (Richard et al., 2000). The genotyping for the FINmaj and calpain-3 mutations has been performed on the tail DNA, respectively as described hereabove and by using calpain-3 primers (forward primers: GW 255 (SEQ ID NO: 15 32 AGTCTTCCTTCCAAAGTTGCCTGC) and GW 257 (SEQ ID NO: 16=GTGCTACTTCCATTTGTCACGTCC) and inverse primer GW 259 (SEQ ID NO: 17=ACTTCTCTGAAGCAAACTCCAGCC). The resulting wild and muting type alleles correspond to PCR fragments of 380 and 480 base pairs, respectively.

### 2. Histology, Immunohistochemistry and Morphometry

Cryosections (8- or 10-mm thickness) have been prepared from frozen skeletal and heart muscles. The transverse sections have been treated for hematoxylin phloxine saffron (HPS) colouring.

Colorimetric and immunomarking with laminine have been performed according to the ARK peroxydase protocol (Dako) to estimate the number and the minimum diameter of fibers. The antibodies used for such detections respectively are: a polyclonal anti-laminine antibody (Progen, P-4417, dilution 1:1000). The digital images of the colored sections have been acquired with a CCD camera (Sony) and a motor-driven microscope Nikon Eclipse E60 microscope stage. The images have been analyzed with software Ellix (Microvision, France).

### 3. Western Blot and Termination of the Calpain-3 Activity

The muscular tissue has been weighted and homogenized by means of an Ultra-Turrax T8 (IKA, Germany). An in vitro assay measuring the calpain-3 activity has then been performed on these extracts as described previously (Milic et al., 2007). For the Western blot detecting titin, the proteins have been extracted with the Subcellular ProteoExtract Proteome Kit (Spek, Calbiochem, Germany). In such experimental conditions, the Western blot of the fraction of the cytoskeleton by means of the M10 antibody reveals specific titin bands. The Western blots have been performed as described with 50 μg of proteins (Milic et al., 2007).

The proteolytic activity of calpain 3 has been analyzed by means of a polyclonal anti-calpain-3 rabbit antibody (Baghdiguian et al., 1999; dilution 1:150) and an anti-alpha-actin monoclonal mouse antibody (A4700, Sigma; dilution 1:500). The membranes have been incubated with secondary anti-mouse and anti-rabbit antibodies (1:10,000) coupled with IRDye® for revelation by infrared scanner Odyssey (LI-COR Biosciences, Nebraska, USA). The band quantification has been performed with software Odyssey 2,1 (LI-COR Biosciences). The titin band detection has been performed by using antibody M10. The membranes have been incubated with anti-mouse or anti-secondary rabbit antibodies (1:10,000) coupled with peroxydase (HRP; Amersham Biosciences, NJ, USA). The revelation has been performed with HRP Super Signal West Pico chemiluminescent substrate kit (Pierce, Ill., USA).

4. In Vivo Transfer Mediated by the AAV of the Complementary DNA of Calpain 3

Viral AAV2/1 adenovirus preparations have been generated by incorporating the AAV2-ITR-type recombining viral genomes in AAV1 capsides by using a plasmidic tri-transfection protocol such as described (Barton, 2006). Briefly, 293 HEK cells (60% confluent) have been co-transfected with pAAV-calpain3, plasmid RepCap (pLT-RCO2), and the adenoviral helper plasmid (pXX6) with a 1:1:2 ratio. The crude viral lysate is collected 60 hours after the transfection. To ease the salting-out of viral particles, the crude lysate is sequentially treated by four freeze-thaw cycles, digested by benzonase (15′ at 37° C.), and precipitated with ammonium sulfate. Finally, the viral lysate is purified by two CsCl ultracentrifugation cycles, followed by dialyses to eliminate the CsC. The viral load is determined by real-time PCR, as described in Fougerousse et al (2007).

The mice have received by intramuscular injection into the left Tibialis Anterior (TA) muscle 30 μl of AAVr2/1-calpain 3 (1.10^{e12} vg). One month after the injection, the mice are killed and the muscles are sampled and rapidly frozen in liquid nitrogen cooled with isopentane.

### II) RESULTS

### I. Characterization of the Murine Model Reproducing the FINmaj Mutation in the Heterozygous State

1) The Murine Model Carrying the FINmaj Mutation (Knock-In) Exhibits Late-Onset Local Muscular Damage

To study the TMD pathology, a murine knock-in of the FINmaj mutation (mouse KI TTN FINmaj) has been created at the Clinique de la Souris by homologous recombination. The mice (HE) heterozygous for the mutation have been characterized at different ages in parallel with healthy mice (WT). Although no general damage appears, certain muscles show, late, a centronucleation profile from 9 months of age, such as TA, QUAD (Quadriceps), and BF (Biceps Femoris).

The muscle histology has been performed on mice of 3, 6, 9, and 12 months of age and the muscles have been examined by colouring with hematoxyline/eosine and compared with those of WT mice (FIG. 1A). Finally, the calculation of the centronucleated fibers has been performed on these same muscles in comparison with WT mice (FIG. 1B).

2) Molecular Consequences of the FINmaj Mutation in Heterozygous Mice

A (non-significant) decrease tendency of the quantity of calpain-3 in heterozygous mice has been highlighted for the FINmaj mutation, after performing a Western Blot on proteins extracted from TAs (FIG. 2).

Finally, a marking of the M line of titin on a Western blot, enabling to see the degradation bands thereof after a subcellular muscular cell fractionation, has been performed. As indicated in Hackman et al.'s publication (Hackman et al., 2008), the loss of 50% of the titin marking has been evidenced by using an antibody targeting the C-terminal part of titin (FIG. 3).

### II. Modulation of the Expression of Calpain 3 in the Murine Model Reproducing the FINmaj Mutation in the Heterozygous State

1) The Injection of Calpain 3 Aggravates the Histological Damage of TMD Mice

Healthy heterozygous mice for the FINmaj mutation have received an intramuscular injection, due to an AAV encoding for human calpain 3, at 9 months of age in one of the TAs to see what effect the overexpression of calpain 3 could have on the histological profile of TA.

Such an overexpression has no effect on a healthy muscle. However, the muscles of mice heterozygous for the FINmaj mutation injected with AAV C3 have a much higher centronucleation rate than the non-injected control TA (FIGS. 4A and B).

2) A Decrease of the Quantity of Calpain 3 Improves the Muscular Phenotype of Mice Heterozygous for the FINmaj Mutation

The overexpression of calpain 3 in muscles of mice heterozygous for the FINmaj mutation tending to aggravate the muscular phenotype of knock-in mice for the Finnish mutation, the consequences of a decrease of the quantity of calpain 3 in these mice's muscles have been tested by crossing mice heterozygous for the mutation with calpain-3 deficient mice (KO calpain 3) (Richard et al., 2000) to obtain FINmaj heterozygous mice expressing 50% of the calpain 3 (capn3+/−). The muscles of the WT, HE, capn3+/−, and HE/capn3+/− mice have been analyzed in histology at 9 and 12 months of age. It should be noted that no histological damage can be observed on capn3+/− mice at these ages.

At 9 months of age, the histology of muscles TA, QUA, and BF (FIGS. 5A, B, and C) reached in HEs shows a profile with very few centronucleated fibers. The quantification of the centronucleated fibers shows a strong decrease thereof and a restoration close to that which can be observed in WT mice (n=3). The same could be observed in HE FINmaj; capn+/− mice. Indeed, such mice show a real improvement of the state of muscles TA, QUA, and BF (FIGS. 6A, B, and C).

3) Conclusion

As a conclusion, it has been shown that the decrease of the expression of calpain 3 in damaged muscles of mice heterozygous for the FINmaj mutation (in majority in Finland) and causing a tibial muscular dystrophy (TMD) in the human being, enables to restore and to decrease the dystrophic damage of muscles usually affected in such mice at 9 months of age (especially the quantity of centronucleated fibers).

### III. Identification of Calpain-3 Inhibitors

The desired inhibitor is intended to perform a calpain-3 exon skipping to partly block the translation of this protein, and thus decrease the quantity of calpain 3 in the muscle of mice heterozygous for the FINmaj mutation.

The antisense sequences capable of introducing exon skipping, to eliminate an exon critical for the proteolytic activity or of inducing an interruption of the reading frame at the beginning of a sequence are preferred candidates.

The targeted exons advantageously are exons 2, 3, 4, 5, 7, 8, and 10. At the level of the corresponding sequences, the important sequences for the splicing (branch point or BP, splicing donor and acceptor sites, splicing factor binding ESE (Exonic Sequence Enhancer) sites (SR proteins)) are preferred. They are illustrated in relation with the human gene of sequence SEQ ID NO: 1, in FIG. 7.

Since the experimentations are previously carried out on mice, the following experiments have been performed on the sequence of the murine calpain-3 gene. The selected murine sequences on exons 3, 4, and 5 are disclosed in Table 1:

TABLE 1
Calpain-3 specific oligonucleotides
2′omethyl tested for exon skipping in
mice.

| Targeted | | |
|---|---|---|
| exon | Name | Sequence |
| ex3 | ex3_BP | acagaucGuGAGCAcuccugc |
| | | (SEQ ID NO: 18) |
| | ex3_ESE1 | GGCuGCGAGAAACCAGCAGuCC |
| | | (SEQ ID NO: 19) |
| | ex3_ESE2 | uCCCuGCGuaguuuuCGAuGAA |
| | | (SEQ ID NO: 20) |
| ex4 | ex4_BP | auuacuGuuAGGAaauguguc |
| | | (SEQ ID NO: 21) |
| | ex4_ESE1 | ACGuuGGCAGGCAGuCAuCuAuA |
| | | (SEQ ID NO: 22) |
| | ex4_ESE2 | uCCAGAACuCAuuGCGGuGGuu |
| | | (SEQ ID NO: 23) |
| ex5 | ex5_BP | agaccaAuuuGGGgucacaga |
| | | (SEQ ID NO: 24) |
| | ex5_ESE1 | CuGuGAAGuCCuCCAuGGCCuCu |
| | | (SEQ ID NO: 25) |
| | ex5_ESE2 | uGGCuuuCuuCAuGAuCuuGuA |
| | | (SEQ ID NO: 26) |

The efficiency of the molecule tested as a calpain-3 inhibitor may be verified by the state of expression and/or of production of calpain 3, especially by means of the following well-known techniques:
transcript expression state by RT-PCR;
protein production state by antibody detection (Western blot or ELISA).

1) Proof of Principle of the Calpain-3 Exon Skipping on a Minigene

Preliminary assays aiming at assessing the possibility of performing the exon skipping on the calpain-3 RNA (exon 3, 4, or 5) have been carried out in vitro on a murine minigene formed of exons and introns 2 to 6.

To skip exons 3, 4, or 5, 2′omethyl chemical oligonucleotides (AONs) have been synthesized in company Eurogentec according to a PAGE purification. 3 AONs have been selected per exon (see table 1 hereabove).

After 24 h of transfection of the minigene in human retinoblast cells (HER911), by Fugene HD (Roche), the AONs have been added to the cells for 24 h by the same type of transfection. The cells have then been recovered and an RNA extraction followed by a reverse transcription has been carried out. A PCR between exons 2 and 6 has then been performed to determine whether the exons have been skipped on the minigene. The expected size of the minigene after splicing with the exon 2 to 6 PCR is 645 base pairs. bands are visible in the case of the presence of the minigene alone, which is the consequence of an internal exon skipping without the presence of AONs.

Possibles predicted sizes:

ex2+ex3+ex4+ex5+ex6=645 base pairs

ex2+ex4+ex5+ex6=526 base pairs

ex2+ex3+ex5+ex6=511 base pairs

ex2+ex3+ex4+ex6=476 base pairs

ex2+ex5+ex6=392 base pairs

ex2+ex3+ex6=342 base pairs

ex2+ex6=223 base pairs

On the minigene, the use of the exon3-ESE2 oligonucleotide causes a larger exon skipping of exon 3 (526 base pair visible band). The exon4-ESE1 AON also enables to obtain a larger skipping of exon 4 (FIG. 9).

The same AONs have been tested combined on the minigene of calpain 3 to improve the disclosed approach. The 3 AONs targeting exon 3 as well as those targeting exon 4 and 5 have been combined and transfected with the minigene, as previously. The exon 3 and exon 4 trios both cause a large exon skipping of the calpain-3 minigene (FIG. 10).

Finally, the skipping of such exons has been tested on the endogenous RNA of calpain 3 in HER911 cells which contain a small quantity of calpain-3 messenger RNA but not the corresponding protein. Again, the AONs have been tested alone or combined. The PCR has been performed between exons 2 and 5: expected size: 495 base pairs for the entire RNA.

Possibles predicted sizes:

Full size: ex2+ex3+ex4+ex5=495 base pairs

ex2+ex4+ex5=376 base pairs

ex2+ex3+ex5=361 base pairs

ex2 +ex5=242 base pairs

As appears in FIG. 11, the exon4-ESE2 AON causes the skipping of exons 3 and 4, causing a 242 base pair band, the exon 3 trio causes the skipping of exon 3 visible by the 376 base pair band and the exon 4 trio causes the skipping of exon 4 with a visible band at 376 base pairs.

2) Proof of Principle of the In Vivo Calpain-3 Exon Skipping in KI TTN FINmaj HE Mice.

The in vivo approach has been carried out by using the AONs targeting exon 3 only, mentioned previously (ex3-BP, ex3-ESE1-ex3-ESE2). 3 mM of each AON has been associated with the PEI and injected in IM in the left TA (TAG) for 2 consecutive days in WT (“wild-type”) mice heterozygous for the FINmaj mutation.

After 10 days, the muscles have been sampled with their lateral controls (TAD) and crushed to recover the messenger RNAs and the proteins.

A RT-PCR performed between exons 2 to 5 of calpain 3 enables to highlight in TAGs a strong decrease of the quantity of calpain 3, suggesting that the exon skipping has worked and has caused a large exon skipping (FIG. 12).

A real-time RT-PCR has then been performed by means of a Taqman probe to quantify the decrease of calpain 3 in TAG muscles with respect to their TAD lateral controls. The PCR amplification curves clearly show an amplification delay in the case of TAGs, as can for example be observed in the case of mice injected with AON EX3-ESE1 (FIG. 13).

Based on this RT-PCR, it is possible to extract the number of PCR cycles after which the (arbitrary) value of 0.2 is reached (FIG. 13: at the arrow level). Such values have been averaged group by group and are disclosed in FIG. 14. This number of cycles represents the original array value: the larger the number of cycles, the less calpain 3 is present in the muscle.

A Western Blot (WB) has been performed on the cellular extracts of this in vivo experiment. Calpain 3 is evidenced due to an antibody (12A2). The WB reveals the presence of calpain 3 at the 94 and 55-kD sizes, as well as the appearing of a 30-kD band in all TAGs injected with AONs. This indicates that a smaller protein results from the exon skipping, which protein does not comprise the domain coded by exon 3 and which corresponds to the catalytic domain of calpain 3. This protein is thus nonfunctional.

As a conclusion, these data show that the use of genetic calpain-3 inhibitors can thus enable to decrease the quantity of calpain 3 in vitro and in vivo. The in vivo proof of principle has been performed on WT and HE mice for the FINmaj mutation. This approach is thus possible to especially treat heterozygous TMD mutations.

### BIBLIOGRAPHY

Baghdiguian, S., Martin, M., Richard, I., Pons, F., Astier, C., Bourg, N., Hay, R. T., Chemaly, R., Halaby, G., Loiselet, J., Anderson, L. V., Lopez de Munain, A., Fardeau, M., Mangeat, P., Beckmann, J. S. and Lefranc, G. (1999) Calpain 3 deficiency is associated with myonuclear apoptosis and profound perturbation of the IkappaB alpha/NF-kappaB pathway in limb-girdle muscular dystrophy type 2A. Nat Med, 5, 503-511.
Bang, M. L., T. Centner, F. Fornoff, A. J. Geach, M. Gotthardt, M. McNabb, C. C. Witt, D. Labeit, C. C. Gregorio, H. Granzier, and S. Labeit. 2001. The complete gene sequence of titin, expression of an unusual approximately 700-kDa titin isoform, and its interaction with obscurin identify a novel Z-line to I-band linking system. Circ Res. 89:1065-72.
Bartoli, M., J. Poupiot, A. Goyenvalle, N. Perez, L. Garcia, O. Danos, and I. Richard. 2006. Noninvasive monitoring of therapeutic gene transfer in animal models of muscular dystrophies. Gene Ther. 13:20-8.
Clark, K. A., A. S. McElhinny, M. C. Beckerle, and C. C. Gregorio. 2002. Striated muscle cytoarchitecture: an intricate web of form and function. Annu Rev Cell Dev Biol. 18:637-706.
Dear, T. N., A. Moller, and T. Boehm. 1999. CAPN11: A calpain with high mRNA levels in testis and located on chromosome 6. Genomics. 59:243-7.
Diaz, B. G., T. Moldoveanu, M. J. Kuiper, R. L. Campbell, and P. L. Davies. 2004. Insertion sequence 1 of muscle-specific calpain, p94, acts as an internal propeptide. J Biol Chem. 279:27656-66.
Fougerousse, F., Barton, M., Poupiot, J., Arandel, L., Durand, M., Danos, O., Richard, I. 2007. Phenotypic correction of α-sarcoglycan deficiency by intra-arterial injection of a muscle specific AAV1 serotype vector. Molecular Therap
Furst, D. O., W. M. Obermann, and P. F. van der Ven. 1999. Structure and assembly of the sarcomereic M band. Rev Physiol Biochem Pharmacol. 138:163-202.
Furst, D. O., M. Osborn, R. Nave, and K. Weber. 1988. The organization of titin filaments in the half-sarcomeree revealed by monoclonal antibodies in immunoelectron microscopy: a map of ten nonrepetitive epitopes starting at the Z line extends close to the M line. J Cell Biol. 106:1563-72.
Gregorio, C. C., H. Granzier, H. Sorimachi, and S. Labeit. 1999. Muscle assembly: a titanic achievement? Curr Opin Cell Biol. 11:18-25.
Gregorio, C. C., K. Trombitas, T. Centner, B. Kolmerer, G. Stier, K. Kunke, K. Suzuki, F. Obermayr, B. Herrmann, H. Granzier, H. Sorimachi, and S. Labeit. 1998. The NH2 terminus of titin spans the Z-disc: its interaction with a novel 19-kD ligand (T-cap) is required for sarcomereic integrity. J Cell Biol. 143:1013-27.
Hackman, P., S. Marchand, J. Sarparanta, A. Vihola, I. Penisson-Besnier, B. Eymard, J. M. Pardal-Fernandez, H. Hammouda el, I. Richard, I. Illa, and B. Udd. 2008. Truncating mutations in C-terminal titin may cause more severe tibial muscular dystrophy (TMD). Neuromuscul Disord. 18:922-8.
Hackman, P., A. Vihola, H. Haravuori, S. Marchand, J. Sarparanta, J. De Seze, S. Labeit, C. Witt, L. Peltonen, I. Richard, and B. Udd. 2002. Tibial muscular dystrophy is a titinopathy caused by mutations in TTN, the gene encoding the giant skeletal-muscle protein titin. Am J Hum Genet. 71:492-500.
Haravuori, H., A. Vihola, V. Straub, M. Auranen, I. Richard, S. Marchand, T. Voit, S. Labeit, H. Somer, L. Peltonen, J. S. Beckmann, and B. Udd. 2001. Secondary calpain3 deficiency in 2q-linked muscular dystrophy: titin is the candidate gene. Neurology. 56:869-77.
Improta, S., J .K. Krueger, M. Gautel, R. A. Atkinson, J. F. Lefevre, S. Moulton, J. Trewhella, and A. Pastore. 1998. The assembly of immunoglobulin-like modules in titin: implications for muscle elasticity. J Mol Biol. 284:761-77.
Kinbara, K., S. Ishiura, S. Tomioka, H. Sorimachi, S. Y. Jeong, S. Amano, H. Kawasaki, B. Kolmerer, S. Kimura, S. Labeit, and K. Suzuki. 1998. Purification of native p94, a muscle-specific calpain, and characterization of its autolysis. Biochem J. 335 (Pt 3):589-96.
Kinbara, K., H. Sorimachi, S. Ishiura, and K. Suzuki. 1997. Muscle-specific calpain, p94, interacts with the extreme C-terminal region of connectin, a unique region flanked by two immunoglobulin C2 motifs. Arch Biochem Biophys. 342:99-107.
Labeit, S., and B. Kolmerer. 1995. Titins: giant proteins in charge of muscle ultrastructure and elasticity. Science. 270:293-6.
Lange, S., F. Xiang, A. Yakovenko, A. Vihola, P. Hackman, E. Rostkova, J. Kristensen, B. Brandmeier, G. Franzen, B. Hedberg, L. G. Gunnarsson, S. M. Hughes, S. Marchand, T. Sejersen, I. Richard, L. Edstrom, E. Ehler, B. Udd, and M. Gautel. 2005. The kinase domain of titin controls muscle gene expression and protein turnover. Science. 308:1599-603.
Machado, C., and D. J. Andrew. 2000. D-Titin: a giant protein with dual roles in chromosomes and muscles. J Cell Biol. 151:639-52.
Mille, A., Daniele, N., Lochmuller, H., Mora, M., Comi, G. P., Moggio, M., Noulet, F., Walter, M. C., Morandi, L., Poupiot, J., Roudaut, C., Bittner, R. E., Bartoli, M. and Richard, I. (2007) A third of LGMD2A biopsies have normal calpain 3 proteolytic activity as determined by an in vitro assay. Neuromuscul Disord, 17, 148-156.
Obermann, W. M., M. Gautel, K. Weber, and D. O. Furst. 1997. Molecular structure of the sarcomereic M band: mapping of titin and myosin binding domains in myomesin and the identification of a potential regulatory phosphorylation site in myomesin. Embo J. 16:211-20.
Partanen, J., V. Laulumaa, L. Paljarvi, K. Partanen, and A. Naukkarinen. 1994. Late onset foot-drop muscular dystrophy with rimmed vacuoles. J Neurol Sci. 125:158-67.
Pollazzon, M., T. Suominen, S. Penttila, A. Malandrini, M. A. Carluccio, M. Mondelli, A. Marozza, A. Federico, A. Renieri, P. Hackman, M. T. Dotti, and B. Udd. 2009. The first Italian family with tibial muscular dystrophy caused by a novel titin mutation. J Neurol.
Richard, I., O. Broux, V. Allamand, F. Fougerousse, N. Chiannilkulchai, N. Bourg, L. Brenguier, C. Devaud, P. Pasturaud, C. Roudaut, and et al. 1995. Mutations in the proteolytic enzyme calpain 3 cause limb-girdle muscular dystrophy type 2A. Cell. 81:27-40.
Richard, I., C. Roudaut, S. Marchand, S. Baghdiguian, M. Herasse, D. Stockholm, Y. Ono, L. Suel, N. Bourg, H. Sorimachi, G. Lefranc, M. Fardeau, A. Sebille, and J. S. Beckmann. 2000. Loss of calpain 3 proteolytic activity leads to muscular dystrophy and to apoptosis-associated IkappaBalpha/nuclear factor kappaB pathway perturbation in mice. J Cell Biol. 151:1583-90.
Sorimachi, H., S. Ohmi, Y. Emori, H. Kawasaki, T. C. Saido, S. Ohno, Y. Minami, and K. Suzuki. 1990. A novel member of the calcium-dependent cysteine protease family. Biol Chem Hoppe Seyler. 371 Supp1:171-6.
Sorimachi, H., N. Toyama-Sorimachi, T. C. Saido, H. Kawasaki, H. Sugita, M. Miyasaka, K. Arahata, S. Ishiura, and K. Suzuki. 1993. Muscle-specific calpain, p94, is degraded by autolysis immediately after translation, resulting in disappearance from muscle. J Biol Chem. 268:10593-605.
Squire, J. M. 1997. Architecture and function in the muscle sarcomeree. Curr Opin Struct Biol. 7:247-57.
Taveau, M., D. Stockholm, M. Spencer, and I. Richard. 2002. Quantification of splice variants using molecular beacon or scorpion primers. Anal Biochem. 305(2):227-35.
Taveau, M., N. Bourg, G. Sillon, C. Roudaut, M. Bartoli, and I. Richard. 2003. Calpain 3 is activated through autolysis within the active site and lyses sarcomereic and sarcolemmal components. Mol Cell Biol. 23:9127-35.
Trinick, J. 1994. Titin and nebulin: protein rulers in muscle? Trends Biochem Sci. 19:405-9.
Udd, B., H. Haravuori, H. Kalimo, J. Partanen, L. Pulkkinen, A. Paetau, L. Peltonen, and H. Somer. 1998. Tibial muscular dystrophy—from clinical description to linkage on chromosome 2q31. Neuromuscul Disord. 8:327-32.
Udd, B., J. Partanen, P. Halonen, B. Falck, L. Hakamies, H. Heikkila, S. Ingo, H. Kalimo, H. Kaariainen, V. Laulumaa, and et al. 1993. Tibial muscular dystrophy. Late adult-onset distal myopathy in 66 Finnish patients. Arch Neurol. 50:604-8.
Udd, B., J. Partanen, P. Halonen, H. Somer, B. Falck, L. Hakamies, H. Heikkila, S. Ingo, H. Kalimo, V. Laulumaa, and et al. 1992. [Peripheral myopathies in Finland—a new kind of muscular dystrophy in the leg]. Duodecim. 108:1331-8.
Udd, B., A. Vihola, J. Sarparanta, I. Richard, and P. Hackman. 2005. Titinopathies and extension of the M-line mutation phenotype beyond distal myopathy and LGMD2J. Neurology. 64:636-42.
Van den Bergh, P. Y., O. Bouquiaux, C. Verellen, S. Marchand, I. Richard, P. Hackman, and B. Udd. 2003. Tibial muscular dystrophy in a Belgian family. Ann Neurol. 54:248-51.
Wang, K., J. McClure, and A. Tu. 1979. Titin: major myofibrillar components of striated muscle. Proc Natl Acad Sci USA. 76:3698-702.

1. A method for treating muscular dystrophies or cardiomyopathies associated with an overexpression or an overactivation of calpain 3 comprising administering a calpain 3 inhibitor to a subject.

2. The method of claim 1, characterized in that the muscular dystrophy is tibial muscular dystrophy (TMD).

3. The method of claim 1, characterized in that the inhibitor inhibits the expression or the production of calpain 3.

4. The method of claim 3, characterized in that the inhibitor is selected from the group consisting of: antisense, silencing RNA, short hairpin RNA, and ribozymes.

5. The method of claim 4, characterized in that the antisense has a sequence selected from the group consisting of sequences SEQ ID NO: 18 to 26.

6. The method of claim 4, characterized in that the silencing RNA has a sequence selected from the group consisting of sequences SEQ ID NO: 3 to 8.

7. The method of claim 1, characterized in that the inhibitor inhibits the activity of calpain 3.

8. The method of claim 7, characterized in that the inhibitor is selected from the group consisting of: calpain-3 antibodies, chemical molecules, proteins, and peptides.

9. A method for identifying medical products for the treatment of muscular dystrophies or cardiomyopathies associated with an overexpression or an overactivation of calpain 3 comprising assessing the inhibiting power of a compound on calpain 3.

10. The method of claim 2, characterized in that the inhibitor inhibits the expression or the production of calpain 3.

11. The method of claim 10, characterized in that the inhibitor is selected from the group consisting of: antisense, silencing RNA, short hairpin RNA, and ribozymes.

12. The method of claim 11, characterized in that the antisense has a sequence selected from the group consisting of sequences SEQ ID NO: 18 to 26.

13. The method of claim 11, characterized in that the silencing RNA has a sequence selected from the group consisting of sequences SEQ ID NO: 3 to 8.

14. The method of claim 2, characterized in that the inhibitor inhibits the activity of calpain 3.

15. The method of claim 14, characterized in that the inhibitor is selected from the group consisting of: calpain-3 antibodies, chemical molecules, proteins, and peptides.

16. The method of claim 9, characterized in that the muscular dystrophy is tibial muscular dystrophy (TMD).


SEQUENCE LISTING (26 sequences)

SEQ ID NO: 1 - DNA, 54818 bp, human
gtttatatcagttagcctggtttcactatacagtacatcattttgcttaaagtcacagcttacgagaacctatcgatgatgttaagtgaggattttctctgctcaggtgcacttttttttttttttaagacggagtctctttctgtcacctgggctggagtgcagtggcgtgatctgggttcactacaacctctgcctcctgggttcaagcaattcttctgtctcagcctcccaagtagctgggattacaggcacccgccgccacacccggcttatttttgtatttttagtagagacagggtttcactattgttggccatgctggtctcgaactcctgacctcatgtgatccacccgcctcggcctcccaaagtgcagagattagagacgtgagccacatggcccagcaggaccactttttagcagattcagtcccagtgttcattttgtggatggggagagacaagaggtggcaaggtcaagtgtgcaggtagagacagggattttctcaaatgaggactctgctgagtagcattttccatgcagacatttccaatgagcgctgacccaagaacattctaaaaaagataccaaatctaacattgaataatgttctgatatcctaaaattttaggactaaaaatcatgttctctaaaattcacagaatatttttgtagaattcagtacctcccgttcaccctaactagcttttttgcaatattgttttccattcatttgatggccagtagttgggtggtctgtataactgcctactcaataacatgtcagcagttctcagcttctttccagtgttcaccttactcagatactcccttttcattttctggcaacaccagcacttcatggcaacagaaatgtccctagccaggttctctctctaccatgcagtctctcttgctctcatactcacagtgtttcttcacatctatttttagttttcctggctcaagcatcttcaggccactgaaacacaaccctcactctctttctctctccctctggcatgcatgctgctggtaggagacccccaagtcaacattgcttcagaaatcctttagcactcatttctcaggagaacttatggcttcagaatcacagctcggtttttaagatggacataacctgtacgaccttctgatgggctttcaactttgaactggatgtggacacttttctctcagatgacagaattactccaacttcccctttgcagttgcttcctttccttgaaggtagctgtatcttattttctttaaaaagctttttcttccaaagccacttgccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggtgagtagcttcctgcttgctggctgggttttccccccacggaggagtcctctcactcagcacctccggcagctcagctgtgcacatgggcactgggggaaggatcctggcagcagctctgctgggctctgtctttaagtgtgaagcagggaggagaggaacaggtctcagatatttcaccaaatctcagcaaaatccagagggagagcgcaggaggtggggctgattcttatgctctggctctttctctctgaaaaaaaaaaaaaaatcttgctttttataaaagtgggtggaactcagtttaattcatcctgtaaaaataaatattcctttctcagaacaaattccagacagcccagatgtacctgttcgttttaaatattattcatctttgtaagattatttcagtttctctggctaaaatcatgatgttattcttctttaatttaccaatggccatatctttctgaaacacagaaaccctagaaagagaagagtcataggcaaggaatattttttcatgcataaaatgttggggttaaagagagagagacctagcaatcgcttttgtccacctacctcacctcataagtgaggagtcaaggcacactagagtgaaatatatctagtgggcacatgacagagcccggattaaaactttctgttttaggaaactctcccagcctctgggtttcatttacagtgatcgccaggagggaaatcacattcccctggctcacctctctgatcatccctccagtgtgactcttgttcttaattcgagaaatatttattgagcatctactagtgccagcactgggcaagcaactggggggacagcagtgagtaagaaagaccaaaattccagctgtcttggaacctagggtcctgaagggaagatgggcattgaacaagagtgacattgtcaggagacgatgttctgggtgccacaggatcatgtggcaaggagagctaacctggtccagggagacaaaccctctctgaggaaatgatgacaagctgagacccaatactattgattagccatggttttctttaacctaaggtgggccaggcatggtggctcatgcctataaacccagcattttggaaggcccaggctggaggattgcttgagcccaagagttagagaccagcctgggcaacagggtgaaaacctatctcttttgtactaaaaattcaaaaaattatccaggcatggtggcacatgcctgtggtcctagctactcagaggctgaggtgggaagatcacttgaactcggggagttgaggcagcagtgagccgagatcatgccactgcactccaggctgggtgacaggagtgagacccagtctcaaacaacaacaataacaacaacaaacctgaggtggagtactagacaaccctgtatattccataggggctcttcttggttctatggtaaggcccataactgtcctgaaggccttcagtttagaagataacactactctgctatcagagtagttggacatgtcacaaccccctgctgaaatatcttcagtgacatccctctctcggcccccactgccttcaggacaagtccaaactccttagccaagccataggcccttctcagctggtccctgcctacctgtccaggcccatgtccatcctctttccccagatcctgacacaccagcagtgcttggacatttagctgctctgtagttccaagtcttctcacaccggaggcctttgctcatgctgtctgtctgcctgacatgccttttttgttcctatctgaacctctgctcaggtgtcacttccttcaatctttctcatgactgatttcctctcactgagagcagaaatccttcctgttcatctttgaggccccctcaccaagctcaggccagtcccatagtgggtactcagctgatgctggtgagtgaaaacccacgcccaagggccttgaccagtgagacaggataggcctgcgttcccaggcagggagttcctaaaagccctttcttgggccagacatggaggctcatgcctgtaatcctagcactttgggaggcagaggtgggagcatcaattgagctcaggagttggagaccagcctgggcaacatagtgagaccctatctctataaaaaaagtttaaaaaaataaaagctctttctcagaatggggaaaattaaaacctacaaatactgttttcagtgaaatcattggtggacttgagtcttcaagcgagacttacaaattaataaaaattctttaagggcctgttatgctctaggtactagattgggcagtgtccatgtttgaaatgaggggtgcagaagacttgtctgttctaaattgtgcccccaccaccaccatcaccaacaacaaagatcctggattttccactgtttgagtcactaaaggaccagataacaaaggccactcttggataaaatgcccacatgttcccctggggttaagatcacaggaacactgcccattgggttcactagcataaatgagaaatataaaattagagatggtcagagttggaatgattcttggagaggtgctccagtgtccttgctttataggtgattaaggcccagaaagggcaaatgacttatctacagccacacaagttgacttggaactgaaaaactgaaatgactccagaactttctgactgtaaagcacttcttggagacatcagttctccataaacttcttcagatgtagtgacctttgccagccctatgcagtctgaaaagactgtgtcctatcaggagaagcaccacacaggtgagtaaaggccttcctgcttttactgtctaaggcacagaacctttggaaaccaatctgaattttttttcgaccatcaagtattatttattataattacagtgataatgataataatgataaagggagaattattcctggccaactataattctagttaaaatagcatgctgcactttcagcctttccctcttaaaagaaaacaggctctggccgactgttcccagctcacctgtaatctcttctcttactcaccagtgctacctcacagactttcttcctctactcgggaacatcatgcattgagtttccagggcaaacaatctggttcctctagactttccttatatgatctatttctaggatcagaaagcaaacaaattattcaacacttctcaactgttaaggcaagttttcttgatgacagatcccagggcagaatagaatgggttctagaacagggcttctgggatgctggtgcccaatatactagggataggttggggatgggagggcaggtcgtgtttcattccatggggtggaaggttcaattgcaccacactcctggcctgtctccttcccatggtgggtttgtaaagagaacttgacctgtaaagcccagttcttggcttctgtctttctctgggaagcaagctgcctgcaagaagcatggcttctgctctctgccctccttgcagtaagttttctggaggcaagtccccatggggagtcatgcaggagtccctgggccacctggtaatgtgtggttaacagtaaagataatcgctaatcttgagtgagaactcaccacatgccagtaactaaatgctttccatatgttaattcatttaatcttcacaacaatcctgtgaggaagtaccattattgtcccactttacaaaggagggaactgagtctcagagggattaaataactcgcgccagatctcccagttagtgagggttggagctgggatttaaacccacacaatctggctctagagccccaaatctatctaattctgagatttagatgtcaatatccatttttattaaacaataaatgtgggattttggactcccacctgccctgcacttttgtagtattcctttattggctcaaaactcatgcatggaagtaaggtgtcatttattgggcctcctcaagacacaacaggcattgagtgacaccccaggggttgcccctgaaagcatcagcctctataagttggagatggaatggcacagtggctaagagcccaggctctgaggtcagataacctaagttcaagtctgggcctgctacctaacacttaggtaatcttggtcacatacttaccctcattttgccacaggttctccatgtgaaaaatggagaaaaaccataattgaagaacccatctcttaattaattgaggattaaatgagttaatgcaggctaaaggcttaagaccagcacatggttgagtgctcagtaaacatagactactcttattactgcctacaagcagctatgcagtctcttaaaagtggcaacatcataaacatttttccagcttaatttgtgcctatgaccagtcaagatattagctacagcaatgtgtttgagtacttcatgcctggtctaaatgcatccatccatctcagcatgaggatgtgtgtgtgaatgctcccctcatgggcaggggactgcttgtcctttagggcaattctagttatctacatcaaatataaaatgtacatttggttgtgaaaatctgagtgtatcatagagccaggaaggagactacaaaagagggatcacacttggctgggaaacttggcaccttgcccagtgattggctttaaaggctcactgagaccaacttgctgagcaagggcttcgtttttcccatctgcatgtgcctcagtcatgccaggtggagagttcttaagcttcttctctctcatctcctgcaattcccaaagcaggggcaacctagatccctgcttcttttggagggatgagaagtgaagggccagttgcaggaccttgtgatgaaaaggtatctggtcctctcctcacccttggtaacatgggatcccagaattgctccacagggccctgccctgcaccatcagctcagctggccagggcttccagcagtggaccctgacaacaatgaggttcctgggctcctcctgtctctggtgctaggtggagtcacctttctgtaaattccatgttattccagacccaccctaacctagagcttggagaaggaagggcccaccgtatttagcagctggattaacccagacttcaggttaaacttccctccatcagcatggtctcagtgtttcttggctctgctaggagaggttatttctggttgggcctccagttcacagccctgaatcggagtcaaaactgcatcattgatatacagccacctccagcttaacctccagctccttccctaggaaatccgttctcccttcctccactgtcctccaagctccttgggctgatagctgggccttgcagtttctcatcgcaaccttctgcttagctctctcctctatttgctgttgggagttgggggagcttgccaccttcccacttaactccagatccagtgccttctcacttgctgatggtggctggagctccagctatgggtcttttcccctcaccttttcccctcacctctttccctcaactcctgtccccacctctctgtcattacagggccccacccgttcccagctcagagctgggtctctacttcagccacatttctctggtgtttgcctttcttcctgccaggtattactatcatacccatatccatctgtgctttgcccagtagtccaacctcacccttagccctgctcttcttcactcctggggccagctagtgccctccaccaacacctggacctgtggacacctctttcccagacagacggcagcagcctcttagtcctctgaatagatgactttattccttgtattctggtacacagaaatctcttgaattaggcttattttaagaatagagtttagataagtttcccaagctataattcatcatgaaccttattttcccaagtctagaagattctcaatactagtactttcttgatcttatcagagggtgtccatggaaggtcatgaccatcacccagctgagtgcagtagaaggaagctttctaatgtaagttgcatcacagtttcagagatgtgagagaaattcagaaatgtgagggaaattgtgaaatgtaaaacgtgagggagaaaagcattcagaatggaaaaataagacaaagagtcccttcccatctaactcacaaccctgccatccccaacctattccttttcccaggctctttctttcttccgtgctgtctatcgacatggacccagccacaccggctggtttttgctgctttctgtatgacctggtctcttgccccttcccaaggtcctcatcatccaggcagaaccaacttcttccttgtgccagggctgttacaccaggagaaaaaaaagtatgacattcttcagggtagatcttcttactactgtggaattattgtcaagtctttaaatttatttcgccaaatgtgctcactttctgaacccttaacatggcagaaacatcttctctcttctcgaggaaaacctgtaagaaaaaatcagaaagattattataattacagaattacaataatctctggggtgggggaaggagaattaagtatgattgttttcgaggaagaaaagaaagaaaaacacctgcccgtttacttacaagagggaggagagctagaactgaaaggaaaatggagtggaggaagcaacagggcttgtgacgacccctaacctggtgttcttcctctccacgacccgaccctcacagaattcttttttttctttttttttttctttttttttttgagacagggtctcgctctgttgcccaggctggagtgcagtgtctccagctcagctcactgcaacctccacctcccaggttcaaccaatcctcccaccttggcctctctagttgctgggactacaggcatgcgccaccatgcctggctaatttttgtatttttagtagagatggggtttcaccatattgactaggctggtctcaaactcctgaccccagtgatccaccgcctcagcctcccaaagtgctgggattataggcatgaggctctgcacccggcctccctcacagaattctttccgagtgcttgtgaaacttctgaggcaagtgacttggtagagggtggaggttcctattttggtttacacaagcaggggtgagccaacactcagagtgtggaaagcacaggagtgtggggagagagaagcacacagaaaggagctgctaggaaccggctgaactgtggtgggagtgaggaggctctgccctctcagaggctatggagtggtgttcaagacaattcacaaggaagcctggtgcctcagctaacactgggtggcattctgctgaagaggagctgacacgtacttttccattttcattcaatcttcaagtttaaacaatcaccatcagaactttcagaagtgtattatatactccagaacaccagaaatatagtataaaagctgctatttttaaaataccccagagatataggaccaaaaatgggaactactttcagcctttccagatggatggctttctttcctgatataaaggttttaaagagaaaaggatggctccatctccttgagtaacctacagcttgtaactttcacagtctgaacactcatccttatttctaactagaatgtttccaggtgcaaatgtaatatattttctctggctgtgacccaaaagacagtgaaatagagtcagctgattttccctgtatggtaaccctccctatttttttctctcttttttttaaacagagataggagtctcactatgttgtccacactggacttgaactcctgggtttaagtgatcctcctgcctcagactctgagtagctgggactacaggcatatcctgtttttgaatcatgtcatccctcttttagttaattcagatcagtcacaaaagtgttccaactttatgccataggcatggcttttccaacatggaggacgtcagtgctataaaccttacacagccaatccttgttatttgcagatgccatatttgcaaattcacctactcgctaaggtttgtctcctcaaaattagtacttgaggtgtttttgcagtcatttgtgggcatacacagagctgtgaaaaatctgagtcaccccacatgcctgttcccagctcaggtcaaacaaggcaacactcagccttcttgtttcagctttgatactgtaaagaatgtccttttcatgacgtctttagtgccatgtttttctcatttctgtgctttttattggtgacctccaagcatagtgctgaaatgctgtctagcattctaggaggctgtggtgtgcctcatggaggaaatacgtgtgtcagataagctttgttcacacacaagttatagtcagttggctgtgagttcaatgttattgaatcatcaatcaataaacaataatttaatgaatcaatattaaataaggtgtctttaagcagaaacatcataaaataaggctctgtattgattggttgatgaaaatgtgaccagagactcataggactctaactctgtagttcccctaggagcaatggttcagtatttactaatacagtgttctcagcgttattataaaacataactaccaagaataatgagtattgactatacttcattttctctgtattcttaaatattacagcttgaaacaatgctctattaagaatgtgggctgggcgtggtggctcacacctgcagtcccagcattttgggaggccaaggcgggtggatcacttgagcccaggagttcaagaccagcttgggcaccatggtgaaaccctatctctaccaaaaatacaaagattagccaggtatggtggtttgtgcctatagtcctagctgtaagagttaaaaagaaagaggaaagaaacatgaaaaatggctcaataaagacaggtttattttggagaataaacctgagaggggcttctggctgatttctgtcaggagtactctctcttacagactaagtgtatttaagggttcagggtgagagagcctatcacaggcttggaatgtttctgtgtggaggagaagttcattgtgggtttggaatgtctctggttggaggggaggttatcttggggctggcttgtctctggtcagggagagagtttatcttacggttggaatgtttctggttggagatgtcatttgtggtttatggtcatactgacattagccattaggctgatgccctttgggttggatttaggcagtttttgatcaaggggcactttaaaatggtggtgcttgtccaagatggtgatgctcctgctctgtcactagctacttgggaggctgaggtgggaggatcacttgagctacggaggttggggctgcagtgagccaaaattgcgccactgcattccagcctgagtgacacagtaagaccatctcaaaaagaagaaaaaaaaaagaatgtgatcagtatgaagtattgcagcatgttttaatccattgcagtcattcttctttttcatgttcaaattgttccatctttggacagtaggagcccctgtatgttgcctgtatcatttcacgttgctccattaatcttttatccctttcttgttttctggccacaacaaaatgtgccacgcccatcttgtacatttgttgccccacaactggaatgaatcatttcttcaagaaacccagttcctttggtctgaaagggagggaaagaaagggcataagattgagaggacagatggccaccaacttccccagcacttggccatcgcatcagcactttctttctttctttcttttttttttttttttttgagacagagtctttactctcgttgcccaggctggagtgcaatagcgcgatcttggcttactgcaacatacatctcctgggttcaattcattctcctgcctcagcctcccaagtagctgagattacaggtgcccggcaccacgcttggctaatttttgtatttttagtagagatggggtttcaccatgttggccaggctggtctcgaactcctgacctcaggtgatccacctgcctcagcctcccaaggtgctgagattacaggcgtgagtcactgcgcctggtctcacatcagcactttcaagttggaagctttctgtccataccagacggtctcttgctctaatgacctcttccatgtgccccactgcctctaaaattaatactgactcctgtgtatcttctcagccaggagtcatttgacctgtgttactcagttctgaaaagaagaaatcctaggttggcacacaaaaaaaagttactccatgccagcatgagaagagagggagggggctgaatcacagaaaaacctaggctgtggcaggaagctctctgctcctagtgcctacgacccagttgtttcttctcccaggaaaattatatatctttccaatgaagctggctcagcgagtggtctgcttgtgatctgctttaagagtgttaccttctgtcagacagtttacagagaagtgaggtcactgtcctagccacagagaggaagaactgggggcattgtgcagagtggataaaacagctagagttagaaccaaacagacccggtctgaacactgactcggccactagcaagtcacataagtaagtcacattttttaagtttcgcttttctcttctgtaatgaggataaggatattcccatcatagggctgttttgagtggcattcagcgtcctagtaggtgtgcaaggtaaggtagcaattactattagcataggcagcaaagggtaggagcataacacaatgatgttatgacttcataacatcagagcacataacatcagagcacaggctctgaagtcaggcaaacttcattcaaatcccagctccaccgattaacgatgtgacctcaggcaaattattgaccattccgaggtctcagtttctgcatctgtgagaagaaaatgaaaattcctactttcatagagttgttgtaaggattaaatgagattctatacataacataatacaatgagtcaataaataatgatgatagtaactgaaagacaaaacgaggtacccatttgagagccagccttctgtctactcagcagagaatcatgatagtgtcatcacgggggtaggggggatggggaaggtgcatatgtttcagtgaaagtgacaattatataagtaacttttaggagcaacttctattatattctacaagacagcagctcctcctgggggtcatgatggggacagagctgtagacatcatggtgaacaaattaaagtgactcagcagtttaatgctgacaatgagaggggctgggacagaaatggccaagaaatacaagaaaagatgcattttcctaattataaataaggcaaagcatcttttttgtacacttgtctgttccttgatcatctgtatttcactgtacatgaaacacctgctaatgccttttgctgatttttctactgagttgtctttttcaatttgaattgtaggcattctttatatatcctggatactaatcctttcttggttagatgtgttacagataccttctcccaccttgtgacttgcctcttcactctctcaagaatacttttttaggctgggcgtggtggctcatacctgtaatcccagcactttgggaggccgagatgggcagatcacctgaggtcaggagttcgagaccagcccggccaatatgctgaaaccacatctctactaaaaatacaaaaattagctggacatagtggcgggagcctgtaatcccaactactcgggaggctgaggcaggagaatcgcttgaacccaggagacagaggttgcaatgagccaaaatcctagtactgcactctagcctgggtgacagagcaagactccatcttaaaaaaattaaaaaaaaatcttaaaaattaaaaaaaaaaaggctgttgggacttttattgcagttacattgaatcaatatgggaagaatacacatcttgacaatattgagtcttcctatccatgaagcagggatgtttctccatttaaataggtgtcttttaataaaagttttataattttctcagtgacatctttaacatttttttttctttgagatggagtttcgttcttgttgcccaggctggagtacaatggtgcaatctcggctcactgcaaacatcacctcccaggttcaagcaattctcctgcctcagcctcccaagtagctgggattataggcatgcaccataatgcctggctaattttgtatttttagtaaagacagaatttcaccatgttggtcaggctagtctcaaactcttgacctcaggtgaccccctacccttggcctcccaaagtgctgggatcacaggtgtgaaccactgcgcccagcctgccttgaacatcttttgtcagatttattcctaggcatcttttaacaaaaatggttaaacaaaagtcactttttttgttgttttttttgagtcaaggtctcattctttcacccaggcggtggctcacacgtgtaatcccaacactttgggaggccgaagcaggcggattacctgaagtcaggacttcaagactagcctggccaacatggtgaaacctcgtctctactaaaaatacaaaaattagccaggcgtgatggtgggtgcctgtaatcccagctacttgggaggctgaggcaggagaatcagttgaacccaggaggcggaggttgcagtgagccgagatcgtgccactgcactccagcttgggtgacagagggacactccatctcaacaacaaaaaaattattttaagctattatttaccctgtaatgccaaagtcatggtttcgaccagccacggtggctcacacctgcaatcccagcactttgggaggctgaaacgggcagatcacctgaggtcaggagttcaagaccagcctagccaatatggtgaaacccaatctctattaaaaatacaaaaattagccaggcatggtggtgtctgggtgcctgtaatcccagatatttgggaggctgaggcgggagaattgcttgaacccaggaggtggaggttgcagtgagccgagatcatgccaccgcactccagcctgggtgacagagcaagaccctatctcaaaaataaataaataaataagtaaagtcattggttttcaactttagtggacataagactctcctggtgagcctattcaaaatgcagagtcccagacccctaccaaatcctcaggtctgggatgggccctgggaatctgtattttcaactggcagaccagatgattctgatgcatgaagttcaaagatcacactttgagaagcacagagtagacaaaggccatcaggcattcatgagcagacagggagactgggtgtcctcctagcgggcaagctgccaggcctgagtttcctcccatgtcagctccctgctcttacattagagctgcctatttaagcacagtgaaagttgaaaaggctgggctttcttatgaatataattcccacatgacacacattcatctgctttccccatccaccacattctctgccctaaaggtcctgaatttgcaacagtggatatctgctcactgccactgggacctactctaacagggacttccatctgcagtcaagttacctaccctctgcatcattttcacagatgagagtccctaaatgactggtcaaggtctttgaggggtgggccagggacatcaggagtgtgaaggcgtcattacccccaaacaatggcatggagcagattcctcctatgcattcagtagcctgtataggattagtatatcagaaggccccatgctatagccctttgatcaccagtgtcaaaggcatgaacatcttttgctgagacaaagtaagcctctctgtaactatagtaatattttctttttagaggaagaatcccagcaaagggtgatgaaggttccctaaaggtcctcagaccactgcggtctgtcctgaggctgtccttggaactctctctcccctccctggctccctctcacagagttgcccctagaccacgggctcaggggcctccagtcaaagctgctgactctgaccatgcatggtgctgctgagggtggaagagctatattaggctccaggccattcacaagaggattaggcgtggactgagggtgcagtgggaaagaggcagcagggaaggcccagctctactcccagagcattcgccatgccctacctgctgtcaggattcttctgtgactgtgtgatccaagagagggacaggagaaatggagagggcaccatcgcacagcccctcaagtttgaggggcaggattttgtcgttctcaaagatgtctggcccagaagtgcctctttgaagattgtgtgttcctagcaggtgttcaggccctggcctcccatgagctgagccacaaaaccaagatttaggccatcacatggaaaaggccaaaggtagggatggaggggatgggacttcaggagccagctcttggggaacaagaagccaagtgatccagagcctgaggccatggatccctcctctgggcaattgacttatcaagggtctctttatccaaaatacctttctgctccttcctgaaactggagatgttattttctattcctgagctagagattattataatactttatgggtccctgaaatcgtaaatatttttagggagctaatgcattgtaatcgaatatcatgtctcttttttttttttttttttttttttgagagagagacttgctctgtcacccaggctggagtgcagtggcttgattatagctcactgcagcctcaaccccccaggcccaaacagtgatcccacttcacacctcagccttccaagtagctgggaccccaggcatgtgccaccacatttttatttgtttaaataaaaatttatttatttatttatttatttatttatttatttatttatttatttatagagatggggcctcactatgttgcccaggctggtctcaaacccctcggctcaagcaatcctcttgcctaggcctcccaaagtgctgggactacaggcaagatccatttcacctggtctcatgtgtctttttcagagcaggtgttttggggtgggagagtagagtggttagaagtactaaggctcacagtttccgggagagttctgtggaaggtggtgaacagtaaagaaatggaaggatggagacctgccttcttaatctttcacctaaggcttgttcattcatgtgctgtcgcttctgtttaaacagaaaggtccgtgctgagcagccagactccttacgggatgaagcagtttgtgtcaggctaccttatggcgcaaccgagaagccccttgtgtcaacttgagtgggtgaaagtgcttgggagtgtgtagcacaaggcccttcttcctgcttggagaggctcagagctccaaaaagggttcactgcatttagagcaggcaagaaactctctagaccagtaggctctgaggagggcatatacagcccagtagatctgcaagatgatccattggagtggatgaaaaaactattagaagtatttatatctattttttaaataaactgtaaatttcagaatggttttagatttacaggaaaaaattgtgaagatggtacagagttctcatataccttgcagccatttcttcctattaccaacatcttgcattagtacagtacatttgctacaattaaattagccagtgttaatatattattattagctgaagtccatacttttccttagttttttgtctgatgtcctttttctgttccaagatcccacccaagatatgacattacatttagtagtaacgtctccttagccttctcttggctgtgacagtatctctgccttctctggtgttttatgaccttcacagttttttaaagagtactcgtcaggtattctgtaacataatgaccctcaattggcatttgtctgcggtttttctccttatgggcttaagggtttgggggaggaagaccacagggtaaagtgccgtttttgtcacattatatatcaaggatgcgggcaataaacatgccttctcattgtggtgttgaccttggtcatgtggctgaagtagtgcttgtcaggtttctttactgtgaagttacttcttcccctcttttcatactatactctggaagcaagacactaagcataagcccatacttaagcaagggggaattgtgctctgccctcttgagggcagagaatctgcatcaattattgggaatcattctgcatgggagctttgtctattctccccatttgtttatttattcagtcacttgtttatatcaatatggactcatggatcttgatttttattttaagtcgtaatccaatactgctttattttgttgcttaggttgtcccagctttcttcactgggagctctcagttggctcctgtgtctatttaacataccctaattgtttgtgggtttttttttttttccttgagcactttcttactttctggcactaccagctcataatgtgtatctcctgccccaatcctagaatcagtcatttctccaaggagcactggtttcttttattgaaaaaggtattagatatcaagatctggttgctaagtctgctcattgctaccggagtgtgattgcttctgttcctctcagcttgtagagaaaggagatatgtgtgtgtactaagccagctgtatacacatatctataatatttctatgtataaccatctcacatatctgtaaatatttctatataaagccatttgtatctatactaagctaaacatgagttcacactgacataactaaatatttcttggcttagtgtgttaatataaaaggaatatttttaaatttcgaattccagttgttcattgctggtatatagaaaactaattggtgtttatatactaacttggaatcttgcaactgagctatactcacttattagttcccagtggatttttttgggtcatttgtttggaattttctacatagacaattgtgtcatctgtaaataaagatagttttttttttgtttccaatatttatatctttcatttctttttcttatcttgttgcattagctaggacttccagtaggacactgaataggagtggtacaggaggacatcttgctttgttcctagtcttaaggggaaagcatctagtttctcacccttaagtatgaagttagctgttattatttttgaaagtggatgatcgttaccaagttgaggacattctcctctcttcctagttagctgagaggtttttttaaatcatgaatgggtgtcagattttgtcaagggcattttctccatcaattaatatggttatataattgcttttcttagcctgttgatgtggtaaattacattaattgatttttgaatgttgaacaaaccttgtacctcaaataaatcccacttggtcatggtgtctgtttatacattgttgtatttgagttgctcatattttgttgaggattttgcttctatgttcataacagatattggtctgtagttttcctttattagagtatcttttactggcttgcatattaggataatgctggcctgatagaatgagttaggaagtgttccctctgcttctattttatggaagatattgtggaaaattcatattatttcttccttgaatatttggtagaattcattagtgaaaccatctggttctggtgccttcttttttggagggttattaattattgactcaatattcttgataggtatagtgctattcagattatctaatcctcgaatgaattttggtagcttttgtcattcaagaaatttgtcagtttcacctgttatcaaatttgtgagcatggagctgtccatatattcccttattatccctttaatgtccaaaagttcagtagtgattgcaggagtctgtcctgcagaccccagctcgtgcgacggatgaataacataccaagacaccaatatttagtgaaagagcagctaggggtccaagccgctcacagatactaaggaaggtgctgtgaagagtcagcagccacggccctgactagctagccctgtgggcatttatttagcacagttttaatgacaaaggctttgagtcaacacacctgtgggtaattaatctggttgcccccaccccggggggagctatcctgccgtgagtgatcaaaagttagttttaggaccacatgagtaaacaagctctttagataaactcctctacattcctatgtatctgtgccctaagctattaagtggattcggctgccttcagccaaacactttatgcaaacctctaggccttccaagagggtttgtgtttatatcctataaattcatcttaaaatttttgccaccagcctgactgaactcccaaaagtgataacctctccttcatttttaatattagtaatttgtgttttcatttttcttgtttagcatggctaaaggtttatcaattctattgatctttccaaagaaccatttttgttttcattgactttctattgttttcttgttttttatttcattcatttctgctctgattttttattgtttcttttcttctgcttgctttaggcttaagtatctcttctatagtgtactaagatggaatattgcttattgatttttcttttctttcttttctaacttatgcatttaatgctataactttccctttaaacactgcttttgttgcatcccacaaattttgatgttatatttttattttcacttagttaaaaacattttgtaagttcttttgaaatttcttctttgacccttgtgttatttagaactgtgttgtttggtttccaaataatttgggatttttcaactatctttttgttattgatttcgagtatcattccactgtggtctgtgaagacactgtaggctttctattcttttaaattggttgccggacgcagtggcgcacccctataatcctagcactttgggaagctgaagcaggtggatcatgaggtcaggagttcgagaccagcctgaccaacatgatgaaactccgtctctactaaaaatacaaaaattagccaggcatggtggcatgcatctgtaatcccagctactcaggaggctgaggcaggaaaatcgcttgaaccgggaggtagaggttgcagtgagccaagatcgcaccactgcactccagcctgggtgacagagagggactctgtctcaaaaaaaaaaaattggttaaggtgtgttttatggctcagaatgtgttctgtcttactgacttgttacctatgaatttgagaaaatggtttgttctgctattgttggatgtagaattctattaatgtcaatttgatccagttggttgatagtgttgttcaggtcaacaatatccttattgcttttctgcctgcctgcttgatctataattactgaaagagagtgttgacatcaccaactataatactggatttgtctatttctcctttcagttctatcactctttgcctcatgtattttgacactgttgttaggtgcacacacatttaagattgtatgtcttcttggaaaattgaccaattgaccttttcatctatatgtaatgcctctccttatctctgataattgtccttgttctgaagtctgcagtttctgaaattaatacaactccaactttcttttgattattgttagcatgatatatccttctttatctatgagtttttatatttaaagtgggtttcttggccaggcatggtggctcacgcctgtaatcccagcactttgggaggccaaggcgagcacatcacctgaggtcaggagtttgagaccagcctggccaacatggtgaaaccccatctctactaaaaatacaaaattagccgagcatgatggtgcatgcttgtaatcgcagctactcaggatgctgagatgggagaattgcttgaaaccgagaggcggagactgtgccattgcattccagcctgggcgacaagagcgaaactctgtctcaaaaaaataaataaagtgggtttcttgcagacaacatatagtcgagtcttgtttttatccactttcacagcctttgtcttttaattggtgtattttgatcattcacatgtaagtgattataatatagttagattactacctacatgtttataactgatttccatttattgcatttgttcttgtccctgcccctttttctggcttctctgatttaactgaaagttttatattttatcctattttatccttttacctcttcttttttgtctttttttttttttttttttttttttttttgagacagggtctggcttctgtcacccaggctggagtgcagtggtatgatcttggctcactgcaacctctgcctcttgagctcaagcaatcctcctatctcagcctcctgagtagctgggactacaggtgcatgccacaatgccaaactaatatgtttgtatgtgtgtgtgggtgtgtgtgtatgtgtgtatgtatatatatgtatgtgtatatatatatgtatgtatatgtgtgtgtatatatatatatatggccccattcttctcagcttcatggagtcttggcattcctgtgacacagttcaaacggggtctctccccccaccttccccatatatatatatatatatatatattttttttttttttttttttgtaaagatggggttttgccatgttgcccaggctggcctcaaactcctgagcttaagcagtctgcccaccttggcctcccaaagtgctgggattacaggtgtgaaccacggcacccggccttatctcctctcttggtgtatcaattatatttattttttcaattctagtggtcatgctagagtttccagtatacgttttcaaccaagtccaccctcaaataacactataccatttagtttgtactctaggtactttataacagagaacaaagtattaccatttccttcctttctttgcttatgacattactgtcatttgtttctcttatctgtatgctataattgcccaatacattgtttttattatgatttaactaagttaccctttagattaagaatttaaaatcaagattttattttatcttcatttatttatttcctctccagtgctcttcctttctttatgtgggtccaagttcctgacctgtatcatttttcttctccctaaagaacttcttttaacatttcttgcatggcaggtctgctggcaatgaatttcctctacttttgtttgtctgattaagctgcagaataacaatattaataatatcactaccaaaataattcctgaaaacagtttacagtctttccataggctttcttctttctcctttgatttaaaatggttttaaatagttgttcattgcccgaacactataggcaatatattacatactttacatcctccctttaatcatcaggtactttgttgggtttttgttttttctgagacagagtctcactctgtcgcccaagctggagtgcagtggtgcgatcttggctcactgcaacctctgccttcaggctcaagcaattctcctgcctcagcctcccgagtagctgggactacaggcatgagccaccatgcctggctaatttttgtatttttagtagagacgaggtttcgtcatgttggccaggctggtctcgaactcctggcctcgtgatccgcccacctcagcctcccaaaatgctgtgattacaggtgtgagtcattgcacccggccatcatgtaattttaaatctacaaataacgaaatactgactacttattctcagtccttatgttagtgtccttctatacctggttgtctgaaggttattctctagtatattcctcaggaagagctcgtgggaacaatattccctgaattcttatgtattagaaaagtttgcccacatcccttattcttgaaatgaataaaggaagtcatattgtatatacatccttggttcacattttctttccttgagcatcttaaatatactgctccaatttcttctggcataaaaaggccttgctactggaaagtctgataataatctaatgttttccccttaaaagtcacttgttctttctccctagatgtccaaacaatgtttttttttttctttgaatcccagtaattttctagaatgtatattcatgttggtcattctgggtcaatattcaatttgccctttcaatcaatatgtagtattaaatatgtgtttttatttaaggaaaaatattttcaaattatagtggggttttttgtttttgtttttgtttttttgagacagagcctcactctgtcgcccaggctggaatgcagtggtgcaatcttggctcacggcaatctctgcctcccaggttcaagcgattctctcacctcagcctcccaagtagctgggactataggcatgcgccaccatgtccaactaatccttttaatctttgtagagacggggtctcactatgttgcccagactggtctcgaactcctggcctcaaatgatcctcccaccttggcctctcaaagtgctgggattacaggtgtaggccaccacacctggacaatattattatttcttatggttcatatttcttagtatcttcataacattcaaggttcatatttctttatggttttggtttttgtccttaggatatattccaaaagggatagacagccaaaatgttacattttaagtgacttgaagtaattatttttctgtgaatggttatgcagttaggtccctttgcttcggtttgctttcagcttttacagatggctttttaaaataattttttctttaattttggctcttaattatttaaaactttacatgattcctaagtcaaaactatttttaaacagttatattcagagttctacttccatccctatttctaccaccttattccttccctccccatataggtaaacgtttttgaaaacgattgttttggtttatccttccattccactcagagttgcagatgggacgcatgctgtggggtgtagcataagcaagactttccatctactaggagtatacacccctgagtgcaggagcttcagctcacttgggtatattgggttgttgtgatgggggttgggggaaggtggagagagagaccctgtttgaactgtgtcacaggaatgccaagactccatgaagctgagaagaatggggctaaaaccagatcactagcctggcgcggtggctcacgcctgtaatcccagcactttgggaggccaaggctggcagatcacttgaggtcaggagttcgagaccagcctggccaacatggcgaaacctcatctctctctactaagaatatggaaaccacccatggtggcacacatctgtagtcccagctactcgggaggctgagacaggaaaattgcttgaacctgggaggcagagtttgcagtgagccgagatcgcgccactgtactccagcctgtgtcgcccactgcactccagcctggcgacacagtgagactctgtctcaaaaacaatacaaaacactgaatctatattttatttatttttattaatttattattattattattattttgattctcctgtctcagcctcccaagtagctgagattacctgtcaccacgcctggctaatttttgtatttttagtagagacagggtttcactatgttggccaggctggtcttgaactcctgacctcaagtgatccacccgccttggcctcccaaagtgctgagattacaggtgtgagccaccacgcccagccgacactgccctaactctcaagttgcatccttactcgaatagtatgacagtgtgggaagcagcatgggacaatgtaaaaaggaggcatgtttctggcttctgctacttactagctgtgtgtctttgcacgagtttcttaacctctctgggcctcagtttccttatctgaaaaataacaatgatagtattcccttcacagggccaaatggaatactatcaggaacactacataatggaactcaataaataatagctactgcggccgggcgcggtggctcacatctgtaatcccagcactttgggaggccgaggcgggtggatcacaaggtcaagagatggagaccatcctggccaacatggtgaaaccgtatctctactaaagatacaaaaattagctgggcatggtggcgcatgcctatagtcccagctactcgagaggctgaggcaggagaatcacttgaaccccggaggcagaggtttcagtgagccaagattgcaccagtgcactgcagcctggcgacagagtgagactccgtctcaaaaaaatacctatctatctatctgtctatctactgttattcttacctggtcatttcctttttgtttcacaggaaatttgcgagaatccccgatttatcattgatggagccaacagaactgacatctgtcaaggagagctaggtaggaaagtgcctcaggtcagatcctgccagatgatcaaggggtgattacaaggtgtgatccccttccaggaggtaaagggacaatctgtgcttgcttccagtaactttttggaagattttttataacagttgctttatggtcgtttatctacatgctggcgattgcttcatttcctcctacatgcctctttagcactctgccatgcatcacagggggtatctgcatcctgtggcctcctctccagtatctcaaggacacttacataccccactcagcatgacaaaagccctgcttttcactgtatcgtctttcttggaagacagctctgtgactgtgcaccaagcatgccccttgggcatggagattctagatacacacacaaaaggcatcgccaaggaaagcacttgtaactggaacccttgtttaaattgccccagcatagctccatcttaaaagagtctttccacaaagatggcatccgccatgtggatgagcatccaattttctctttgattggttagcttgactgctccatctgatcttcctctctctcgacctcttgttcagaaagtattgtctttggtgtggactataagcaagctctgtgaagtaaaattggagagaacaccaacagaaacaatttaaatttgaggaaaagggggcacctaagaccaaaggaatttggcttatttcattccagaaggggaggctgagaataaatcagatgaatatctgggttcctgcacctgagggaaggcttcctgcagagccctgggcataataatctgggaccttcaaaccaataacctctttccaaggaaagactggctgcttccaaggagggtaggggagagtggctgcaggcagctctcagtctcccctgcacactctcagttgcatttcacttaacccatcctccttagaaggcagctctgtgaccaggtacaccccctattatatatatatatacacacacagagagagagagagagagagagagagagaaagagagcaaagtgttacctccaactacatacagtactctgtcagaaaagaggttcagagaataagaaaacgtcccgagctcattccgttgccagcaatgtcttactgccccctatagacgggttccagggcagctgcctacctggccttccttccaatacaaatcatcttggtggatggttctctgaggctcagtcttcgctgaagtcagaagaggaattggactcacattgcaaaggcacagggcagggcagatttcctacaggtgttaggaagaacaacccagttatgatcacctactgctctgtctccattgaggcctaaaaaggaagtgagtttatactgcagttggaggaactgcctgcagccttgaggaaaatgtctagtcacaagggagtaagttacctgttgatcatattgtcaaggaattcctgtccaattctccttccctgggttgacacctctgtaaggtcagatctggaagtaggagagtgggcaccaagggagtccccgttcagggaagtggagtggctggctgggattggggctttttcttcccaggaggagcaggagtgctcacgatctgtgccctgtgtctgcctgcaggggactgctggtttctcgcagccattgcctgcctgaccctgaaccagcaccttcttttccgagtcataccccatgatcaaagtttcatcgaaaactacgcagggatcttccacttccaggtgaggtaatgagagtgtagttaagagggccagcggcaggccacccaccgctggtctcctggccttgacttcccagaagctggaggaaacttcccacccatctacccgcagcggcaacagtcggcatggacccccttaaggcttcaagcctgggaggaagcagttgcttatctctggctccctaatccctcccccaccaccttccactatgtcccagaaagacaggaagacatcctgtttactgtgggtctatttttgtctttgcagctgtctggctgcttttattgcctgcagcccttctcaagtaggtccctaagatattagcactgtgacaccacaggacccttcaggttgtacaggaacccctgtccagggctcctgtatacttcttcctctctaaggcatggcggtaccaaggctatcactcctctcttccaagccctggaagaagagtctgcttaacctggggatcaggcttcttgtttgccctagaactgaatctgatggttctagaatccatccagctactggaaattttctgggtcccagtcaccttggcatagagctggtgctagagcagaaccaaactgaattctacctgtgagggtctcgtagcttccgggatgctggggagtcagcctgtctccagcttcaaaggctccctcatgtcccaggatgacccacattatcagttcttgctccccgggtcttgcacctcagcacggaaggcctcagaaaaggtctgtctccaggctcagactccccctcctgccgccttgggaacatggcatatttaaagggtctcagatctaaagggccttacatacaaatatcagatagatttctgttctcatttcaatgagggagaaagtgccattgaaaaggagactaaaccacatttggcccttttcagttcaaactgattcattcaaaaaagagcgacatccaaacttgaaatgattgaacaatgttcctgctacagctagaatagattctgggtcactttgttcctccgtttcaatccttgttcttcagtttggcatcaagaaatacctaaatcagcacagtgccttcactgcatagttcccaatcctggccacattgaatcagctgggggcacctgagagtgctgacacccaggccctgccccagacctgctgagcaggagaatgaaaatcttacatcctaagacactcatggagcacctactctacccattactgggctggactctgtggaagacatgaagtatatgtaactcacttccagctctcaaaaagcacccagtccagttagagacagatttacacaccccaaacacaaaataggatgaacaggcacccagatgcagagtccaggaaatgatgctgctttgggattcaagaaccccctgaggaatgtggaggaaggacacatttcctaacagtaatttgagtatgtgactctgtgcgtgacgcttctgtgcagttctggcgctatggagagtgggtggacgtggttatagatgactgcctgccaacgtacaacaatcaactggttttcaccaagtccaaccaccgcaatgagttctggagtgctctgctggagaaggcttatgctaagtaagcaacactttagaatgtgaggtggggctagaggtgagaaagtgggttgcaaaatccagccgagacctcactcacaggaagaggcatgtgcctctatacgtgcatatgtgtgggcatgcaagtccaactgtgacccaaagttagagatcagttccaggcaacaacagctctaactaaaaacattaaatttaagagtagaaatgaagatttgcatagaagacctttagctttagcttaccatagcgagttctttcattgcacctccatggtggcattgcaagtcttggatcagagcattgtatctctggagctcagatcccagggtctcgcttggctcaacctcatgtgcttatagcagatttataaagccatgttgtctctcaacttaaaagctccaccccagatgctaataatggattttttaaatttttttgaaacaaggtctcactctgtaatgcaggctggagtgcagtggtgcagtcacggctcactgcagcattgacctcctgggttcaaggtgctcctcccacctcagcctcccaagtagctgggactacatgcgggcatcaccatgcccagttaatttttgtatttttttgtagagacggggttttgccatgttgcccagactggtctcgaactcttgggctcatgcaatccaccagccttggcctcccgaagcgctgggattgccggtgtgagccaccacaccggcagctgctaatggctttaatgcagcccttcctcaacgttcaggatgtagtggaaagagctctcaggaagtggggatagctgggtttcaatcccagtgcttctggctctctgtggtcttgggtgggtcacttagcctcttgagctcagtttcttcattatgaagaaagggaatcattgtttccatcccatgagctcatagggttaatgtggaattgatgaaagaacatcacagcatccaagaggtaaagttctggtggcagtggtacctgggttttgttccctggaactctgtgaccccaaattggtcttcatcctctctctaaggctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgtaagtctggggtgtggggcacagggtggggagctccaagtgtcaggaagccttttacccaatgaagggcagcatagagcttttgtgtgggacagagcgaatgttttgtttgaggaagcaggaactggctctcaactttgaggactgggaatttctcaagggagaacagttcttccggattttcaataaagacactggtcaaggacatttcaagccctggaatgtcagtggaaatcagtccagaggcctgtgtcagtggaggcctcccttgctggtgctcctcagtctcagcacgctcccattaagctggccacgtacttggctgtggacctgagcccaccatttccctaagaaagcctcccagtcactgggctttcaccacacctccccgcttgagacgtgggctttgtgttgttacctgggagaagctaagcctgcagcacctttcagtgcaaagaaatgctgtgaactgagacaggagccaagggtagggagatggccgcccatggccaggcctccttcagggggcatgccttccctgagggctgctcagtatattgatatgataatcttagtggtttccattggggaggatggggctgaagctgaattcctgccccttcttctcccaacacgcccaatggacagcttggaaggtcagttagcacacaacaccatggatgaactttttttctgtatcacttttctccgtctttcctccattcgtgctctgttgatctctcctctctccctttgtctgtcccatctctttctcctctctccttccctttccacccttctgtgtttgttctctccctcccctgtgttgttccctacattctccatcgggcctcaggatggcacgaacatgacctatggaacctctccttctggtctgaacatgggggagttgattgcacggatggtaaggaatatggataactcactgctccaggactcagacctcgaccccagaggctcagatgaaagaccgacccgggtgtgtacacctccgattatcagaactgaccatccctccaacccacatgaccccgccctattagtgtcagactcccctcagcagccagggccttacccacacacccccacctggcacctcccaagggtctgggttgaaataacttgctcagccaaggctcctgaagagggtgcaagaaccaggattttggagggaatctctgctggagtttctgcatattccatggtccaggcagttcctctcataacgaactatcagacagaaatacttgtaaagatacttcatttattttgaaatatttttcctcttctaatgtattcatttattcattcaacacttatttttgagctcctactatgttccaggcactcctctagcaaacaaagcaaattctctcctctttttcaatatttgtggaaaaagcaaggtctccctcttgtagagtttatattctagtattttcataagttatacctgctcactggagaatactgagccatacagaaaaacacagaggaaaatttcacttatatttttccccatgtaaagataaccactcttaacatctagtatatgttcttccaggatttttctatgcacacactgaatctgtatttttatttttaaaatgttatcatattgtatgtacctctttgcagcctgcttttttcagttagttttttttgtttttttgtttttttttttttttttggaaaccaagtcttgctctattccctaggctggagcacagtggtgccatctcggctcactgcaacctctgcctccaaagttaaactaattctcctgcctcagcctcccgacatagctgggattacaggcacacaccaccacacatggctaatttttgtattttttagtagagacggggtttcaccatgttggctggaatggtcttgaactcctgacctcaagtgatccacctgcctcagcctcccaaagtgctgggattacaggtgtaagccaccacacccggcctagtttgatattcttaatgtgcccaaagtattctcctgtaacattttttaatagctacacaatattcaaacacacagatatgttataatttatttacccaataccctattattggaaagttgagttcttttttttctttgttttgttttgttttgctactattctaaaatgctataacgaacatcccaatagatacatctttgtatacatccatggtgacttccataggacagattcccagcagtagaattgctgggttgaatgatatgcttaggtaatgacagaagagtcatttcaagcagcttcctaggtctagaactaaggattaatgagtctcccgccccctcccagtctattcagcatgatctggatcatgaggactgagatctggaagagactgagatctgggagaggctgagataccaaaagccctggctccacccatacccctcgccctgaaaacagctctaggaattccgcggcctagcaaggctccgggaagctccttttaaagctgttacgttagtaggcacatggaagccatagagagcctatccagggctcatgggactttagtgatcctgcccttctgccaaggatcccccatggctgcaacttggaaatttctgcaaatggaagagctactccttaggcacggtcatgtctgagcagggatctcctcgggctttcttagaattctctccctgggcactgggactcttgatttcttgaatattatgttccaggtgggtgtggaggaggtgaggggatgtaaagaaggctagacttggccaggcgcagtggctcatgcctgtaatcccagcactttgggaggctgaggcgggtggatcacctgaggtcaggagttcgagaccagcctggctaacatggtgaaaccccgtttctactaaaaatacaaaaaattagctgagcatggtggcacgtgcctgtaatcccagctactcgggaggctgaggcaggagaatcgctggaacacgggaggcagaggttgcagtgagccgagatcgcgccactgcactccagcctgggcgacacagcaagactctgtctcaaaaaacaaaaaagaaagaaaaaaagaaaaagctagacttacatgtgtcacttaaccccttttctcaacctctttctcttccaggaatagtcaaccctggatggcttcaggggaagggggatcctgaagcccagggcagcctccaactctaccccttcctcctttgaaggatactaaggggtccagaaaggaggggcaggacactgttacccaccccacatcccagcatccacattgctctctgatggtcaggacagagccttctcagggagaccagcctgtctggagctgtgtctcttggcactcttaaagggccactgaaggtccgttcgtggtcgtgaggcacactttcagggagcagagtggtctgtgtcttcacagagcccggaaaatgaactagtatgaactttgcctccaagcagcagaacttctgttcccccgcccctaatgggttctctggttactgctctacagacaatcattccggttcagtatgagacaagaatggcctgcgggctggtcagaggtcacgcctactctgtcacggggctggatgaggtaagcctggtggggcttggtggggcaagggcaccctcctgggttaacctcatgaagtcaggacttagctgttggggccccttccctgtctgcagagcttgcctccaatcaggacattcagttcaaggtccaagccacgcctgggagcagaggggcctgtgaaactggtagaggtggatcctgccacagttggtgcacagtttatctttgcttttcgtgctaaagatggcaatttttccaacatttccaatgaacaaattgaaatatcacttaactttgcttttacaaagttggtttcatgtgttcttgagcttcctgttctctcgtgttcagatagctacagttgtctctgggtagccacggggactggttccagaagccccaacagtaacaaaatctgcagatgctcaagtcccttctgtaaaatggagtagtatttgcatataacctatgcacatcctcccatatactttaagtcatctctggattacttacgatacctaacacaatggaaatgctatgtaaatagttattgcactgcattgggtttttttggtattattttctgttgttgtattattattttttctttttttgaatatttttgatccacaattggttatatgccaaagccatggatacgagaggctgactgttctgttttgctccttctgggacttctgggttttcctggaccatgtctgagacaggaacgttgtaagacctgttgcacacagttgggcaggttgtgccctgtacagagggatgggctgagaggggcagttgcctgcatcacccattgcagcagactggagggagtctgcttgtttgtagttcctcagtcagcaggggccttttgtctttctttctttcttttttttttttttttgagacggagtctcactctgttgcccaggctggagtgtagtggcacagtctcggctcactgcaatgtccgcctcctggattcaagcgattttcctgcctcagcctcctgagtagctgggattacaggcgcgtgtcaccatgcccagctaatttttgtatttttagtagagatgggggtttctccatgttgatcaggctggtctcgaactcctgacctcgtgatccgcccacctcggcctctcaaagtgctgggattacaggcgtgagccaccacgcctggccagcaggggccttttttctaatttatatgaagacacctaatttatatgtgttagcagggccctactgtttatgcctcacctcctcccccgaagctcataacggcaggatgttcctgagaaaattgcctcttagaagatagagaggagatggccaagccctaagttaggcagactcaggaggaaaggtctgacccaccccctgccattccccagcacacttgtgattaatctccttggccagagccaggcagaacaccctcgcgtaagagatttgccccccagccccgtcccagccctcagcaagacagaagattccctttccagagaggctgcagagcatgagagctctttctgtgtgcttaaggtcccgttcaaaggtgagaaagtgaagctggtgcggctgcggaatccgtggggccaggtggagtggaacggttcttggagtgataggtaggtgaggggaccccacgggattggcggtggcggggaacagggtccgggacaaggctgtgttgggaactgagccatgagagtattgaagatgcttggtataaaatcaccctcaaaaccaatgatccgcagagaagaggggcacaggtgttggctccagggaagggccaggagtggaagcggggtgctggggacccagagaggttgctgacaaccattggctggaaaggaaggattccagaaagcgtggggaaggtccaggcaggaaaagcgtatgaatgcagggttctgggctagagaagtgacttcccttcttggggtcttgtgttgcctttcctgtgaaatgggaacagtattattagcacttaccttgtgggctgatatgaggagtaaatgggactgtttttggcaagtgctgagccattgctaagattcccttacccgtgctgtccctgtataaaggcacaagggccctttgaaagaattttactgcttatcattgaaaggaatagactgggggcaacaaaaataacaaacatgcgaagttattatgaaggctccagcacatttgcaaaacctccatggtcctactggttcctgattacctccactgaatgagaggcaattcattactgaatgagccataagcgcctcttatttcgagagggggatggcaggactcagtagaggagaaggaccgcacccaggcagcctgggcccctcggctcctgtacttatatactgctgggtacttcttagcccagcatgtaattactggttcgttcagtcattcgtttagcaaatgtttcttgggcacctactacataggaggcacaggtcaaggcactggggatattctgtgaacccatagcctcccttgatacactgtgattaggggctgatcctcatgtcctgactgcctcctgcagacattctcatgccttccttcacctatgcacgctacacctatccttacttcctcagtgctgacttctgccaggccctaggctacccacctggggatgtagaaatgtagaaatgaacacagacacgtgggccctgtgcctgtgggtttaaggtccggggaaggacaggcaagcacacactaataattgagatgagtgccacagtaaggatcacaaagagggggcctctgcacctagtttgggtgggaggggaggcgtagagaaaggttacctggagggagtgacattggaactgtgctctgtgctctgggaaggccctcagtgactcctcagttgtctacttcactggcccaagtcacctgcagggacgcttgctgtgttctccagcctcagagcttggtgtgcagccctgccctgcttaccatgatgctgccatcacaggggctggagacaagtggacccaacgctgttcactcaaggagattccctttgctgcatccctcctcctggtctcaggaatctccatgtcccctgcagtgttctctgactgactccaagcgcttcctccaccccttacctttccagggagcacccagtttagttgaattctcctctgtgagcctgaaacacacatgcactttattgccaccatagtgaggagagagagtgtgaaaactgcccagcctttagaagcacaatgcttgattatttcccagcacaggaaaggagatgtttacagacttggcctttttccttagtaaatcagagtttcttattatatgacagacactatgctaacagctttgcatgcattattgcatctgcccatcccagccctgtgaacaggtgacataatcattttccttttgcaattgaggaagcagagtctgacagaggttaagtgacctgcccaaggttgcatagctagccctattattaaacggaactcccagtttgtctatgtccagaacccatttttttttttttttttttgagacggagtctcactctgccacccaggctggagtgcaatggcgcgatcttggctcactgcaacctccgcctcccgggttcaagtgattcttctgccttagcctcctgagtagctgagactataggtgcccgccaccacgcccagctaatttttgtatttttattaggacggggtttcaccatattggccaggctggtctcgaaatcctgaccttgtgatccgcccacctcggcctcccaaagtgctgggattacaggtgtgagccattgcgagcagcccagaactcaattcttaacctttaaagtatgatgaaaagaaggatcaagccctcaccagcccatttaaggagtttaggctcagtcttgaggatgtgagaagtcattgctattgggtttcacactgaggttaacaggtgaagtcagcattttggtagttcacagcagctgcaactctttgtatttctctgatacctcctgtcccaacctacatcaggccttcccttcttcctgcttccttaattcctccattttcccaccagatggaaggactggagctttgtggacaaagatgagaaggcccgtctgcagcaccaggtcactgaggatggagagttctggtgagtccagaacccaggaagacccagaagggtaagggtggggaagagaggggaaatctcagacctcagtccccagctaaggttatcagattccagcccttgggagatctgggctgtgttctcctccagcccaaggcccagcaaggatgaggttctgagaggagccttccaggccacagggacaactgagcccaggaccaggccaacatgacatggctcttgcctcctgtgtgcccctccgccacacactctattccagccacaggcaccctggccttagcacaattcttttctgagcctaggaagctccacttaccctgatcttccaacgtcaacctcaccctctctcaggttgtttctattcaggcttcaagtctcagcttaaggagaattttcaagtctcagcttaaggagagccccctaagttccccgaggactgggattaatttatgatgctcatcacccttaaaattgtttgcttaagccgggcgcggtggctcacgcctgtaatcccagcactttgggaggccgaggtgaacggatcacgaggtcaggagatcgagaacatcttggctaacacggtgaaaccctgtctgtactaaaaatacacaaaaaaagtagccgggcgtggcagcgtgcgcctgtagtcctagctgctggggaggctgaggcaggagaatcacttgaacctgggaggcagaggttacagtgagcccagattgcgccactgcactccagcctgggcgacaagagagactctgtcttggaaaaaaaaaaaaaaatgttgtcttagtataatgtcaagggaaaggttttttgtgtttttattactttattttttatttaaaaactataatagagacgggcctcgctatatttctcgggctggtctcaaactcctgggctcaagcggtcctcccaccttggcctcccaaaatgctggcatgtgggcctggtcaacatatgggaccccaactctacaaaaaattttaaaattagccagatgtggtggcgtgtgcctgtagtcccagctacttgggaggctgaagcagggggtcacttgagcccaggaggttgaggctgcagtgaactatgattgtaccactgcagtccatcctgggcaacagagcaagacactgtcttaaaaaaaaagtgctgggattacagatgtaagccaccacacctggccttctattttttatggatatatcatagctatatatatttttggggtacatatgtacccatattttgggggtacatatgtacccatattttgggggtacatatgatattttgatacacatatacaatgtgtaatgatcaaattagggtaattgagataccatcatctcaaatatgtatcttttctttgtgttgggaacattacaattcttttcctctagttattttgaaagatatataggctgggcgtggtggcttatgcctgtaatcccagcaccttgggaggctgaggcgggcagatcacttgaggtcagaagttcaagactagcctggtcaacatggtgaaaccccatctctacaaaaatacaaaaattagccaggtgtggtggtgtgcacctgtaatcccagctgctcagctggctgaggcaggagaatcccttgacctcaggaggtggaggttgcagtgagccaagatcatgccactgcactctagcctgggcaacagagcgagactctgtctcaaaaaatacacacacacacacacacacacacacacacacacacacacacacatatatatacacacatatatatacacacacatatacacacacacacgtctgtatatatatgtgtgtgtgtatatatacacacacacactatactataaattcttgtttagctatgtttgtcttactgtgctattgagcattagagcttttttttttttttttttttttgagacagagtctcactttgtcgcccaggctggcatacaatggcgcaatatcggctcactgcaacctccgcctcctgggttcaagtgattctcctgcctcagcctcccaagtaactaggattacaagtgcccgccataatgctcagctaatttttgtattttcagtagagatggggtttcaccatgttggccaagctggtctcaaactcctagcctcaggtgatccacctgcctcagcctcccaaagtgctgggattacaggcatgagccacagcaccctggtgagcactagagcttatttcttctatctaactgtatttttgtatccattagccaccctcttttcatcctcccctctccttcccttcccagcctctggtaaccactgtctgctctctacttccatgacatatgctttgttttagctctcacatatgagtgagagcatgcgacatttatctttctggccctggcacatttttgaatcattgttagaaaagatgatggtttggagtagatacatcagaagtgacagcgtttgccctaaaaaggaaagacaggctcctctgggaccctgaccaagttcctgtgaactattttattattgtgctgtgttagtcctggggtcttccgttcccagccctcctcacctgctcccatatggctctctctcttcttccaacctctcaggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccaggtgggagatgctcttgatggggggagggtctaagccgaaaaagttccaggcagaagaagcctaactagtgcttattaagtctctctgttccagacgtccactatcttattaaaccttccctgttttactgagaaggaaaccaccatgctgagaagtttgcaatagggagctggtagcaactttgaagcaggaaactgtgggaacaatgcagatgctgcttgacttacgatgaggttatgtccagataagcccatccatctttgaaaataccctaagtgaaaagtgcattcaatatgcctaacccaccaaacatcatagcttagcctggcctaccttaaacatgctcggaacactgaccttagcctaaagttgggcaaaatcatctaactcaaagcctattttacaagaaagtgttgaatatctcatgtaacttacttaatactgtactaaaagtgaaaaacagaatggttgtacgggtactcgaaatccagtttctactgaatgtgcatctctttcacattgtaaagttaaaaaattgtagccgaaccatcctaagtcagggactgtgagtactgtgtcagtaacagtaagggcactattggagaaccaagttagcagctgctgcaatagttcaagtcagagatgatgaaaacctagaccaagtcagtagcagcagagatggaggggagacagcagatttagggagagcatattgggtgatgtagggaaggaagaagaatgatgtcaagattcccagttggggacctgacaacattgcaacataagacacacaagaagatcgggtgggtggctcatgcctataatcccagcactttgggaggcagagccaggaggatcacttgagcccaggagttcaagaccagcacaggcaacatagtgacacctcatcgttacaaaaaataaaaaaaaaaatgaggtgggaggattgcttgagctcgggaggttgaggctacaataaactgtgatcatgccactgcactcctgcctgggtgacagagtgagaccctgcctcaaaaaaaaaagacacacaagagaaaaatatcagcgtgttgtttgtttttggtggagttaattgtggggttctagggaaaggaatttagcttgggacatggaaagtttgaggttcctgtagagtgtcccagtgaagatttgtaatagagcatcggatgcgcatattagatggcacttggtgatatgataagaactcaaaaaatatttgaggaataaaggaaagaagaggccagacgtggtggcttatgcctgtaatcccagcactttgggaggctgaggcaggcggatcacttgtggtcaggagttcgagaccagcttggctaacatggtgaaaacccatctctactaaagatacaaaaattaaccggggatgatggtgggtgcctgtaatcccagctacttgggaggctcagtcagaagaatcgcttgaacccaggaggcggaggctgcagtgagccgagatcgcgccactgcactctagcctgggcaacagagccagactccgtctcaaaaaaaaaaaagtgagagagattgaggctgggatatatggctcaggcatcatgcgcgtgtagggggcagttaaaaagcagaagtaagaaagattgcctagggaggcaggaagggtgaggtgagaggagaagaggcccaggaccagattctagtcaccaacagcgtttaaggggcaggtaaggaaaacaaaaccatcagcaaagactgagaatgaaagcccagagaggaaggaaaagccacacatacaatcagtacagctccatctgaataaaggtagcgccccccccccccccaaatcattagagaaatgcctgaatcgtgttttctgttgatatttcctaagcacctagatgtagggaatagaaataaatggctccctctgtctcatccccttcctgccctctgagaggcagctgtgaatgcgtgcttcctttctgggggtgcagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggtgtgcagtcctgattggctccagcccaggaaacatactttcccagggaggacgcttccaggggcttctagaggggccctctggcttcctcaatacccagtgacccacagagctcctggtatcaggaccacttgtgtttgtaacaagcaaaaaataccagggggggcattagagaggcagtggagcgggcctggcagaacaggtgcctgggggtcaggcttccgcatgcgggctgcagttgctggcattgccttccgcaggctcctcatcctcattcacatctgaagcatcttcctttctgtttcttctcaaggttcccaaagaggtatagcagcagcagcggccagcagttgtgtgcagcactacccagggggccccgagtctgtctgtggctcgtcgagaagcttcctggtggggtttgtgggcaggactgtgataggagagggccttgcctgtgttatttccactgcagagcaggtgcctcagggcattgcatgacccatgactaccacccccaggatgtgcactttctccctcgcaccagacactgcacgtcacacacatgcctttgcacactcaccctcctccacgcttacagccacacacacagtcacacagacgcgttctgagggtggctgcccgcttgggatggaggaatcacttccctcagaacccagccaagtcctctaggcctccttgggggtccttccagcctgaggggcttcggagctgaggagcagctgttctggtaagtgtccctgagtgtggggatgacacatttgccattcactctgaatcacaacagaaaaggaagaggaagtgaggtagggaggctatttaagccttgggagtcggaagtagggaggttgaaactgtgacatgggtgaccagggagttgggaagggacccttggaggtggctgtggcaggacaggatgttcctcccgaggggctcatgtgccctgggctctccccatctctcagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgagtgagtgctggcccagctttcccacgtgtttctaaaagctcacatggcccactccagaggttgaaggcatgaggcagctagacacgtctcctccagggtccttctgctgctcctgagccactggccacattacccccattcattcattcatccattctgtgatatttattgagcacctactatgttccaggcactgtcctaggcactaaggatagagtagtgaagtaaacagaaagaaatccctgccttcatggagcttaatattctaacatgagacaataatggataggaaaaacatatgtagcatgttagatttggagaggtgatatggagcaaaaataaagtagggaagagggataggaggtgttggggatgcttgaaattttaggttagcatggccaggaaagccacatcctgtccctggccaccacagatgagctcatagcccctgccactctgatctctgtccttggaagatgcaccaggtccatgggtaggtggctgggtcatgcctttggggggctctgagcaatactaacaagaacctgcgtgcctgggcttggctgtcggggatggtgctgacatggggctggttcctggggttggggtgttccaggggttctctagaggctggttctggcttggctgccaggaagccgtgcaccagagcaaaccgtccacgggcctcctgcttgcttctggtgacactgagaccccacatgtctgtattcctcacagggaagttgaaaataccatctccgtggatcggccagtggtgagtggtttagatcttctgtgcgaaaagtccagagggtccccttccctgaccatgcaggggacagatggtgcaggggagaatgggcactggcagagggaatgggagtctgggctgtgctgagcagtccctccttggcactgcaaatcctactttggcatggccagaagtaatcggccttaagcaccgggggccattgaggcagttcaggggctgggaaatatggaagagggtcctggaaaggagaagcaatttgaacaatcggagggaacaaggccacaggaagggatgacaagagccgcagcgaacactggattctgagactggataacattggatttcacacatagagaaaagaaagtaagctggtgccggacctggtgttgacacttggatcctccacttaccagcggggtgacctggacaatttctgtaatccctctcactcagtttcctactcagtaaaacggggatgataatgtgccttgcaaggcttttgtgaggcttcatcaatgaggtgatgtatgtgaagtgtctggcacagcatgggcactcaaacagaggtgctttttcacactttacaccttacaaggtacttttcacatgtgtcatcgcgatacttgcaaggttgctgagaggtagatggggttataatccctggtgttcaagaaaggaagcagaggctcaatggggttgaatgacttctctgagttcacagagctcagtaagtggcagggttggaactcacattcagactctctgactccagacttaggtttttccgcacctccacgctgaggccagccccaggcagtgagaagcccaaagtccgaagcacagagtgctgtgtgttgggctctgtgtgttgaggagtcttgtgactgccttggggctttgggctgtagtcagctgacagtcctttgtgctctgtggggatgacgtaggccaatgggaggacaaatgcccctctgaactgtcttctgggcagtgacagtcatggtcataatcctgaccctgagccagtgccaggtctccaagtgccttctgaatgaccacaggcgattggttttagtggtaggtgtgtggggatctgttctggtcatctggatgctggtcatcggtgtgcagtattgatcaggacctgcaaacccaaaagcttatgggagctggcacgtcacgtgagtagagcaggcaggtgcaggggttttgatgtccctgcactgacacagttgtctgcagttctcaattgacatttcgctccagtgtcgagggtcaacaggaatgttggggcgtggggcaatctggaagacacagggagcagggcccttggctcagctgatagttgccgcagggattacaggcccagggcagcctgccacagctggggcttttaccaaagaaaatctccctatgttaaatgcttgctcaaaaatttttaaaaaatattctgtaagtcaaaatccattgttaggtcagtttgagagagccatgtttttggtgttttagtaaccaatttcatttttttattatttatttatttgtttatttttgagacggagtttcactcttgtcacccaggctggagtgcaatggcatgatctcagctcactgcaacctccgcctcccgggttcaagcaattctcctgcctcagcctcctgagtagctgagattacaggtgcccaccatcacgcctggataatttttgtattttttagtcgagatggggtttcaccatgttggccaggatagtcctgaactactgacctcagataatccgcccacctcagcctcccaaagtgctgggattacaggcatgagccagcacgcccggccaccaatttcattttttaaaaaaggaagaaagaaaaccttagccagaagatctttttccttgccatatgcagtaagagtagattataaaaacaaagtcagagcagtcactggtgtctgggcatggaggagaaagaagaattctcttctcccttcaccctccatgcccctttttggctccatgtgattcagatttctggaccctggagccccaccccaagctaaagaccaggatacagggaagccaaaagccactggcggttctgagaacttacttttcacttattctgcatttactgtttccttttcttatgcagaaaaagaaaaaaaccaaggtaggtgtgtgggtagagagcatgaagtgtgtgtactcatgcatatgtatgtgcatgcatgtgaagtgtgcatgtgtgagctcatatgcatccatgcaccagacttgcctcttcctccccctccttcctgagcttctgctggggccgagcgtgcagtaatgacaactacgatttgctgggggaaggctacgtgccaagcactcttttaggtgctttccatgattaattccttcctcacaacagccctatgagattagtactataactatccccattttcagagggagaaaaggtacagacttgactaacttgcccaaggccacacagccagagaggggcagagccagtacttagagccaggcagtctgggtccagagtccgtgtcctgaaccacaagaggccatcatacaccatcagatttggtgctagcatttctggtggtgcctggtggtgatggatccatcacaggggtcctccaggtactggtgctggcccagaccagagctgacactcctcaggcactaccacattccaggcactgtgcttggggtcagtccctctcttttttttcccccccaattataacagtatctacaaagtaggtgctgttatttttcccctttcacaggtgagatagactcaaagaagtgaacttgcccaaggaacagaactaatgagtggggaaaatggaactggaaaccatgtctgtttactccaaaacctgtgtttcttgccctctttctctgatgccagccccctacacttcaaggcctgtgttgtccagacccacactcgggcctgccagtgtgtgcctggcagggatgctccatggccacaccatatccatcctacacatcccccctcagactgtgacctccatttgctctgggatccccacaagcttcagctgcttgagcaagacactgcttagaaggcagagcaagccaaggcctctggggcctgctgggagccaaagctggggagccgtttccacgggtctatctgcttgagctgtcctagatgagcagcatggaagggcagtggtgcatgagtccaggcgggctgcttttctgctccgagaggctctgcctgcccagttgttctctgcattgcagcctcaatccccacagccttgccttcccccggctttccctacaggtgcaccgcatccacagtgttggcaccatgcagcagccgctctccgtccttttcatatccttgtcacttgcacgagcatgtcttgaaaatatcccttgtttgtgtagcatcttaaatgtttttgcagtatgattttgcattcagtatctcatttgatccccacaagagccctatgaggagggaaagcagattttaccattaaaggatgagtaaactgaggccagagaggatatttttggttttttttgagacagtctcactctgtcacccagcctggagtgcagtggcttgatcttggctcactgcaagctccacctcccatgttcacaccattttcctgcctcagcctcccaagtagctgggactacaggcacccaccaccacacccagctaatttttttgtatctttagtagagatggggtttcacccagttagccaggatggtcttgatctcctgaccttgtgatctgcctgcttcggcctcctaaagtgctgggattacaggcgtgagccaccgtgcccggccagagaggatatttcttatatgaggggcagggctgggattccagcccagtgttctgatggctcacccactgaccatgccactaatccgtgtcctttttcaatctatactttgcagggttgtagaggttcctttgaggtgcctcagtagctgccatggtgatgtggggtgctgagggcaaagagctctgttctcattaatcagagaagcttgtgtttttatatacaccatgtttctgcaggaaatttaatttgaacagtgtttccatctggaaaaaaaaaagtctacaaaatacttgacaatcactgcactagatcatgctgcttttagcattcttagcatttcacgtgctgagctctcaatactctaccatgaggagggatggagtgggtatgaaaagataaagaactgaagtcacacggcttgtcagtggcagagatagagcttgaaccgaggttgaagagctcccgcctattcctttcctcttctcactggataaagctgctccaagagaggtgctgcctcagtgtgcctgttcagactgtaatcctcccttccttcctgcctcctccctcctctctccagcccatcatcttcgtttcggacagagcaaacagcaacaaggagctgggtgtggaccaggagtcagaggagggcaaaggcaaaacaagccctgataagcaaaagcagtccccacaggtgtctgggcatgtggcatgggtggggtggccagcacgctacaggggcttcctatgcgcttgggatacacaggggctggaggcttcccaggagtttgtcttgaacatctggaggtttgaatttgtcccactgaccttttctttcagcaagttcccctgaaatttgggctgctgcttgggtgaatatcccaggatgggggttccattctaggagtggactggcaggctgagcctcccatggagctgatccagccaggatacagagaaggggaggcaaaggctgagacagaaccagcttgagagcggaggcgcaactcttgtctcctggtggccttgagcatttcacaatagggggataaaggataggagcagaaaagtggggctgacttcagaaatggggtcctctagagctcacgggagggtgttagattggagtgggagcttagtggaggtgagccttagaggcaaaagtctccagaccaatccagggcccctcttctatccgggggcccctcttctatccagggcccctcttctgtctgggagcccctcttctatctggggcctcatgcagtggggcctaggggaggttctctgaggacttggccttgatgacagggtggctggaggaatcagaacggtcagaccttctttgacctgcgggcacctttagttggaatggtcaggcctgggatggtggagggggctcttgcaggtggggactggggtggcggggaggaggctgtatggccgccatatctcctttggctgggggcgtcagggctggagaggtgtgaagagtccctgaggcctcgatgcatctcactccagctcaccaggtctgcatttgcccgtccccagctcctgctgccacctccggccgttttaggcacttggctcccttggcccagaggagcttgcctcacaggcctgtgcacctctgacccctgtgaaccagttttcctttgtgcctccacagccacagcctggcagctctgatcaggaaagtgaggaacagcaacaattccggaacattttcaagcagatagcaggagatgtgagtacctccaagcccaggacgcccacaggtgcttccttctctcctggattaactgctcagattaccaattatttcattattgtttggtagaggtcactttggactttggtggagccaggggatgtgtgcgtagcacacaaatccacaagcccttgagttttggactgccacgtctgctggggggctcagaggcctttttgctctgagctgcccacggtggtcctgatagctgaggtgcagtatctggccccctgtcttcctcagaaaagccccagcttcccatgacataatagcaccgacagggattttacaaacacagccaggtggaatttgttttgcaaagtgtccgcgccaggagctgctgtactcctgaaccatgaccctcctctcccttcctcctcaggacatggagatctgtgcagatgagctcaagaaggtccttaacacagtcgtgaacaaacgtgagttgctcaaaccaaatgggggtggggtgggtggggagtcccgttgtctcaaagcagctcctcactcttctccatccccccagacaaggacctgaagacacacgggttcacactggagtcctgccgtagcatgattgcgctcatggatgtatccttcctgccgccccttcccgaccctctgtcatcagcccacgggggccaaggcaacatacagggtgcccagtcaggcaaagggccctaatttgtgcccagggaaacttaaggagaccctgattcagaacatcttggatactcgtctgaaaggggttgttagaggcggaaggggaggatgttgggttgtaactgccctaacccctgtgcttctctcaggcctgggatcctgcccaagcaaaagtggtccttaggagagcggctcctgggttacagagtaggcgcaatctctgactggtggtggagtggaggggagggttaaatagtacaacagggcagtgggtaggacagcccggagtctcctagaccctccctccaaatccagggggattttgctgtgtgctgtgtagccctgacctccctcctccagacagatggctctggaaagctcaacctgcaggagttccaccacctctggaacaagattaaggcctggcaggtgggaagagaaaatgaagcgtgggagtcaagaatggggttgatttggagattcagtgtgtgacctccatcctcaaattttctattgccagaaaattttcaaacactatgacacagaccagtccggcaccatcaacagctacgagatgcgaaatgcagtcaacgacgcaggtgctgagaaggaaggggtggcagggatgtggacccgagacggtgggagcaggaatgggaggggactaggctactagggccccactagagaaaggagagggaaagggcttctcactttcccttcccaggtcacagagtggccgagaggcagggaaaatagaaggcaggcccaaggcctccagctccacgtccacctctaacatggtcccctccacaggattccacctcaacaaccagctctatgacatcattaccatgcggtacgcagacaaacacatgaacatcgactttgacagtttcatctgctgcttcgttaggctggagggcatgttcagtaagtgggagaggggggctgccctctgctctcttgcaggggcagttgtggcaacaggcatctcacctgataatctccagtctgctccatccaggctgaacaagggccaatgacctctttaggcccagaatgggatggcaaagggagggttactggtgattctctgcctgcacatctttgtgctgatgagggacagcactgggcacatggtcctctgaggggaagttacagtagtagaggcggagtgcgcctgtaactggcctctggcctgtgcattctttcacaggagcttttcatgcatttgacaaggatggagatggtatcatcaagctcaacgttctggaggtaaagcataggcacagcacattccccctacacattaaaactcaaggtggaggggtcaacggggcggactggacccagggtgtgctcctcatttccacacagtggtggagggaagggataggaacagaacatggagggaggctcagcaggctcccaggacacatgcacttgaggcccaaaaggacctctgctcccccagtcacttgatgcgggaaaacatgcaccttcttagggaagatctaggagaaaggaaacagtaagccactgcttcttggaaaatcttctgggggtctgacctgctgggactgttccctttcctcttgccccgtaagattcctagggcggggggggggggggtcactcttttctgatctacattctgatcttgggacttctttcagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtccccttgtgttttgttgtctcacatttagatatcagccatgcatgactgaatggcttccaatcatatactcacctatcacctacaagagaacaatgaaaaacacacacaaaaacaaaatcttgaattttgtaatcatgcctattgctatttcttgagcataagaatggctcagatactttccaagacataaaaggaaggcagaggaatagttgttgctgtaaaagacatcaagaataaatgggtcatgtacaacgggaggggccggttacctgaataatggagtggagattgagctatcctagctcctctgctcactaactgacctgtcgcatgaccgtggacaaaaccctgaacgcagctgtttgtttgctaaacttctctggaccatggcctgcggcatatctataggcatcctgtgttttccacccagtttccttcttcctcgctaagccaacgtggaaagggctggccgtgaatatgcagacaaggtaacgaaagtaaaccgtcaattagtaaaagtactttattttcctcttgtatttgctttatatcttgctttacaaagttacgaagttcacagctttataccaaaatgtaagaaggctatttgcttataaacatttttgcagtcaggtgtcatctgatttcattcttctaatccatattcaatattaaaaaaatcagaaaccaagggtgctggagcagctctagggcatatatttctcttaaataggagaaagattttcaacagcttttcctccttgaccccctcctttcccaatttatttgggtcactaccttgaatttagagtgaatctgggaaatgtagtcaccaggtaacccagcagggccatatacttgggcttggaggtgaggttagaggttactgtctaatacagtaggtctggtttctatgaggagctcatatccacttgcataaggaaacctagtctatcccaaatcacctccaccacaacttctattcctgtctattagacca

SEQ ID NO: 2 - DNA, 43321 bp, murine
ccaacaacctgcccatgcctctacagcttttcctgcctgtgcctcccatctcactgtggatgctctggaatggcaaacattctactgcacctagttttaagtgggagctcaaatccctacacttacacagcaagtgctttaccactgagccatctttcctgccctatgcccccatttcagctgtcatctcccaccctttcctgtcttctaaacttggatctatagggatggtcacttaggaacaggcacagctgcaagctgggtcaagcctctactctgccagatatcagatatagccatttcattatgacccagagagcaactaccatggtaaaacaaaaacaaaaacaaaacaaacaaacaaacaaacaaaaacagaagccgggccatcagctccctcccccaggccactttggtttccatttaaaagccctgagtcttcactagacacgacatctaaggacttgacactaccctggctctatttccagttctgtctggatgctgatgtggtgtagctaaatgatctagtcaacaatctactaggatacacattatgtagtcgacttgatctcagtgtgtccagtgggcatgaagagaggaatttccttgactggaaattctgctgagtggcattttctacccagttggactgaaagccgactccagaacattctagaaaagatactggctctaaaatactgttttccatgtatccagcggctgctgcttcagtagcctgcatgactgcctagtcctttacacactttgctgatttctggtgttcagctgtctcagacactctcacttcttgacaagccgagttcttaatgtcaatgcagaaatacctccagctaggtccttcgcgaccactcagtctagccacgcaccagtctgtggctttcatgatcacaacctcacttcatgtctatttttagttttcctggcttaagcttctacaggccactgaaacacaaccctcactctcttcttctccctctggcaagtgcgctgctgaaggccactaggtagaaactgcgtctaaatcctttattgactcttcctcaggaaaacctatcgctctagggtcatagttcactatttgagctggtcagaagccagtcagttttctgataggatttaaactttgaagagactgtggccatttttttcctcagatgacagaatcacttcaacttcagctttgtaatcacttcctttccttgaaggtagctgaatctcattttctttaaaaagtcttccttccaaagttgcctgccatgccaactgttattagtccaactgtggccccaaggacgggggctgagcccaggtccccagggccagttcctcacccagctcaaggcaagaccaccgaggctggaggtggacacccaagtggcatctattcggccatcattagccgcaattttccgatcatcggtgtgaaagagaagacatttgagcagctccgcaggaagtgcctagagaagaaagttctttatctagaccccgagttcccaccggatgagacctctctcttttacagtcagaagttccccatccagttcgtctggaagagacctccggtgagtagcttactggttttgtagctggctggagtttgcttcagagacgtcctcccactcagcacctccccaagcacagctgggcacatggacacggggaaggatcctggcaggggccagggctctggctgcaagtgcaaaaaggagaggacagggtcaagtctaagaaagtctaagaggacagggtcagggctgcttctctcacactttctcgctttctgctgttcatgaaagtaattagatcctaaatttatctttaaaatggccatcctttccccagaatcagtgccacggtggccagagctatactctctgcctcactatattgagacgtcttccatttctcaggctaaaatcatgagatgctgttctttttgttttggacatttgccagggaccgtggatttctgaaacacagaagccagggaaagaggagagaggcaaagactactttttataacataacattgagactagaagaatttttgcctttatacctcactgcataaattaggatgccaaaacccagcaatagactgagcatatgacagaacctgaatgaaaccctgcttgctagggacccaccagaagggaaattatgtttccctggctcccttctctcatagtgcctccaatgtgactcttgtttttgaaacaagaaacattttagtcacaaaacaatctggggctaggagtaacacttagtggtagagttctacttagcatgtcagaggccgcttgtgcaatctctggcactgctaaccagtcagtaaataaacataaaaaggggaagagagaaaaaataatcacagaaaagagggagaaatagatgttgcaggtccactagcttcacatactgggcaagttactaggaggagtgttgtgaacaagagaccacaacttacactgttgcgaagactagagtcttgaggagaatgtggacatttaggtccagtggtcaccaggactctgtcatcacagagtggatgggatcaaagagagcaggccaggtcaggggtgagaaacctttcacaaagaagtgatgctgggctcttcacaggattcattggccatgtttttctctatttactaactagagctgtctcctgtccccttctgctgtcctttggtaagtcccatccatggctgtcatgaatgtcctcagcttagaagagcaggctgcagtaggctgggtgtagctcagcacacacctaacatgtgtgaggtcccaagccaaggtggacaacccaagcaatgcgcttagacacactgtgcccccccaccccgctcagcccccactacctgcagcataagttcaaactgcctcactgagcacagcccttctcaagcagacccgtgcctgacctctcaccttcactctcttctctaaatgctgtagcggaacatctggctgcagtggaacatctggctgcagtgccctactagggtttggttgattggctctgccttgcattctctacttcaatgtcacttccctcaacttctctcacctctcctggtgctgagagcagggtctgtttgtggtcattcgtagaccctgtacatattctgaacataataagatacagatgctggtagcaaatgaatacccacatctaagggtctacccaggacagccctgccttccaaagtagggaacccctagaagctttgtctaattgtactcaaggggcaaagggaccaaaacctacaaacactgtttttactaaaatcactggtggctttgaacctaaaagctagatttgcaaatctgcaatgttctgtggcagtgactggtctatgagctcatatggaccagtatgctccaagagacctggctgtttgatattcttcccctctcaccaccaccaacaacaaagatcctgaattttcaaaccatcttggttaaaatgccatttgtccctccatggttaagatcagtgggttggagagatgactcagtgattaagagcacttcctgctcttgaagagaacgtgtgtttggttcccagcacctacatgtggtggctcacagctgtgagactccaattcccagggatccagtgcctcttgtgatctctgcaggcaccagcacacattcatgcactcaggcacacatacatatgtacatataaaattaaaggtcaggacatacttgtattaacattctaaaatgggtttagaggtgggcagagttggaacgggtcagggaagtgttatccaaacccttcgctttacatgattatgacccagaaagtgcaaagaacttatgtatattgaaatggctccagaactttctaaaatataacccttcttggaaacatttgttctcccaaacatttccttggatatagtaacctctgtcaggccttagagtatgaagaagcatgtcctatgaggaaaaggaggagatgagggcttcgtttcgctggcatatctactccctgctgcctgctgttagtgttttaagcccacatccagctaaatttgctctgcccaactgtctaaccttcactccttcacctcgctagttccctagctgctgacacctgtgcctgaaggaagcctccagtcagacagaagcccctcagtcctccattcaggagacctactgcttattaccataactggacatttcttgaattaggctccaaaggtgacgtttaaatgagtttcctaagctgccactaatcaacaaccatgtgttaccgagttgaggtgttcagcctagcattgcttgatctccaagtccatagcgggtaatggccatcccctcaccccaccaagcactttggaaggaagcctggtggtgtaagatcaactgtggcttcagcactgtggagggaaatatgagggaagtactgcagtgggtgggaaaagagactcatgagactcttgcccaccagcatgaattcagccaccttggctagattctgctgctctctgtgtgacctgcaggcttcctgccatccttatgccccttccaggatcttcactatctgggctgaagtaactcatccccagccccaaggcagttattacaggaatgcaaacaacagctaaatacactctgggcatcaggagtcttgttcctgagagaatttgtctgattgtttgcatgtctttagcagatgtacttcctctctgaatccttaccatagcagaaatgttctgtcaagaggaaacacaaatgggagaatgagagagagggagaaagagagagggagagagagggagaaagagagagagagagagagagagagagagagagagagagagagagtgtgtgtgtaatcccataactataggttcatgcagagtccagaagaaggcagctgaagatggactcactggctgttgctgttgtaagctgcctgataactgaacacacatcctttgcaaaagtagtatgtgctctttaacctatgagccatctctccagcctgcattataattacataaagagtacttgagccaggctcgtgggtacctgcctactactaccgagggggcttcctagctactgaggaagctgaggcaggagggctgctgacactctcaagtttgaggccaacctgggcaatatgaggatcttgtcacttaaatacaaacaaaaacaagttaacaacaacaaaatacagggtgctttttttttttttttttttggtttttggtttttggttttttgagacaaggtttctctgtgtagtcctggcagtcctggaactcactttgtagaccaggctggcctcaaactcagaaatctgcctgcctctgcctcccaagtgctgggatcaaaggtgtgcgccaccactgcccagcctgagtgcttttcttaaaggaggcaaacatgattgttttctaaagggaaaaaggaaagaaaaggatcagctggttgatatataacaagagtgaaaggtagactgcactggaattggactagggaaagcctgaggtgcatgcacacacacacacacacacacacacacacacactgctcctgccattctctatggcctaacagccactgaagctgtgaaatacttgagaccagtaaccttttgggaaagggcagacttgctctgcatcctgtcttgaagtggcagtgctgagccagcttgccagtgttagaaagctctggaatggagaaaaacaaagtgagagaaaatgcccaggcttcactgagctctggagtgggggagacagaggaggagaccctgccactgagaggctgtgggttgaagacctgtgtgagtctccatggatgccccaggacctctcacacttgggaagcatcctggccactccacagtgccactcagtacagcatgaaaactgctattaaacactccatgtaagtccaggagcaaaatgttgaagaacaactgtcaaaggtctgaggtagttggctttcctcctgacatgaagagtcagaagggcaaaggagttctccttccacacacacatgctgtaatttccatattggaaagcttttcatgtctagcgggaagtcatctggttacaacccagacaacagtaaaggtatccagtggtgtcctctgttcctcggttgtcaccctatctctggttgctttatcagatagtcacaaaacgcttgggcaggggtttatccagctcaggccattagagtcctcaactgcacagccaagcctcattatctagattctatacttgttattctcctacttaaattcattggtaacttccaaatcaatgctgtgcttctgtgaatatttgtggacttgctcaggactctgaaaacctcaagtcaccagacatactggttcccagctgagatcaaacagtagccaacacatctgcatccttaaggaagggctttctcctgattccaggaggcctctgggctcttctggggctccacagcctctaaagtgaagtctataatggagatgtcttcccagcctggggtcacttagtcagtactactcaattctaaaatgaagaaacactaggctatatcaaaacaacaattgtccccacatcattgtgagcagatttagagacagctggactccaggagaacctagtctacatagagggacacccagccccagactacaagcttaaccacatagcttatttcccagtgaaattaccttcccaatgaaagtgggttagcaggaggtctctgcttccgatctgttatagaatgtctgctgccttctgtcagacagttgaaagggaagttgggtaccacactagccacaagaaggaagaactaggccacagtacacagtagtttgagacaggatttgaacaagcagacctggtgggaagcctggtctgctgctagcagcaagtcaggaaaacgatccaagtttcactttgcttttccataaaggagctaaatgtactctctgcttagcacgatactgtgtgatgtccctaaagtcctagctcagtggttctccaccttcctaatgctttgacccagttcctcatgttgtgatgacccccaaccacacaattattcccattgctactccataactgtaattttgctactgttatgaatcataatgatatctgatatttccgatggtttaggtgatcccaaaggggtctcaccccacaaactgagaaccattgtcttagatgcctacagtaaggcagtaatagccattaacaagaaagcccaggacacaggttttgaaacaaggggaaccagaactgtgcccaccactaaaccacgtgactgactactcctgggtctcagcttcctcatctgtaaaaagcaaatgaaaacgccagctttcactgagttctggaaggataaaacaggatactgcatccaacatgtgcacacgtgcacacatgcacacacgcgcgcgcgcacacacacacacacacacacacacacacacacacgtagcacagtcagtaagtggtgctggtggtaactgaaatgctaatactaacttagatgcttgttctggctgcagagaatcacagtagcaccaaaccatcaacccaggacagcagagggtaggggaggctgcaactgttttggtgaaagtgaaggttctagaaataacagttaggattgcctcagctcctcctgagggtcatgacagggacagagctccagggaccacagtgaatacgttgacatgacttggcaggataatgtagaaaatgagaaaggcagggacagaaatgaccaagaaaccatgaaaagatgcatttgccttataaaaaagccacaaatgctttctgcacatttgtatcgttcttttttaaattactattttatccctcctcctcttcctcttcctcttcctcctcctcctcctcctcttcctcctccttctcctccttctcctccttctcccgactttgagataggctggcctggaacacacaggtttgcctgcttctgctttctgagtgctggggttaaaggtgtgagctatcaagtcttttttatttattattatttagtgtgcatctgtacatgaatcacatgtgagttcctcaatccgtgtgtgtcagaggacaactctgtggagttggttctctccgtccacgcctacatgggtcccagggtttgaactcaggtcaggatatagcaaatgcctgtacccactgagctgtctcaccggccctactcatgtcgttctaaacactgctaatctcttttgctcttataacatgtcttacttctgatttttaaatttatttcttgatttatgtatttacttatttagttatgtgtgggtgtgcacatgcatgtgcccttgcaatgcatccagacatcaatgtcaggacaacgaccaggactcagttctcttcctctaccacgtgggtcccaggaaccaaactcagatcatcagaattggcagcaggtgcctccccctgctgagccatctgtctggcccatttatttttctcttttgttttcttaagtatgttattttagttggggtttctactgctgtgaagagacaccacaaccacaacaactcttataaaggaaaacatttcattagggctggcttacagttttagaggcttagtccatcgttgtcgtggtgggaacatggtgacatggtgctggagaagttgagtcctacatttggatcttcggggagcagaaggagactctgcctggccgaccagacttgagcttaaagacctcagatcctgcctccacagcaatgcacttcctcgaggccacacctcctaacagcgccattccctatgggccaggcattgaaacatgtatcgtgagggcccttcctattcagcaccacagatatcaatcagcctgcattcatgctcattatgaggcaagcttgtcatgtcctttcctgatacaccagcttcactaactggttacgggcccttcttccattctctgagattgtataagatcaaaactaatgctccttaaatagctcctgttagtgcttttttctaaaggaaatatttcatctacagatgtaactgctttgataatcataggatagtatgacattattctttctttgtaaatcagtgcttattgttatttttctaggaatttgtggtaacttcctatgaaatttttgcttttgctaaaaagaaaaaatctatcttgttagatgtaggtacgtaaacattttgcgtgtttatataaaatatacttattctttcacttttaacattcctgtgctctgataccttaaaatcatctaaaactctaaaaagtattcaatcttggagatggagagatgatttaccatctgaccgccctagcagaggactggcgttcgattcccagcactggtatgggtgtggtggatctcaactgtccgtagctcgagtcccgagggatcagtgccatcttccagcctcaggcaggcacattagtgaatacagtgatacactgcagccagaacatcgaaacacattaaaataaatagattttttaaaatgttaattcaatctctagccagctttggcgagtaagaatcttaaatcttaggactcagaggaggagggaggcaggggaatcaggaagatctggaggtcaagccttgtccacaaagtgagttcaagtccagcctgcagtatgagagcagtttcccaaaaagaagaaaaatgcactccaatttttctttattatttacgtaattttaaacatttatttgtttattgtgtatacatgtgtgagggcaataagggatggacagggtaatatgcttaacatacaatatatgccagtaaaaaataattgaggacaatcttctttttattattgagaattttatacatgtatataatggggttttgttttgttttaagatagggtctcattatgtagcattgactggatggaactcattatatagaccaggatgacctcaaaatcacagagatcttcctctgcctcacaagtgctgggatcaaagacatgtaccaccacctccagccattaagatgttttgatcaaattcacaccccagctgggcgtggtggcgtatgcctttaatcccagcacttgggaggcagaggcaggcagatttctgagttcgaggccagcctggtctacagagtgagttccaggacagccaggactacacagagaaaccttgtctccaaaaaaaaaaaaaaaaaaaaaattcacacctcgtccctgctgctactttcccttcccctcccacgccctattcatgtttgttttgtgtgagtttgtctgcatgaatgcgcaccacatgcacacagtgccccgagagacttgaggagtgtgcgccacagctgttgccaccatgtgagtgctgagaactgaacccagatccgctgcaagaacatggtgcttaaccttctctccagacgtccttttcttttctaaagcccgctggttgtacttagtgccatctgtgtgtgcatggatctaggaccatctactggagtatgggcagcttttgggggccacatcccagaagaaaacttattcttcttcccccagcagccattattaattattattaattcttctcagtcaggaaagcatcatgaactcctccctgtgagctcccaagccagatgagaagtggtagagcattgcactacccagacaactctcagaaagcacagcgggagtggaactgatccctactcaaatcaggcttgggcctactctgttccatagagacaaagaccacgtcaggatacagctacttaggctgctgtttccctcatcagaaaactggctcctcagaaatcacatgatcagcagttgctaatctggctgaagcactttttcctttgtcctttttccttccctccccccatgactcaagccagccccaaacaagctgttctctgaaacaatactcacagccctccgcatgttctccagctccccccacccccaaccctgagactgctgtgggcaatggccaattagggccaggggacccacttctcctcaatccatgcagaaatttttggcttgtttgatcatttgcaggtatgcaatcatattcactgtttgtatttgtaaacatacagtcatatctgccaaatactgtttgtctacagacacccacctctgctggctgttagagtctttttacttcctcttctgcaaggtctctgagtcttgagacgaggggctatgatacagatgttccatttagagctaagcacaccacggtcttactctctgaacactgactagctgtagatctctatattggctgttgtctaccgcacaaggaagcgtctctgataaggttgagagataaactagtctatgggtataaagacaaggacatagaggacaatgtaatactacatttagcaaaataatagtattagggtctccactaggtactatggcctaaccagtcgtgggatttggctcagttaacaataccagatatgagttccatcatgtggactggattttaaatcaaaccagaaagtggttggttactcccataacatttgtgccactatcacatgagtgggaacattttgccaagatagtcattgtagttcatggagttcatggctagataaaagtgcttaatacctttctccccgagtagaataagtagaatatggcactataaaaatctatccagtagaaatgaagctttttggttactaccagcttgagttacattctgtgactgaagtgtgtggtgtgttcagggactaggtcttactatcaagctctagcaagagcgagagcaagagccctagcctgtaatgtttagaggaatccatggtccacaataaccagcaatatgaaaagagataacccatacctagcattggtccttttatttgatagcctacagtgcctggggaggcatgctcctctattatagggttaatttaatttaaactctcttttatatgtatatattttaggaagttttatacaatagtaggtttctatgaggcatttccagtggtctttagtgttagtcatccttcctcatacgcccctttattctaccctctcatatcccagcccatttaagaaaacacactttttagttaagaatcattttcggctactacagaatcctggttatagaaatgattcaaattttagtggaacgaagggtctcctgataaaatgatttagagtactggacctgggaaacagctcagtaggaaaagtggtataaatcatgcgagcccaagttctgccccctggacccaagtttttaaaaagccaggcgtgacagaatgtattcctgtgatccctacgctagatacacagagacaggtgcattcacgaggttcactggccaaccatcctaggctaatcagtggactccaggttcagtgagaggaccctgttaccagtggtcagcgtctgagctgggtgtttccagggtcttgacctcctacccaaagaattgaaaataagggcacacatagattcacaaaagaagcaagataattaaattgtaagggaagcagtaaagcagattatagggaggtataatgtcaagatggacggcaggccatgtgagtgctcatggacctaactcatgtttggaattttcttttatgatatgggagagaaggaggaattgattactaagggtatagtttgggcaatttattttgattgatagactaggccatacatttattttccctactgtgcctgttccttcccacaatgcgtctgattttggtctctctctctcacatacacacttatatacaaacacacacatacacactcaaataatacattcacattcacacacacacacaccgagagagagagagagagagagagagagagagagagagagagagaataatatacacagagaggataataaataaataaataaatgcagaagccagcccatacctcaacttggggatatttgaatcctgagattaggccacaaaaacttggatttttatttgtttgtttgttaaattttggtttgcttttgcttgtttgttttgcttttgttacagtcttcctctataattaatggtggcttaggactctccacgatggccaggctggcctataactcaaagaaatcctcctgcctcaacttctcaagtgctgggattgtaggtctgtaccatcacacccactggaaagctgcatttcacatggctgtagcatttaatgtggatgccagaagttctgactacgctttaagtttcacggagtagatgttggcagaggatgtcagtgaactggtggggactggacagcctagcatgcagcctgccagtctcaccttcccggtgtcttcagttctctctgtaaacgtaatgacagtgtggggaagccacgcactcttaggatagtaccccagcacacacaggctgggatgcaactcagtagggaaagagctggcttagtatgtgtgaggtcctgggtttgatcccaagcaccacccaaccagatgcactagtccatgcctgtgctctcaggactagggaggtggagacaggaggatgaaaagttcaaggtcatcttgagctacatagtaagtttgaggccagcccaggctacctgagaacccctgtctcacaatcaaagtgaaacataaaacacaaacaacagaaagcgctccaacccacatttacctgctgctcccaactcacacatttcctacccagggatgctaatcgtaaagcattaggtgtttgcattcgccctggagtcaactgtaatagggatgcttgccctctgcatcctcatagatctgagcccctaaggtctgcagggggtggggcaggaacatcgggagaaggaggcttcattaccctcagacaaaggcttgcagcagattcctcctatgcattcagcagcctgtataggattagtgtatacagcatgtttagaaggccccatgctatatcccattgaccgccagtgtcaaaggcatgaatgccttttgctgaggcaaggggagctctctgaaactatattttctctttttaaagcaagagtcccagcaaggctgttggaggtgccctaaaggtccacaaggcattgttatctgtcccctgagccatctctggctggctcgctccccctccctgactcctttccgacttgcctgtgttgcccctagaccatgggctcaggggcctctggacaaagctgctgactctgaccacagcctttctctggaccgtgttcatttgcatcaagctgctgagggtggaggagctatataggctctgggccattcaccagaggattaggcctgggctgagggtgcagctggagaggccgcagggaaggccgggttccgctgttggtcatcatgccctacctgctgccgggattcttctgtgacagggtgatccgagaaagggacaggagaaatggagagggcactgtctcacaacctctcaagtttgaggggcaggattttgtcgttctcaaacaacagtgtctggctcagaagtgcctctttgaagaccgtgtcttcccagcaggtgtgcaggcccttggctcccatgagctgagccagaaagccaagatgaaggccatcacttggaagaggccaaaggtagggataaagggggcatggaatctgtagacagatgttggggaactgggagtaaagtggtcagagcgtggggctgtggattcaccccaactctgagcaactagcttattaatgtcccccttacccataagccccagctccaaatacctaacttcttatgctagaagctaaggatgttgttttctattcctgaactgcgtggcgagggttaggatggcctttagatccctgagattatatttaagagtgccaatgcattgatttaaattcccataagattcgtaggtagaatcagctcggttcttcccgagaccaggagtttggggagggaagagtggagtggtgaagagaacagaggcttgaagtttcaggtccacagaagggatggacagtgaattgacaggaggctggaggcgagccttagccttctggacgaagctggttcattagcatggtgtcaagtctgcttaaacagaaaagtccccgctgaccagccagacagttgatccaggctgccctatcaaggggcatcctaggagccccagcatggcagcaggaggacgtcaaagcccttcttcctgcaaggcggtgcctatagctgcaagggagcctgctatatttagagctggcaaggagtgtgcgtgtgcgtgtgcgtgagtgtgtgtgtgtgtgtgtgtgtgtgtgtgtgtgtgtgtgtgtgaaaacccagaacatctgcaaggcacattctttactgtatatgcaatttttaattagttaaattactttttaaatttttaattaaattttaaattaattttacaattacaggattacataaaaactgtaaagatagcgctgggctcccatgtgtagtccctcttacctgtgcttttcactctctgcagtttttgctgacctgtagtcaactggggtgcgaaaatactaaatgggaaattcttgaaatacttcagaagttttaaataacctccactattgtacattgttttaattattctattttattgtagtattagtcatctctcattgatttatgagttaaactattggtgtgtgtataggaaggagtctaatgatctatatgggcattggtcataaccacagtttcagattcctgggggtcttgggatataccccacaagtaaggagaccgctacctttagtccagttttccttgttatatcatgacctgtacctcggcgatcagtaagattgatttgttattgaaaacaaagtccacacgttatttccttagggttcttcctctgtctttttatgttccaagactcctcccttgtattcacttatgtgtcttgtgtccttagcctcccctctcaagttttcctggtttccaatgagctgtgcactttcacagagcactaatcaggtattttgtaaaacacctctcatggcatctctctcgaattttcttccgagtaggctgaacttatggatttgtttgggtttttttatatataattttttatttattttaaagtagctgagtatttaagtatgtgtataccacatgcagtgctcatggtggccactggatggcactggagcccttataactgaccattacagaccaactgagctacagaccattggaaggcaggtgctcaacaaaggctgagccatctctccagccctgggcttatgtgctttgaggaggctgatcgcagaggtcaggcacagactgtaaaatgcctcctcactgctcacattgactactttgatatgttgcactgctttgggtgcagaaagtcttataattctgcatgagaggtctgtgtactctgtgtgtctctgtgtagccctttataccaatactgacttatgcctattcattgccaggagagctatggtatgatatggatgtgcctttctatgtagtaggatggagacaaccttgagtgtgagggcagagctcagctcacttgggagaattgtgtagttgtgaggaaggagaaagacagtgagacagaaagacagaggacatgtctaattagggtttctattgctgtgatgaaacaccatgaccaagacaacttgggcagcaaagtgtttatttcaggtcacacttccaggtaaggaacggcccatcactgagggaactcagggcagaaactcaaacagagcaggaacctggaggcaggagatgatgcagaggccatggaggggtgctgcttactggcttgctcttcatggcttgctcagccagctttcttatagaacccagtatcatcagcccagcgatggccccacccacgatggtctgggccctcccccctcaatcactaatttagaaaatgccctacaatcacatcttatggaggcatttttctcagttgaggctccctcctttcagataactttactgtgtcaagctgacataaaactagctagcacagaacacttcacaggaataacaaggtttcttgaagctgtgaagaacaacattaaaaatcaaaacactgaatctttaatgagtttaaaagttttttaaaaaattgccgggtgtggtggtgcacgcctcctttaatcccagcactcgggaggcagaggcaggcggatttctgagttcaaggccagcctggtctacagagtgagttccaggacagccagggctacacagagaaaccctgtctcgaaaaacaaacaaacaaaacaaaaacaaaaaaatttaaaacgaagcagacactgaatgaaatcaagagtgctgtctgttggctgctgatgggcgtgacttagcccatgggcttggaaactgcagctgccaccattgctgctgtgggtgtgtttgccccttccaactggggaggggggaagaggtgaggctctcgtggggtttacagtctgcttccgcaactgccaaggcctgtgtccggatttctaaaaagggaagattgttctcctcctcctcctcctcctcctcctcctcctcctcctcctcctcctgctcctcctcctcctcctcctcctgctcctcctcctcctcctcttccatcctgtgccccagtttacagaaaacaaagccataagttctgcagcctcttgcccgtcccccctctcaaagaacattctacccagtacgtggtatggaaagaagaggggacagctttgaggctcttacctcctctgaccctcagatgtttattgttcattttgagacagagtctcccaatgaagcccgggctgaccttgaactagccatgctcctgcctcagctcctccagtgctgggattacagctgtgcaccaatacaccaacttctgagcttcagctttctggtttcaaaagaagagcgctggctctcctcttggatggctgtggaggccggggagggggacagtgagtgtaaatctttggcccccagtgcagtaggccatcagctcagccaggactcctacaatggtgttacaatggttgtttctgttttgatttcacaggaaatttgtgagaatccccggtttatcattggtggagccaacaggactgacatctgccaaggagatctaggtaggaaaaatcctctatgtcctaccaggtgacccagggttagttaacctgagagatctctattcaggatgggaagggataaattctggtttctcttttagtgcatctcctaatattttaaaataatttttatttatttatttagtgagtgagtgtgtctgtctgtctgtctgtctgtgtgcacataccatggcctatacctgtgaaggtcaaaggacaccttgtgggggccaggtttttccttcttccgtgtggcatctggaggtcagtctcaggtcctcagatcaggcatctccacactaaaggaagtcttttggcagaggcggcgtctgccatgtagaggagaggacaaggttctgtcaccttgtcgtctggactgttctgagtcccttcctctcctccaccttttgttctggaagcatcctccctaggcagactgtaggagagctctgtgaagagaaactggagagaacattaaaggactatttaaaacttgacccaaaaaaagcaggaacaactgaaaaatacctatcactttgttccagaaagggaggctgagaacacctcaggtacagtggcttctggcatctgagggacactgcctggagggtgtggacaaattaatccctgagccctcaaaccaatacactgcttctgagctgagtcaaggcagggagatagggggtttgggtggttacaagcctctcttcccaccagcagtgtggcagctagcctgtgcccccagtagagctatggaattgtcagggtaccaggcatgtccctttggcagccttagtgacctgtgtagaatggttctgtggcagctagctgcccaacacccctgccttctgacacacgtctgcttagctgatggttctctaatactgttttccgtgatgccagaaggggaaatgggctcaccctacaaagacagaggacagccataattaccactgttgtctctactgagaccatagaaggaagtgacttatttatttatttatttacaatgtaaaaagcaagaggaatttattgttccaacatgttgcggttgccctgcacatggagagagagaatgaccaggaagtgttttattaaaggaggaatccattgcctaacttaggaaagcctgtcttgagagaatgagttacccggtcctcgtgctgctcgtgccgttgaggagctcctgtccctgacagtctccttgcttgagctcacacctacttgagggcaggcaggaagcagggcagtgggcatcccgagtcattagctatggggagcttcttactggggctggcagggctcttctgagaggggaagcccccatctgactacctgggtctgtttttgcttgcaggggactgctggtttcttgcagccatcgcctgcctgaccctgaatgagcggctgcttttccgagttataccccatgatcaaagtttcactgaaaactacgcagggatcttccacttccaggtgaggaaatgggggtgtggttgagacagcagccatacctcagttaaagataacctcagagaaggcaccaaaatgttccgtgctgtagtaaggaggtgaccttctcttgtctgtagtctgtctggtgtctgtgtctctctctgtgtgtgtgtgtgtgtgtgtgtgtgtgtgtgtgtgtgtgtgtgtgtgtgtgacccatctgtccttgttgcctgtgtatctaatgaatgtatgtgtttgctgtcatgtctgtgttttttctcatctgtgcctgtgtcatatgtctgttgcctagttgtatgtgtgtgtgtgtgtctgtctgtctgtctgtctgtctgaatctgtgcctaacagagggctttggtttgtattcattgaacagtacagaaattctttgcagaaatcttgaacagatttttacaaggaaagaaatcactttattgacctcaaatcacccagagaatcaaaagcacaaacccacagacatattatcaacttacatccacgtgtggcttccaaaatggggtagttgttgcgtaccgttgcttccaacctcttggctgttttcaggaaattattaccacagcgtgcataatcttaggtcatggtaatagtaaagtacataacttgagattaaagctgtgtgtttagcttaggttataaaggctgattacttgggaaatcgaaagcataatgaggttggctgctctgttctctgaaaggcccattaaacttccccaggctgagtgcagagaggccagcaggctaagtacatagccctaaatgatctcaaggcatattattaacttggctgcaaggtcccggaaacgttcagtctcttctgagtcaagagatattttcagacaagtatagcaccacccagccacagtctggccaccctgggtctcccggccttgacttcccagctggctcaggaaaggttctacacacctacctgtgggcaatgacccacttacagctacaagcttgcaaagaagcaggtgtttatctgtgccctccaacttacatactttccactgtgccccagaaagggggagagaaagtcatccaggctgctggcttgagtgcctattcttgtcctccagctggctggctgctatgttattgcctgaagcccccccccctcaaacaggttcttaagatgctcagcctgaaagtgttggattttttttttctgtgatccctggctcatgaccctgtacccttgacagaggagtctatttaccttcctactgctatcacttacctttctaggtcttaccagaaaattctggtcttctgggcatttagtggctggtttgacccagccctgaaagtattggttctagaattaatttatccattagaatttttctgggcactgatcatcttgatgcagagacaaattctgggttggatcagaacagatctgagacctgcatgtggggctctggaactttctgtggttctgagcagcgtgcctctggccctgaaacttcctagtgtcgagaatgactcaaactgatcagctgacatcccactcctatggtacctcagcatatccagtgcactccccttttccattttagccatcaatcaataaataaatcagttgggtgtggtggcacagacctataatgccagcactcaggaggcagagacaggaagatcaagataagttcgaggctagcctgggtacctagtaaaaacaagttgtttatctgtgacccctgacttctcatccttgacgatgtgccatgggaagggagggaggaaggcatactgcctgctgacctgtgtgcctcaaacacaatagaaaggaagggaggaggggaggggaggaaagaagaaaatgggataaagagaaatactttagttagccctacagctttccttaatcatgagttctcaatcctgggcgggcactggaatcacttgggatacacgaatgtatttaaactaggtctgtcccagacctatgggcaagggagaaaagtctcaaaccctaagatattcagaggacctactatgtctagaactgggctgggcttttgacagacactaagggatatgcagtttcctgttttgcagaagttcatggcctagttagagatagatttacacactcaaaagccagactagaatgaacaagcttcaggatgcaaagcccaggaaatggtagggttttggctcagagtcctataggatgtgggatcaggtaagggagtgcacaggggtgactgggcttctttcttcttgcagttctggcgctatggagactgggtagatgtggtcattgacgactgtctgccaacatacaacaatcagctggttttcaccaaatccaaccaccgcaatgagttctggagtgctctccttgagaaggcttatgccaagtaaggcaacatttaggatgggaaacatttaggactacaggggtgggggatatggatttcaagattcagccaaagtctccttcacatggggctaggcatgtgaatgcatgctcatgtggattgtgggagtccagtaaagagcccgagtcagcttgagatcaggactgggcagtaggagccctatctgaaaaacataaaagcaaaaagatataaagatgtgtgcagtaagtcttcagcttcctacagtcagctctttgatcctgccttcatcatggtgtttcaagtcttcttcatcagctcattgcttctctggagttcagctcctaggatcttacttgcttaaaaccatatacttgtagcacattcataagaccaggttgtctcttaccttagccctccacctccgccactagatttagcagatagtggaagagggctgtgggaaatggggaccgtcatgacaatgcttctggccctctctggcctagagttaactcagcttgtcatacttcctggaactcctagactccagactgccttttgtcctctctccaaggctccatggctcctatgaagctctgaaaggtgggaataccacagaagccatggaggacttcacaggaggggtgacagagttttttgagatcaaggatgctccgagtgacatgtacaagattatgaggaaagctatcgagagaggctccctcatgggctgctccattgacgtaagtgtgattccagggaggcccttcactcagctaacaacaacacacagcttcctcctgagatggggtggatgctgtaaaagcaagagccacattggtgagatggctcgctggtaatgacacttgttactaaacctgaggacctaagtttgatctggaatccacatggtagaaagacaatggactcccgaaacctgttgtctgacttctgcatgcacttcctccccacactaaataaacaaaaatttttaaaagaaactaccagtagttccagcaattgaaaagttgaatccaacctgatttgattagtgaggtcctgcttctaaaaacaaagaacatggatgatggttaactttaaaattgacagtttttcggtgggaatgtgtccctgaaggtggggtgctttccaagaaaaagaatgagccatgacactccaaacattagaattctggtagcaatagaccagaggccccactgattggcctctcttgtccagactgctcagcacaagcgtgccccacaagccaaccacatcgctggctatggatctttgcccttagaaagttctgtagtcgctgagttttgacatcacctcagtccttggactctgtgctgcagtctggatgatctctctgaccctctctcagggtaccaagatcatatgaactgagacaggaacaaggagaggacccaccatggcctggtttccccaagagctgctttgggaatagtagtctggggtggggcaagccacctcctgcctctttctctcccagcactccctgtgggtaggaaggcttggttgtcaccacatgggagaactctcctgtcctgtcattgtcttgtatcttttctgcatttacgctctgttgatgtctttctcctcttttgctttgcctgtcgctcccccttcccttcccgccctcccttgtttgcactctttctttcctgggttctctactttccttcccattgggcctcaggatggcacgaacatgacttatggaacctctccttctggtctgaacatgggggagttgattgcacggatggtgagaaatatggataactcgctgctcagagactcagacctggaccccaggggctcagatgacagaccgtcacgggtgtgtacatctccaaatgtcgagactgactagcgcccccagatctgcatggcccatccttctgaagtcagtgccaacagaatagattccctccattttggtaactctcctctaaggaacacagggcttcagtgctatgtagtgcttgcatcctcctgtttgaacggcccatgtgcttcctgaaggactgagccagacagaaaagcacagaggaaaagttagtctatattttctcatctaaagataactgcccagggtttggtatatggtccttctgggcacaagcttaaactatatattttttaaaattgtatcttagtatatgttacatctttacagcctgttttttgttgttgttgagttttttggtcctcccccccccccaccagcaagacagggtttttctgtgtagccctggctgtcctggaacttactctgtagaccaggctgtcctcgaactcagaaattcgcctgcctctgcctcccaagtgctgggattaaaggtgtgtgccaccactgcccggcaaggttcatatttttaaaatgcaactaagtattctttttatatcattttagtagctgtacaatattctagtgtacaaatatattataatttacttactaacactctattattgaagagttagagggtttttttaaagtctgtttttcaaaagtgctgtaataagcataaagacagttttgtatacacccataatggtttctataggagagtctcaagaagaaaactgctggtcaaaatgataggccgtgtagcgcgccaaaagccatctcaggcagcctctgagctatagctgagcggattagtgagtgccctgccctaatctgagtcagatgaatgaaatcgaagacactaagatgaaaaaagttccagactcacctacacctgtcaccctgaaggcggcttcaggaattcacctccatgtggctcccagaagcttccccaaagctgttgttactgggccagcggatgctgagatgcctgtcgagagctcagagggcgttggtggtctatctacccttgtgccgaggaaccctcaagaactagaacttgaacatctttgcccctgaattcaggcatagtcctgtcacagcagggaaccgccccgtatgtattgccagcattgggagtcttgacttccatgttctagtaagtgtgagagaggtgaagtgacagcaagagaactctctttctcaaatccttctttggggaatagtcaactctagatgggctcagaggaaggcacagaccccagatgtccccctctcctctgctgaggaagtctagaatggaggggaaattcttttacttacctcctattccaggactcaccttgatctctggccatcagggcagcaagatcagctaggaggagtagggcagcatcttcctgttgctcttgaaaaaccagcgagggtctgaccctggccataaggcataaggtcaaggaagagagtcatctttagtctgagataactcagaaaaggaccagcatgaacccttgtctccaatcagcagaacttctgccctccttcaccccaactcctgacaagctgtctggttatttctctacagacaattgttccagtgcagtatgaaacaagaatggcctgtgggttggtgaaagggcatgcctattcagtcactgggctggaggaggtgagtctgttggagcttggtggggcctggaaacccctgagctgacctgctaatgtcaggacttaactttctgtgcctctctcatgacccagagaacttgccagtcaggtccaagctgggttcctgagggcagaagggacctatgggactgacttgaggtagacctggccgctaatcttctgggttcagctgtgtgtctagtgaatgtacacacttatgtatttgtgcctgtggaggcaaggggtcagtattgggtgttccttggtgattcatcttattttgagacggtgcctctcactgagcctggaccttaccaattggttaggttggttggtcaatgagctccagggatccgccctccctgtgctggggttacagatatactttgctgtggcatcttttttacatgggtgttggggatccaaacttgggtctctattcttgaccagcaggtatttcatcagctgagtggttccctggcccttccctttgcttttaatgctgaagactgggatttttctagttgtttccaaggaacaactagaaacacagcttttggcctacaaagttgagcttgtgtactgccttgtttcctgttcactcgtgtccattttgcttcctgtggggtttctgaagtgtcctgggtcatttcccaagcaggatcattgtgaggctgtagcatgcagttggacaggttatgtcctgaaaaaaaaagtggcctaagaggctgagtctgcatcccccagggcacatgactggagaatggggcatctttttctaactgatctgttaaggggaggcatctgtctgtagtttatctatgaggggcctgctttaaatcacccacagaaaggtgtagtgaagggtctcagtagaagcacttactgcgcaagtctgaagactcaagtccaaacctgggacccagagcagaaggagaggactgactccaggaagttgtcctctattgtgcacatgtgcactgagacacatgcacacccaccctcacatcacacacatacacacatgtgcacacacacatgcacacacacacacacactgaataaaaataagggcaggagaggtggctctgcagttaaaagcatttattgttcttccagaggacctgggtttgggtcccagcaccatcaggtaactctagttccaggagctctgagaacctctggctgcctgctcgggcacttgcaggcatatggtgcacataaactcacatgggcagacatacatccacataagtaaaaagaataaagcctaacaaaatacttaatatgtttctaaagcctcatcataaatgtttatccccttccaccagtacataccatggccagatgcctcaccctgtcagcaagcttctggtttgtccacctggccagggccaggcaggcagcctttatactggattctcacaacttctacccagccctcataccaaatcagatgccccatctttccaaacttgcttgagcttccaagttgtgtctctttgcctcaggccctgttcaaaggtgagaaggtgaagttggtgcggctgcggaacccctggggccaggtggagtggaacggctcttggagtgatgggtgaggcagtccacggtcaacagagcttggggccacactggggtgggtaccaaaccatgagagtactgttgatactggtggaagatcactctcaaaccagtgacctacagagaaggacactggtgtgggttccccaaaagggcaggaggtgaaggcaggggagccagggactcagtgaacatgatggtgcttatattcttgaggaaaggttctagaagcctccatcagagttctggactaaacaggcaattcccttcttagtatctatcccctttacctcacctacaaaatggaagcagtacctacagtgtccaaatgactcttatgaggggtaaaggtgacagctggggaaagctcttaactgagaccaaaagagcctctcttttggagagaaagaaagaaagaaagaaagaaagaaagaaagaaagaaagaaagaaagaaagaaagaaagaaagaaagaaagaaagagagagagagagagagaaagaaagaaagaaagaaagaaagaaagaaagaaagaaagaaagaaagaaagaaggaaagagggagggaaagaaagagagaaagagagaaagaaagaaagagagagagaaagaaagaaagaaagaaagaaagaaagaaagaaagaaagaaagaaagaaagaaggaaagagggagggaaagaaagagagaaagagagaaagaaagaaagagagaaagaaagaaagaaagaaagaaaggaaggaaggaaggaaggaaggaaggaaggaaggaagaaagaaagaaagaaagaaagaaagaaagaaagaaagaaagaaagaaagaaagaaagaaaaaaagaaagaaagaaagggaaggaaggaaggaaggaaggaaggaaggaaggaaggaaggaaggaaggaaggaagagtgcttagctgttttcctgttacatgatcttttcttagcaactccgtgggcagcaagctaacagtttcgacgagttagttcatgaagtcctcatagctcctagacagatgatgtagtcggttcccttctgcagcgaggaagtcgaagctgacaggtcaagtgacatgtctaaggatatacagctgtctgatgcagattcaagctcttaatgaggaggctagaagattcagccagagacagccaagcccttgtgaacccactcgaggggactggtcttagaactttcttaattcccccattttcccaccagttggaaggactggagctttgtagacaaagatgagaaggcccgtctacagcaccaggttactgaggatggagagttctggtaagttcatgacccaggcagggagccaccagggtgcatctttggcatatctgggccctgttttcctatgggtgctcaaggcccagcaaaggtgaggttctgaaaagagagtgccagtatggcagtgactgagctcagaccctggccatcccatggcagctctcgttccagaagctacccttgcgcactggccctgatgtggctctcttctgaaccaaggggcttttcttacctgatatccagtgccagccttattctgatcatgtcagccttctgagtctcagctgacaatagtgtctcctatgacgcctttggttccctcaggacccagaggcatttatctatgatgtcactggccttaaaactatcttttcccaagtgacatctctttttccatgctgaaaatgtgcctggcaataatgaaacatacagtgagtaacattttggagaactgatttccagcacactcttttcaaagacaaatgaggagtcatgaacataagacatggtgatagtttggggtagataggtcagaaatgacagtgccaggcacacacctgtaagtccatcatttgggagacagaggaaggagggtcaaaaggtcaaggccaaccaagaccacaggatgaaatcctgtctctcaaaagcaattgaacaagccaagtgacatcagggtcttctgggggactttgctgtgctgtattagcccttgaatcttcccttcctcagcctgtgtgagtgtctcctctctcaacctctcaggatgtcatatgatgacttcgtctaccatttcacgaagctggagatctgcaacctcacagctgacgccctggagtccgataagcttcagacctggacggtgtctgtaaacgagggccgctgggtgaggggctgttctgctggaggctgccgaaacttcccaggtgggagagcctctggatgggggaaggtctaggcagaactcattccaggcagaataatattagctatcacttatggagcctgtcctaaattcaggatgcactgtcttattaaagcctcctcagttaaggaaggaaacatgccaagggacctccgacagggagcttgtagcaacttgagcttgaagcaggaaactgtcagagcgccacgccaatcctgctggaggtgcatttgaggacctggtggccatggcacttgcccatagcagagatgctgaatgcatggccaaggcagtggcctcagagatggggcagcagagagaatgcagatttagggaaggaataatgaacgataccaagggtcctgctgtttttatgtggctggttgtggagttacagtaagaggagtttagtttgtgacatgaaaagtttgaggtacatattgactaccatggtaaagatttgtagtagagaactgaagtcatctacattttagagtagacttggttatatgataaaaattcaatgaagatgacagaaagatggttcggcctataaagaattttccgggtaaactgagtctggatcctcaaacccccacaaaagtcaggtgcactaagttgtgtgtccaagaccccagggatgggaaatggagacaggagaatcccagaagctcaggggccagttagcccagcatacacatcagcagacaacagaggctcccaagaaggtggaaggaggaggactgatggtcaacattgccctttgacctccatgcacatgccatgccatgccgtgcatacatctgctttcgcaaacacacacacacacacacacacacacacacacacacacaccatatacacacaaaactcagtattttatgatgaaaaaaaaaaaaaccaaacgagtaaatagatgagaagaaaagggtggtagagagtgggaagccagataggaaactgagcctagacatgtacctcaggtgtcattggaatgtagggctcagttagaaagtagaggtcacacaaagctatggtggtgcactcctttaatcccagcacatggaaggcagagacaggcagatctctgtttgaagccagcctgatcttcagagagagtttaggacagccagggctacacagagaaactctgtctctggaaaaaagaaaagagacactaacaccacacatacacacacacacacacacacacacacacacacacacacacaccaccaccaccaccaccacaccacatacaccacatacaccacaccacacacacacacacacacacacacacacacacacacacacacacacacacatcacatcacagtggagagggtcagatgcctagggaaacagcataggtgaaatcagaggagaaacaaagcctcattcacttgagaccagtgacacttgaggagtaggctaaaaagttaaacagctgtcatgtgtcaaagacaatgaacgttcagaaaggacatgacagcattgcacatccagaccgtgcaatgccagaagcgctggtttagctttctacagctgtggtgaaataccaagaccaaaacaacttggggagaaaagggtttatttggcatcactgttcatcagaggaggaattcaggaactcaaacaggcaggaacctggaagcagacgctgatgcagagaccatggaggggtgctgtttaccagcttgcttgatttttttatagaacacaggaccacaggttgtctgcttacagcccaatcttagggagacattttatcagtggctgagaggaggagattccctcttttctgatgactctaacttgtatcaagttgatataaaattagccatcataagggtagggggtctcactcaccaataattacacaaacgcttggatcatgctttccataaataactgtgcatcttacagttgtccggaatgggaagcaggcctcttaaactttttttacatatatcttcttttcaatgaggaatttttacacaaccccgtgtataggtgtataaaataggtatgcaaatcagacacttactgataaatcattaagacatttacttgggagtctgggagtgtaacacagttgatagtttgcttgtctagcatgcatgaagtcacaccctcacacacatacacacagagaataaagcatgtatggtggcacatgcttctaatcccagcacgaaggagtcaggagaattgtcacaaaaaatgaaaagaaaataaatttattttattttaaaacagttcatggtgcacatgtaagtttgccatttattttatgttaatgagataggtgcacttgttcgcttttgctcaaaaaaattaaatcttgtggaatattagataccgtaggacacagagcatcttctttttggaattgatggatattttgatgtcataattgtcaatcttgagaatcccatttatataaataagtagtacgaatcggcagcagttcttagggccatttcagaaatttctgggaattctgtcctaattctaagtcaaaatccatttaatgggaactggcgagatgactcaatggtaaagagctcttactgctcttgcagaagacctcggttcggttcccagcaatcacatggtggccctcttctgatctccatgggcaccagacatgcatgcggtacacagacataggcaaaccatccatacacataattcaaaaccttaacgttacccatcatagggaacaaaggagaccggatctctgtcctactttccttcctgccctttctcatagaggcgtatctgaacacgcatttcctttcttgggtcacagacactttctggaccaacccgcagtaccgtctcaagcttctggaggaagacgatgaccctgaggactctgaggtaatctgcagcttcctcgtggctctgatgcagaagaaccggcgcaaggaccggaagctgggggccaacctcttcactattggcttcgccatctacgaggtatgagcgccccggctccagtacgggaaacacctttccagcggggagaacttgggagtttccaacagggtcttctggtttttcgtgtccccagtgaccaacagggctcctgctctcaggctcacacatgtctgtcaccagtgtaaataccacagttgcagaaggcaggacggagctggccgggtggtagcaggagttagagcctacagtcactctcatgtgtctggcattcccttctgcagactctgtaccctcatttacatcagagatgttctttttctatttcgcttccaggttcccaaagaggtatagcagtggcagcgggcagccgtggtgtgcagcattccttgggggtcctaagtctgtctgtggccccctaagaagattcctggtagggttggtgatcgaggctgataggagggtaccacgcccagaatgcacgttttctccctcccaccaggacaccctgcacatcaaacacacaaaccttcccacacttattctttcatattcatgcaagtatacactctcagaggtgcgtttgaaggaggctgcctcctagaataggagaatcgccttcttcagagcccagagtccttccccatctttggggcacttgtgagcttgagggatttggaattggggatcatcagttctggtgagtggccctgccaagggaaaaacatagacctgacctttatttctgaatcctgcccccccaccccctccccccaaaaaggaagaggaggaagtgagctacagaatttctgtgatgttcagcaatttgaggccttaggaactggaagtaggtaggtaaggactgtgaaacgaatgaccaaagagtcagggcccttggaggggactatgagcaggtccacacaaccttcccaaggggctcagttcttttgggttcttgtccccagatgcacgggaataagcaacacctgcagaaggacttcttcttgtacaacgcctccaaggccagaagcaaaacctacatcaacatgcgggaggtgtcccagcgcttccgcctgccacccagcgagtatgtcatcgtaccctccacctacgagccccaccaggagggggaattcatcctccgagtcttctccgaaaagaggaatctctctgagtgagtggcagcctggctttccccacctaccgctaagaggtcactcatgtggcccatcctatagccaggaggcagctggaccaatttccttcaggtcttttgggacacagccacaggcttttgccattctgtactctaggacctttactcaagttccttcagtcctttctttgattgctttttacagcactggagtgcaatcccggggcgccacaggtttaactaaacctcccaggctagccttgaactcactcagattggcctcgaacctttgatcctcctaccccagcctccagaggagttgcaagtatagccctgtggcactagagcagctcacaaatactcactccattcatcctgcagatatgttttgagcagccattctaccccaggactcttccaggcactggggatcatgaagtaaacaggatcacagaaacagaaacagaaggggctcggtgacttcatgaaggttaacactcaagggacaagggacgaagaaacacacattgctttatagacacattagatttatagaaatgataagcaagtgagtgtagttggaatgcacgggaagccagccaggcttcatttcagcagatgtactgacagaccctgcctctctgagttctgtccctggaagaggtgctaggcctgttgctaggtagctgggccatgctttgagggagctctgggcaacaccaaggagtagctgtgtgcctgggcttttttgggaatggcatctacatgaggctggctcctggggtgggtgtttagtcccaggctcttttagatttaatctcaaatctttgaccacaaagaagccatgtgacctggacaaatttgtgggggtgactcctgtttgttccgggggatattgagataccctttgtgtttttcgcagggaagctgaaaatacaatctctgtggatcggccagtggtaagtggttaggctcttaactatgagaagatatgggggtcacattccagcccaggcaggggcagtcaagtgtgcatggcagaaggaatgatgcttctgttatagaaccattttgctggtcagaagcaggagtgtggaagaggagaaaatattttatcaattagaggaaggcagctgggtttaggaagtgatggcaaggaaggctaatattggcttaagaggttggatcgcatctgatttctcacacagagaaaagggactaggttcaggacccagggtcagaacctgggtcattcaccttccaccttggtgacttggtcaagtttacagaacccctctgactcagctttctactctgtaaaatggggataatcataatcccctggccatgctggtggagtttagatctataagctcacacatgtgaaatgtcaggtgtagcatgtgcatcacaagggtatttctagatatccttcatgactgtggcttacatcatttcttcaaacatttggagaagaggacgagatggataaggttgtcatcccagcttttcactcgagaaggtggtggctcagtggggttaaatggcttccttgcactcctggaggcaggaagtggcaggtagggcagcggagaagtttagagtctcgtgtgtgtgtgtgtgtgtgtgtgtgtgtgtgtgtgtgtgtgtgtgtgtgtgtgtatgtgtgtgtgtgagtgtatcaggagagaggggtcggtgtaatggaggagactcaggtctgccatggaccttgtggctgttctcagagtgacttgacccaggacaaacgccccactgagtgtctttcaggtagtgactccatgatcacaatgctggttccatgctagggctggagccaggtaccttcctaacaactgcacatcttattttagggttgggtgtgtcttgctcttcctcatcctttgggtgctggaacagcatagaatatgtttcaagagttgctcactcaaagatttcatggactgggagcgtggctgagtgtagaatgcttgcctggtattcacagggctctggattcaattctagtgctgcttaagagggaggagggaggctcatgggaacaggcatgtcatgagtactgcaggtcagtggagggggtctagtgtccttacatacctatgtttgtcaacagctttcaaataatacatggtgggccaggtgtggtggcacaagtgaatctctgtggtttcaaggcctacagagagagttccaggacagctagggctacacagagaaaccctggttttttgtttgcttgttttgggggatttttttgtattttgttgttgtttttaaagacactgggtaggggctcagctgacttgccacaggacattgctcgcccagtgtaattgccaaaccttccgattttacaaaaaaagccctaacctggaaacacatatgaatctccctgttacatgtttgcctaaatattaaaacaaaatctccttaaaaaaaaaaaaacttcactttcacacaggtttgaatgagccacattttggcgaccaattctatttttaaggactggcaaggtcctttccctcctcagcatcggattagcagaacaaagcgtagcagctgtcagtcttgggatgtggcagaagaatactcttcctcctttgcactctgtgttctttaaaaaattgtattttattaatttattcattcattcgtgtatgtgcatgtcccaagcacatatgtgggggtcagaggacaacttgtggcagtcagttctcttgttttgctttgtggttcccaggggtcatttgggttgtcagtcttggtggcaagtgcctttacccctgagctccaattgattacaatatctggactctagagtcttgagccactggccaccctagaaacttgattttcacccattcctcatttactgtcccctttctatgcagaaaaagaaaaaaaacaaggtgagtgggccaggcactgtgcttatgtgtgcacatgcatgcatgtgtgtgtacacatatgtaacatgtggcacgtgtatgtatacttgtgtatatgcatgcatatgcatacacatgtgtaacatgtggcacgtgtgtgtatactcatgcatatgtatacacacacgtaacatgtggcatgtgtgtatacttgtgtatatgcatgcatatgtatacacatgtgtaacatgtggcatgtgtgtatatactcatgcatatgtatacatgcgtgtaacatgtggcatgtgtgtatacttgtgtatatgcatgcatatgtatacacatgtgtaacatatggcatgtgtgtgtacactgtggcatgtgtaagatgcacttcttcctcttttccttggagcctctgctgagttgggtgccctcgcaagtgcttcagctcagcctcacagtgatcctggactgtggttccattatcagtcctgtgtttagagggggaaaaggcagagacaaaccagtctccctgagaccacacttagccagggggttccacagagtctgtgctctgaaatacacagccgtcagacaaggtttggtgtgggtgcttatggtggtcctgaggaatctatctcagaggttctcctggcacgagtgctggtccagcccagagggaacatataccatgtgaagtgcttatagcaagcccctaccccactatataatagtatctataaggtagtcactgtttatgctttttataagtgagcgttcacattcagagaagcaaagtgatttgccacacagttagcaactggggacattggaaccacagtcccacatatgtctactccgagaccagtctcctgcctcacctccccagtgctccctacccttcaaggcttgtgttgtcaagattgcatgtgagtctataagcgcatggctggtagagatgccccatggctacaccctgcccactagtctgctcagaccgtcaccttcgcttgttctgagatcccaactagtttcatttgctttggcaagagccgttgtttataaaagcaagctgcggtctaaggagcctctcagagctaaacccgactctctctggtgctccccctgggtgagctgcacaggaacagagtggggcctccctgaccaggctgctttttttagtggaggctgctaagattatctgcactttggcccaagccctgctgtttcccaccacaccagcacaggcaggatgtggtggccactctctagctcttctgtggcctctgcacttgcatgaatatggccttaaggtagccctcctgcttctgcagtaccttcggggtttgcacagtaagtacagtttcactaaccctctggcttccgcagaggccctatcagaagggaaagtgaggttttaccaggtgcagtagcagatgagtgtgaacagccactgaaggtagttcttacatgaggggaggagggctgggcagagcagacatgccacaagcagcaggatatgagaggttcctggggaactgcagggccagagctctgttttctactaggatgaccttgaatttgtggtcattccctctgctagggttacagttgtgtgccaccacgaccagcctgaaagcctcatttttaatgggtttgacactgaatttctgggagacttcagtgtttttccacttgaaaaataaattcacaaaagtacttgataatcaccgatcacatcatctgtgctcagcattcttagcgcttttgcgtgttacttaggaacgcacaaggagaaatgggaggtgggatacaatagcggaccatccaacttatagaacatattagctgtttttaaaaatatgaataaaactgagaagaactgaggttcaagccacacatgatggtatacacaggggctagactgagctatagtgggattcagtcccaaacaaaacaaaacacagagagggagggagggagggacagacagacagacagacagagacagaaatggagagagacagacagagacagagagagtactcttcaaggtccagaaaggtttggcatgtctcctagccacacggcttgtgagttcagaaatgggactcaggcttctgagtgaccatttcctcctgagtggacaaagctgcttcaggggtcgtctgcctcttgtgtccgtcggcctgtaatacttgcttccctcctgcccgcccctttccctcccctcccctccccagcccatcatcttcgtttctgacagagcaaacagcaacaaggagctgggtgtggaccaggaggcagaggagggcaaagacaaagcagggcccgagaaacgaggggaaaccccacaggtttctgagcatgcggcatggatggggagtttagctcgtagtggcggctctgctatgctcttggggtccttaggggttatggtagctctcaggggagggtctcttcagcactgtggggtttggaattgtctgctgacttttcctcccggcaagttcctctgaactttgcaggtatttcttgggtaagtgtcttttaaggtcgctgccacaccaaaccagtcccagggtgggaaggataggccaagaacagcccactgacgcctctccagtcaggacaatggagcatggggagacaaaggctgagtgaggcgagccagctgcagagaacgaagctgaactgtctttgagttgttgcaaagggaggaaaaggggatggtagcagaaagtggggtctgacagcagccctgaagtcctgcagaactccagggccacgccctccatcccagagaacctaccaggtggggagggaggggtcctttgaggacttagctacagagacagtagcggttggaggacctggcctctggtcaggctcaaaagagcagagaaagttctatctggaagagagtcgggagagtcaaggatgctgtctgcatgttagcatggcaaatttgtcctttggcctgaggttccgggactgcagagcctctagaatcctcactgagtttgcaggtagactgaaaacttgaaccttgtgccacctgctgctgctttatggggctgtttgggatactgacgcctgggttcccataaaccagtttcctttgtgtccctatagccaaggcctggccacacagaccaggaaagtgaggagcagcagcaattccggaacatcttcaggcagatcgcaggcgacgtatgtatctctatcctagcccagggacccacctcctcaacgactcatcgtctaaaaggggtcatttgatgtgggagaaccagagaatgtgtgatgcatcaaagaaacctgtgcctttgggttcaaccactgactgggtggctcagagccttctggctgtgagctgcctgctgcactggcctttacaattctcttctctccctcaggacatggagatctgtgcagatgaactcaagaatgtcctcaacacagtggtgaacaaacgtgagcgacccacgccggatgtgggggatgggcaggggccccagctgtctgaaagcagctcctcacccttctccatccccccagacaaggacctgaagacacaagggttcacgctggagtcctgcagaagcatgatagctctcatggatgtatccttccatctgcccagagggtcaggggaacagttaagggtgtccacgcccagggaaagacttaagggaatcctaacacagatactcctggatatctggcagaggttaggaatggatactgggtagtggctggtcatcacctctggctcttaccacagatttgaaaccctgcttggcatgctgcccaaacttcctagcgggcaggctctgtgagctgtaaagctggcaggaactctggttaagggaggatagaacctggcagagtcaggggccctggtaaaggtggtttaaccaccttccctgcatagtgccctagacacccccaccccgttcagaggcattttactatgtgcctgtggtccttacatctatcctctagacagatggctctggaagactgaatcttcaagagttccatcacctctggaaaaagatcaaggcctggcaggtgcgatgagagacggatgtgggaatgatggacagggttaatttgggattaagtgtgtgattgctgtctttgaactttgtattttcagaaaatcttcaagcactatgacacagaccattccggtaccatcaatagctatgagatgcgaaatgcagtcaatgatgcaggtgctggggtgcaggggatgctggggggttgagagggtgacaacatgcagacctgagagctgggggaagcaggaggggatgggaaaggagactgcaagcctgagaacttcatccctccctctccctgcccctccaaggcttccatctcaacagccaactctatgacatcatcaccatgcgctatgcggacaaacacatgaacatcgactttgacagcttcatctgctgcttcgtcaggctggaagggatgttcagtaagtgaggggctactccccagatttctcaagcccccccccccaggctccttcctcatagtacagccacaacaagagctcagcctgacagttgcccactcttccactgaggctgcatgtcagccgttactctctgaaggccaagaaaaggatggcaaagggagacgaaagatgcaagggagagatagatgctggttacgtcctgcatggatctgtgtggaaggatagcactcagcacagaggcttcttaaattacagatctgagcactcagatgatgctcacgtgtgatcccagcagttaggtaggagagataggaggattatgaattcaaggccagcaggggctacttggagagaccttgtttcaataataaaacaagggctggcaagatggctcagctgcatggctctggtggtctgagtttaatggctgaaatccatgaggtagaaggagaaaaccaatctcagagttgcccctctgacctctacatatgttctgtgcccacatgcatacagtaataaattcttcaagcagcaaacacaagtaaactaggtccaaaccctaagaaaaggaatgggaggtgacaaatgagagtctgtgactagcatttgctttatgctttcacacaggagcttttaacgcatttgacaaggatggcgatggtatcatcaaactgaatgtacttgaggtaagtgcagcaaacacatcctctgcacagaacccacagggggtgggagggatgaatgacaggcagaacccagcacgcttctcactctcaggactggaacttggtgaagattgcgattcctggggtgggtgtacaacagaggctagccccacctctgagttgcacctcggtggaagatactttcctagggaagacccagggaaggcaggctgctcctttaaggctcctccctttctccgtcccagcacaagcaattcctagggatggctgttacctccttgtttgctcttgtactgatctcagggtttcttcttccagtggctgcagcttaccatgtatgcctgaaccagactgacctcatgttaagagcaactaggatttcatctcaacacaacacagctagagccatctaccacaaaagacccagtaggtgcatctccaccaaacctagggggctcctggtcatgttccttctccattttgcaccaactcttggtgcacagcctgatgccacctgaagtgtctggcttgctgagaggtctgtagattctgaaactgtctacttagcttgtactgggttgtccatagcacctgtagatggctctcactcacggaggagcccagtcgccatctgcactcagttctgccacttttagggtgggtctattctggggtcaatagggcgtggatatctgacaaaaatgtggctacattctgaaagaagctatctaaataaaggcacacacatggctagttccaccattctccttattagcatttctcagccacaggaggactgagtatcttccagtcatatttcatgcatcacctccacaaagatctcgtttggggatagggagatagttgatacaggtctacgcaaccacgaggagcccagtggtagcttacacctgtaactacagtaatggagaacaatgacaaggggaattctgcaggcccactggccagtcaagccaaaaggatgaggtttggtacaagagagcctggatacggtggagggactgaggagcaatggcttacggggtaaaattgctgtgtttggatccccagggcccacacaaatgtaaatacttgttggagtaatcacccacctgtacttccatcccaagggggccagagaaagattactgacaaaaagccaattagctagactagctgacctggcaagctctgggttcaatggagagacctggcctcagtgtggaaggcagaaggtaacatcaacctgacctccacaagcatgcacacaaacataaaggaaatatccaagtctatctgacctctgctcctgcgcacatcacaaagaaaacccttttggcctatgcacatacctggtcctgtcaggtattgaaagggttcggtctggttagtgttggcatatttcttctttaatttttttgggtttctatatctctccctcccatccaactcttattccaccctcgttccttccaatccaccaacattagataggagataaaggtaagaggggaaagagggcatagacctctttagactcgttcctgctgattaggggtgttgggttccttggggcaagtccaatcttcatcaacagtatatttcctgtctctttgtccatgacttgacaaaccccaagaaccaggaacagcaaggggaagctgcagcttccatgggccctttcaggtctgttccacttataccctctccagaattaaactatctgcagctggcaaagccacacgcttgctagtgcatg

SEQ ID NO: 3 - DNA, 20 bp, artificial sequence
  siRNA 1
tggaagaagacctccggaaa

SEQ ID NO: 4 - DNA, 19 bp, artificial sequence
  siRNA 2
cccatgatcaaagtttcat

SEQ ID NO: 5 - DNA, 23 bp, artificial sequence
  siRNA 3
aacctctccttctggtctgaaca

SEQ ID NO: 6 - DNA, 19 bp, artificial sequence
  siRNA 4
ccaaagagatgcacgggaa

SEQ ID NO: 7 - DNA, 19 bp, artificial sequence
  siRNA 5
gcaacaaggagctgggtgt

SEQ ID NO: 8 - DNA, 19 bp, artificial sequence
  siRNA 6
acaaggacctgaagacaca

SEQ ID NO: 9 - protein, 4069 aa, human
MASRDSNHAGESFLGSDGDEEATRELETEEESEGEEDETAAESEEEPDSRLSDQDEEGKIKQEYIISDPSFSMVTVQREDSGITWETNSSRSSTPWASEESQTSGVCSREGSTVNSPPGNVSFIVDEVKKVRKRTHKSKHGSPSLRRKGNRKRNSFESQDVPTNKKGSPLTSASQVLTTEKEKSYTGIYDKARKKKTTSNTPPITGAIYKEHKPLVLRPVYIGTVQYKIKMFNSVKEELIPLQFYGTLPKGYVIKEIHYRKGKDASISLEPDLDNSGSNTVSKTRKLVAQSIEDKVKEVFPPWRGALSKGSESLTLMFSHEDQKKIYADSPLNATSALEHTVPSYSSSGRAEQGIQLRHSQSVPQQPEDEAKPHEVEPPSVTPDTPATMFLRTTKEECELASPGTAASENDSSVSPSFANEVKKEDVYSAHHSISLEAASPGLAASTQDGLDPDQEQPDLTSIERAEPVSAKLTPTHPSVKGEKEENMLEPSISLSEPLMLEEPEKEEIETSLPIAITPEPEDSNLVEEEIVELDYPESPLVSEKPFPPHMSPEVEHKEEELILPLLAASSPEHVALSEEEREEIASVSTGSAFVSEYSVPQDLNHELQEQEGEPVPPSNVEAIAEHAVLSEEENEEFEAYSPAAAPTSESSLSPSTTEKTSENQSPLFSTVTPEYMVLSGDEASESGCYTPDSTSASEYSVPSLATKESLKKTIDRKSPLILKGVSEYMIPSEEKEDTGSFTPAVAPASEPSLSPSTTEKTSECQSPLPSTATSEHVVPSEGEDLGSERFTPDSKLISKYAAPLNATQESQKKIINEASQFKPKGISEHTVLSVDGKEVIGPSSPDLVVASEHSFPPHTTEMTSECQAPPLSATPSEYVVLSDEEAVELERYTPSSTSASEFSVPPYATPEAQEEEIVHRSLNLKGASSPMNLSEEDQEDIGPFSPDSAFVSEFSFPPYATQEAEKREFECDSPICLTSPSEHTILSDEDTEEAELFSPDSASQVSIPPFRISETEKNELEPDSLLTAVSASGYSCFSEADEEDIGSTAATPVSEQFSSSQKQKAETFPLMSPLEDLSLPPSTDKSEKAEIKPEIPTTSTSVSEYLILAQKQKTQAYLEPESEDLIPSHLTSEVEKGEREASSSVAAIPAALPAQSSIVKEETKPASPHSVLPDSVPAIKKEQEPTAALTLKAADEQMALSKVRKEEIVPDSQEATAHVSQDQKMEPQPPNVPESEMKYSVLPDMVDEPKKGVKPKLVLNVTSELEQRKLSKNEPEVIKPYSPLKETSLSGPEALSAVKMEMKHDSKITTTPIVLHSASSGVEKQVEHGPPALAFSALSEEIKKEIEPSSSTTTASVTKLDSNLTRAVKEEIPTDSSLITPVDRPVLTKVGKGELGSGLPPLVTSADEHSVLAEEDKVAIKGASPIETSSKHLAWSEAEKEIKFDSLPSVSSIAEHSVLSEVEAKEVKAGLPVIKTSSSQHSDKSEEARVEDKQDLLFSTVCDSERLVSSQKKSLMSTSEVLEPEHELPLSLWGEIKKKETELPSSQNVSPASKHIIPKGKDEETASSSPELENLASGLAPTLLLLSDDKNKPAVEVSSTAQGDFPSEKQDVALAELSLEPEKKDKPHQPLELPNAGSEFSSDLGRQSGSIGTKQAKSPITETEDSVLEKGPAELRSREGKEENRELCASSTMPAISELSSLLREESQNEEIKPFSPKIISLESKEPPASVAEGGNPEEFQPFTFSLKGLSEEVSHPADFKKGGNQEIGPLPPTGNLKAQVMGDILDKLSEETGHPNSSQVLQSITEPSKIAPSDLLVEQKKTEKALHSDQTVKLPDVSTSSEDKQDLGIKQFSLMRENLPLEQSKSFMTTKPADVKETKMEEFFISPKDENWMLGKPENVASQHEQRIAGSVQLDSSSSNELRPGQLKAAVSSKDHTCEVRKQVLPHSAEESHLSSQEAVSALDTSSGNTETLSSKSYSSEEVKLAEEPKSLVLAGNVERNIAEGKEIHSLMESESLLLEKANTELSWPSKEDSQEKIKLPPERFFQKPVSGLSVEQVKSETISSSVKTAHFPAEGVEPALGNEKEAHRSTPPFPEEKPLEESKMVQSKVIDDADEGKKPSPEVKIPTQRKPISSIHAREPQSPESPEVTQNPPTQPKVAKPDLPEEKGKKGISSFKSWMSSLFFGSSTPDNKVAEQEDLETQPSPSVEKAVTVIDPEGTIPTNFNVAEKPADHSLSEVKLKTADEPRGTLVKSGDGQNVKEKSMILSNVEDLQQPKFISEVSREDYGKKEISGDSEEMNINSVVTSADGENLEIQSYSLIGEKLVMEEAKTIVPPHVTDSKRVQKPAIAPPSKWNISIFKEEPRSDQKQKSLLSFDVVDKVPQQPKSASSNFASKNITKESEKPESIILPVEESKGSLIDFSEDRLKKEMQNPTSLKISEEETKLRSVSPTEKKDNLENRSYTLAEKKVLAEKQNSVAPLELRDSNEIGKTQITLGSRSTELKESKADAMPQHFYQNEDYNERPKIIVGSEKEKGEEKENQVYVLSEGKKQQEHQPYSVNVAESMSRESDISLGHSLGETQSFSLVKATSVTEKSEAMLAEAHPEIREAKAVGTQPHPLEESKVLVEKTKTFLPVALSCRDEIENHSLSQEGNLVLEKSSRDMPDHSEEKEQFRESELSKGGSVDITKETVKQGFQEKAVGTQPRPLEESKVLVEKTKTFLPVVLSCHDEIENHSLSQEGNLVLEKSSRDMPDHSEEKEQFKESELWKGGSVDITKESMKEGFPSKESERTLARPFDETKSSETPPYLLSPVKPQTLASGASPEINAVKKKEMPRSELTPERHTVHTIQTSKDDTSDVPKQSVLVSKHHLEAAEDTRVKEPLSSAKSNYAQFISNTSASNADKMVSNKEMPKEPEDTYAKGEDFTVTSKPAGLSEDQKTAFSIISEGCEILNIHAPAFISSIDQEESEQMQDKLEYLEEKASFKTIPLPDDSETVACHKTLKSRLEDEKVTPLKENKQKETHKTKEEISTDSETDLSFIQPTIPSEEDYFEKYTLIDYNISPDPEKQKAPQKLNVEEKLSKEVTEETISFPVSSVESALEHEYDLVKLDESFYGPEKGHNILSHPETQSQNSADRNVSKDTKRDVDSKSPGMPLFEAEEGVLSRTQIFPTTIKVIDPEFLEEPPALAFLYKDLYEEAVGEKKKEEETASEGDSVNSEASFPSRNSDTDDGTGIYFEKYILKDDILHDTSLTQKDQGQGLEEKRVGKDDSYQPIAAEGEIWGKFGTICREKSLEEQKGVYGEGESVDHVETVGNVAMQKKAPITEDVRVATQKISYAVPFEDTHHVLERADEAGSHGNEVGNASPEVNLNVPVQVSFPEEEFASGATHVQETSLEEPKILVPPEPSEERLRNSPVQDEYEFTESLHNEVVPQDILSEELSSESTPEDVLSQGKESFEHISENEFASEAEQSTPAEQKELGSERKEEDQLSSEVVTEKAQKELKKSQIDTYCYTCKCPISATDKVFGTHKDHEVSTLDTAISAVKVQLAEFLENLQEKSLRIEAFVSEIESFFNTIEENCSKNEKRLEEQNEEMMKKVLAQYDEKAQSFEEVKKKKMEFLHEQMVHFLQSMDTAKDTLETIVREAEELDEAVFLTSFEEINERLLSAMESTASLEKMPAAFSLFEHYDDSSARSDQMLKQVAVPQPPRLEPQEPNSATSTTIAVYWSMNKEDVIDSFQVYCMEEPQDDQEVNELVEEYRLTVKESYCIFEDLEPDRCYQVWVMAVNFTGCSLPSERAIFRTAPSTPVIRAEDCTVCWNTATIRWRPTTPEATETYTLEYCRQHSPEGEGLRSFSGIKGLQLKVNLQPNDNYFFYVRAINAFGTSEQSEAALISTRGTRFLLLRETAHPALHISSSGTVISFGERRRLTEIPSVLGEELPSCGQHYWETTVTDCPAYRLGICSSSAVQAGALGQGETSWYMHCSEPQRYTFFYSGIVSDVHVTERPARVGILLDYNNQRLIFINAESEQLLFIIRHRFNEGVHPAFALEKPGKCTLHLGIEPPDSVRHK

SEQ ID NO: 10 - protein, 3739 aa, murine
MESGDSGLAAQGFLGWGADEEVAQELETEEESEGEGEETAAESEEEPDARLSDEDEEGKTKQECIVSDPSFSMVAVQREDSGITWETNSSRSSTPWASGESQTSGICSLEGSALTSPPGSVSFIMDEVKRTRKRTQKSKRGSPSLRRKGSKKRNSLESQDVLTNQEDGPSISESPVLNIENEKSSIGTYDKTRRKKTASNTPPITGAIYKEHKPLVLKPVYIGTVQYKIKMFNSVKEELIPLQFYGTLPKGYVIKEIHYRRGKDSSISLEPDLSNGGSNIVPQRKLAQSPEEDKVRELAPPWRGALSKGSRTSLFSHEEQKKTYADSNLNVPSSTEHAFPSSARNDTADQEENLSLPQMMPQQPADESKTHRMEPPSIPATMVLERAKEELEQNAQGKESSEDDASVLTGSADDVQQEGLVSVNHSMPWEAEKESLETGPPRPAPAIQEKFEPDMEGLEPISTEKTEQASEYVTSSEPIVHREEEHAPEPIVHREEEHAPEPIVHREEEHAPEPESIVHREEEHAPESIVHREEEHAPEPVPIVHREEEHAPEPESIVHREEEHAPEPIVHRDKGHALEPIVHREEEHAPEPIVHRDEGHAPEPIVHREEEHVPEPESIVRKGEEHAPEPIVHREEEQVPEPESIVHREEEHAPEPIVHREEEQVPEPESIVHREEEHAPEPMVLREEHAPEPIVRREEEHAPEPIVHREEEHAPEPMVHRKAQQLERGVETSTPITDITEPEDSSLEEEIIELDYPESPLASKETSPSPLSPEVEHRKEPILPTQMTFTPERITLSEEEREENESVSTDSAFVSEYSVLQDLNHTPEKLEVEAVSVSDVKSSNEPAVFSEDDEERESYSPAMTSVSEQSLSPSTTEKTSAIQSPLFSTVSPVLSGDEASENVCHSPESESAAEYSVPAHAQELLLKTGDHKLPLKSQRVSEPIIQAEDEKEDIGLLPPAALSQAVLSEDESLGSGSFASDSKLPFKPSVSQNATRESPQKTIDDMPQFKPRGLSDPATLLEEEKEAIGVGLSSSNEVSAVECALPPQTTELLSESHAPPPWAISSEQVVQSEEGSRDQQRGSFSSTPELGHTSLLLKGASSPTGLSEQGQEEDNIGPLSPDSAFASEFSFSPYPTQELEKRELGRDSPLCLTSPSEQTVLSEEDTEEADLFSPDSASQVSIPPYRIAETEQNKVEPDELLPTRSAPDYPYFSEADEEEAGSSVVTLVPEHSEPSQEREESSPCRPVFEDLSLPPSADKTGQAETMSDVPTISTSVSEYLILARQAKTQASLEPEAEDLVPPPTSGWEKRDAKSSLPAVTIAASSSALSSVVKEETTSVLPTSQPSVSPESTCVLKPEQEPTAPLTLTSADEQMALPRVGREKAVLDSQEATAHKSQDQTPEPRLPNVPGSGMKYSVLSDLGDEPKADVKLNLAPTVTSELEQRMLSKNEPEVAKPHSPPEETSISGPKVLSAVKTEVKQESKITRELPAASSGRERGAEHSPPVPPALPALTEETGKDTEASSSATTVPVTKLDSNSTKLGRDEVLTDPSLASPVEHPGLKGIGKSELGSGLPLPSMSASEVLRPEPKLPVNSGVEVEREDNEPPPLQVSPTSKPTVPNDKHEEITRSPDSENLVSDDLAPTLLAFRHEMNRQAEETSSPVPGSFLSGEQELIKLPPEPEKHKQLSEVPTAGSELIDSRDRDRSLGIEPVKPIGTEPGPSILEKGPAELQRRGKEQEENRKLPVPASAPLETASFDLPIEQKEPKRTLHEGQAVEVPDESSSSADKPELGVKQLAEKKENLEQPKPFVTTERASVTGSKVKESLISPKDNIWMLEKPDGLVNQHEDRKPGTGQLESSESTDLMSEKLGAASLDTDHTSETRNQETSKAPVSGEKLSQEPRRVQSKAVDDSEEGRKLASGNVEVLTQSKSVPAVKAKATPQPPETPEVTQKPSEKSLVTEQGLPAEKGKKGISSFKSWMSSLLFGSSIPDSKVSDNEDLETRPGPSVEKAVPAIEPKGTVPAEVNIAEKPAVHSLPEVTVKLAEEPKGVSVKSSISQDLKEKLTFLSNEDVLKQPKSNSENYGQKELPGFSEGMGESLATSVGDKHPGIHPCSPMGEKVGMEEAQNMAPLHITESQRRQKPEVSPPSMWNISARKEEPSSDHKETWLSSSDVVDRMPQKPKSAQSAFTRMNSEEPASMILPVESKGSLSDLGEDRLRQEMPKPTSLEHCEEEVERPTEEKDGWETRSFSLAGKRGLAEKQEIMAPLELRENEAVGELQRMPESRPFKLEESKAAERLEQRISPTEKLMEKPSKTLALDRREKEVQEWVFSEGEKQEYPPAAMPVPGASAVSLDKAQPHLLAKPTPVVEKPEHIVTEVYPEIRERKAAETQPHPQEEGKTLVEKTKVSRVESPHGEETDGHSLTQEGNLELEKSGESRVDLKEERRRFVMPELPLGASVAAEDGSVQPRPLSKDAARASDMTDETKHLGTPPTQPSAVEPQTLVLGTSVEHAVKKQETWSDRPTVHTFQTSKDDTEEMLKQSVLISKHHLEAVEDVHRNEPPSSAASNYAQFMLSASEISADGVPPMGGTAQEPEGTSVKDEEFSVTSKPAGLSEDQKSAFSIISEGCEILNIHAPAFIPSVDQEESEQMQDKLQYLEEKASFKSISVHDEKKAAASHKTQKSKLEVPDRKITSLKENKTKETHKTKEEIATDSGMGDFTPIQPTVSGEEDYFEKYTLIDYNLSPGSGKQKSTVEESSEEATKTLTSFPESSAEQALDHEYNLVKLDESFYGPEKDDSKLSHAEMQKSLAIQKPDDRNAPKGISRDVDSRSPGMPLFDVEEGVLSKRQIFPTTPKAVNPELLEEPPALSFFYKDLYEGACGEKNEGETASEGDSVDSETSFPRRHSDTDDGPGMYFEKYILKDDILHDESVTQEDQGQGLEEKPVGEEDSQQLRVAEREIRRKPETSFWEKNLEEQHKVVGREGEPTGHMETLDEAAMQQKAPITEQVRAVTQKMSYAVPFQDTRCVLESEPSSQGNEAGNASPDVNLNVPVQVSFPEEESAAGATYAPEVLQERLVPSVSREERLHNTPVQDEYDFVGSLNQEAASQAILPEEPGSESSPKEVLSQGSESFEHIREQELTSEGEPRMSASQEVWDRTEDQSARESVTAKTQKEPKKTQAESYCYTCKSLVSEMDKALDIHKDHEVSALDTAISAVKVQLGEFLENLQEKSLRIEAFVSEIESFFNTIEEKCSKNEKRLEMQNEEMMKRVLAQYDEKAQSFEEVKKKKMEFLHDQMVHFLQSMDTAKDTLETIVREAEELDETVFLASFEEINERLLSAMESTASLENMPAAFSLFEHYDDSSARSDQMLKQVAVPQPPRLEPQEPSSATSTTIAVYWSVNKEDVVDSFQVYCVEEPQDDQEINELVEEYRLTVKESCCIFEDLEPDRCYQVWVMAVNFTGCSLPSERAIFRTAPSTPVIHVEDCTVCWNTATVRWRPANPEATETYTLEYCRQHSPEGEGLRSFSGIKGHQLKVNLPPNDNYFFYVRATNASGTSEQSEAALISTRGTRFLLLRETAHPALQISANGTVISFSERRRLTEIPSVLGEELPACGQHYWETTVADSPAYRLGICTSSAVRAGALGQGETSWYMHCSEPQRYTFFYSGIVSEVHATERPARVGILLDYTNQRLLFINAESGQLLFIVRHRFNEGVHPAFALEKPGRCTLHLGLEPPDSVRHK

SEQ ID NO: 11 - protein, 317 aa, artificial sequence
  human MD9
YFEKYTLIDYNISPDPEKQKAPQKLNVEEKLSKEVTEETISFPVSSVESALEHEYDLVKLDESFYGPEKGHNILSHPETQSQNSADRNVSKDTKRDVDSKSPGMPLFEAEEGVLSRTQIFPTTIKVIDPEFLEEPPALAFLYKDLYEEAVGEKKKEEETASEGDSVNSEASFPSRNSDTDDGTGIYFEKYILKDDILHDTSLTQKDQGQGLEEKRVGKDDSYQPIAAEGEIWGKFGTICREKSLEEQKGVYGEGESVDHVETVGNVAMQKKAPITEDVRVATQKISYAVPFEDTHHVLERADEAGSHGNEVGNASPE

SEQ ID NO: 12 - protein, 314 aa, artificial sequence
  murine MD9
YFEKYTLIDYNLSPGSGKQKSTVEESSEEATKTLTSFPESSAEQALDHEYNLVKLDESFYGPEKDDSKLSHAEMQKSLAIQKPDDRNAPKGISRDVDSRSPGMPLFDVEEGVLSKRQIFPTTPKAVNPELLEEPPALSFFYKDLYEGACGEKNEGETASEGDSVDSETSFPRRHSDTDDGPGMYFEKYILKDDILHDESVTQEDQGQGLEEKPVGEEDSQQLRVAEREIRRKPETSFWEKNLEEQHKVVGREGEPTGHMETLDEAAMQQKAPITEQVRAVTQKMSYAVPFQDTRCVLESEPSSQGNEAGNASPD

SEQ ID NO: 13 - DNA, 29 bp, artificial sequence
  amorce TTN 1180
gctatctgcacctcaaaatctgtgggttg

SEQ ID NO: 14 - DNA, 26 bp, artificial sequence
  amorce TTN 1183
gaaccctgaccctctggaagaacatc

SEQ ID NO: 15 - DNA, 24 bp, artificial sequence
  amorce GW255
agtcttccttccaaagttgcctgc

SEQ ID NO: 16 - DNA, 24 bp, artificial sequence
  amorce GW257
gtgctacttccatttgtcacgtcc

SEQ ID NO: 17 - DNA, 24 bp, artificial sequence
  amorce GW259
acttctctgaagcaaactccagcc

SEQ ID NO: 18 - DNA, 21 bp, artificial sequence
  ex3_BP
acagaucgugagcacuccugc

SEQ ID NO: 19 - DNA, 22 bp, artificial sequence
  ex3_ESE1
ggcugcgagaaaccagcagucc

SEQ ID NO: 20 - DNA, 22 bp, artificial sequence
  ex3_ESE2
ucccugcguaguuuucgaugaa

SEQ ID NO: 21 - DNA, 21 bp, artificial sequence
  ex4_BP
auuacuguuaggaaauguguc

SEQ ID NO: 22 - DNA, 23 bp, artificial sequence
  ex4_ESE1
acguuggcaggcagucaucuaua

SEQ ID NO: 23 - DNA, 22 bp, artificial sequence
  ex4_ESE2
uccagaacucauugcggugguu

SEQ ID NO: 24 - DNA, 21 bp, artificial sequence
  ex5_BP
agaccaauuuggggucacaga

SEQ ID NO: 25 - DNA, 23 bp, artificial sequence
  ex5_ESE1
cugugaaguccuccauggccucu

SEQ ID NO: 26 - DNA, 22 bp, artificial sequence
  ex5_ESE2
uggcuuucuucaugaucuugua